(19) United States
(12) Patent Application Publication
(10) Pub. No.: US 2020/0199568 A1
(43) Pub. Date: Jun. 25, 2020

(54) METHODS FOR EXPERIMENTAL EVOLUTION OF NATURAL AND SYNTHETIC MICROBES

(71) Applicants: Massachusetts Institute of Technology, Cambridge, MA (US); Trustees of Boston University, Boston, MA (US)

(72) Inventors: Caleb J. Bashor, Boston, MA (US); Jason Hung-Ying Yang, Watertown, MA (US); Arnaud Gutierrez, Somerville, MA (US); Wooseok Steven Ahn, Tenafly, NJ (US); James J. Collins, Newton, NJ (US); Brandon Gei-Chin Wong, Brighton, MA (US); Ahmad S. Khalil, Lexington, MA (US)

(73) Assignees: Massachusetts Institute of Technology, Cambridge, MA (US); Trustees of Boston University, Boston, MA (US)

(21) Appl. No.: 16/081,975

(22) PCT Filed: Feb. 17, 2018
(86) PCT No.: PCT/US2018/018547

§ 371 Date: Sep. 4, 2018

Related U.S. Application Data
(60) Provisional application No. 62/460,121, filed on Feb. 17, 2017.

(51) Int. Cl. C12N 15/01; C12N 1/36; C12M 1/34; C12M 1/36; C12Q 1/02

(52) U.S. Cl. CPC C12N 15/01; C12N 1/36; C12Q 1/02; C12M 41/48; C12M 41/36; C12M 41/46

(57) ABSTRACT

Disclosed herein are a high-throughput continuous culture system and novel methodologies for the experimental evolution of natural and synthetic microbes using the continuous culture system. The microbial culture is exposed to a stress ramp function which is overlaid on top of a culture fitness function. The amount of stress applied to the culture is increased in response to increased fitness of the microbial culture.

### RELATED APPLICATIONS

This application is a national stage filing under 35 U.S.C. 371 of International Patent Application Serial No. PCT/US2018/018547, filed Feb. 17, 2018, which claims priority under 35 U.S.C § 119(e) to U.S. provisional application No. 62/460,121, filed Feb. 17, 2017, the contents of each of which is incorporated herein by reference in its entirety.

### GOVERNMENT SUPPORT

This invention was made with Government support under Grant Nos. HDTRA1-15-1-0051 and HR0011-15-C-0091 awarded by the Defense Threat Reduction Agency, and Grant No. MCB-1350949 awarded by the National Science Foundation. The Government has certain rights in the invention.

### FIELD

Disclosed herein are novel methodologies for the experimental evolution of natural and synthetic microbes using a custom, high-throughput continuous culture system, and such continuous culture systems.

### BACKGROUND

A number of “do-it-yourself” (DIY) continuous culture (CC) system designs have appeared in the literature in recent years (Toprak E., et. al., Nat. Genet. 44, 101-5 (2012); (Acar M., et. al., Nat. Genet. 40, 471-75 (2008); https://depts.washington.edu/soslab/turbidostat/pmwiki; http://openwetware.org/wiki/Turbidostat). A typical DIY CC system is comprised of integrated wetware, hardware, and software modules that can be readily interchanged and reconfigured. Maintenance of culture conditions (e.g., temperature, stirring speed, and media composition) can be automated over long periods (days to weeks). The modular customizability of DIY CC systems, and their ability to operate over long periods, enables the user to conduct a broad repertoire of evolution experiments. However, the configuration and application of DIY CC devices to areas of experimental evolution has remained under-explored.

### SUMMARY

Previously, most continuous culture (CC) evolution experiments were restricted to low throughput, limiting the ability to assess the relationship between culture evolvability and either culture conditions or parental strain genotype. Scalability of CC systems addresses this issue, as the effects of a variety of culture conditions on evolution can be tested simultaneously. Thus, CC systems can be used for a number of applications with potential commercial relevance, including testing new antibiotic treatment regimes for resistance acquisition or testing communities of strains for their ability to complement or support each other's growth.

As described herein, CC devices, in several different implementations, can serve as enabling technology for a number of under-explored areas in experimental evolution including selecting for novel functionality in a WT microorganism, improving engineered circuit stability by evolving both the circuit and host, testing and evolving stable multi-species communities, and testing engineered synthetic communities.

In some aspects, the disclosure relates to methods of performing experimental evolution on at least one fluidic microbial culture in a continuous culture system. In some embodiments, the method comprises: subjecting the at least one microbial culture to a dynamic environment, wherein the at least one microbial culture is exposed to a stress ramp function which is overlaid on top of a culture fitness function; and increasing the amount of stress applied to the at least one microbial culture in response to the increased fitness of the at least one microbial culture, wherein fitness is calculated in real-time.

In some embodiments, the fitness function ramp comprises more than one fitness measurement. In some embodiments, the fitness ramp function comprises a turbidity and/or a fluorescence measurement.

In some embodiments, the stress ramp function comprises more than one microbial stress. In some embodiments, the stress ramp function comprises an antibiotic, an antiseptic, a temperature, an aerobic, an anaerobic, an infectious, a nutrient, an irradiative, a pH, a metabolic, and/or a mechanical stress. In some embodiments, the stress ramp function comprises an increase or decrease in temperature.

In some embodiments, the at least one microbial culture evolves a novel functionality. In some embodiments, the novel functionality is selected from the list comprising stress tolerance, nutrient utilization, or metabolite production.

In some embodiments, at least one of the at least one fluidic microbial cultures comprises an archaea, a bacterium, a fungi, a protista, a microbial merger or symbiont, and/or a planarian. In some embodiments, at least one of the at least one fluidic microbial cultures comprises a suspension of mammalian cells, plant cells, or insect cells.

In some embodiments, the continuous culture system comprises integrated wetware, hardware, and software modules that can be readily interchanged and reconfigured. In some embodiments, the continuous culture system comprises a turbidostat with fluorescence detection. In some embodiments, the continuous culture system is configured to allow for vial-to-vial culture transfer. In some embodiments, the continuous culture system is configured to allow for continuous mixing of the microbial suspensions.

In some aspects, the invention relates to methods of testing the mutational stability of an engineered circuit. In some embodiments, the method comprises: subjecting a microbial cell comprising at least one engineered circuit to a dynamic environment, wherein the microbial cell is exposed to stress ramp function which is overlaid on top of a culture fitness function; increasing the amount of stress applied to the microbial cell in response to the increased fitness of the microbial cull, wherein fitness is calculated in real-time; and determining the time required for the engineered circuit to inactivate.

In some embodiments, the engineered circuit comprises a fluorescent output. In some embodiments, the fluorescent output is selected from the list consisting of TagBFP, mTagBFP2, Azurite, EBFP2, mKalama1, Sirius, Sapphire, T-Sapphire, ECFP, Cerulean, SCFP3A, mTurquoise, mTurquoise2, monomeric Midoriishi-Cyan, TagCFP, mTFP1, EGFP, Emerald, Superfolder GFP, Monomeric Azami Green, TagGFP2, mUKG, mWasabi, Clover, mNeonGreen, EYFP, Citrine, Venus, SYFP2, TagYFP, Monomeric Kusabira-Orange, mKOκ, mKO2, mOrange, mOrange2, mRaspberry, mCherry, mStrawberry, mTangerine, tdTomato, TagRFP, TagRFP-T, mApple, mRuby, mRuby2, mPlum, HcRed-Tandem, mKate2, mNeptune, NirFP, TagRFP657, IFP1.4, and iRFP.

In some embodiments, the fitness function ramp comprises more than one fitness measurement. In some embodiments, the fitness ramp function comprises a turbidity and/or fluorescence measurement.

In some embodiments, the stress ramp function comprises more than one microbial stress. In some embodiments, the stress ramp function comprises an antibiotic, an antiseptic, a temperature, an aerobic, an anaerobic, an infectious, a nutrient, an irradiative, a pH, a metabolic, and/or a mechanical stress. In some embodiments, the stress ramp function comprises an antibiotic. In some embodiments, the stress ramp function comprises an increase or decrease in temperature.

In some embodiments, the microbial cell is selected from the group consisting of an archaea, a bacterium, a fungi, a protista, a microbial merger or symbiont, and a planarian. In some embodiments, the microbial cell is selected from the group consisting of a mammalian cell, a plant cell, or an insect cell.

In some embodiments, the continuous culture system comprises integrated wetware, hardware, and software modules that can be readily interchanged and reconfigured. In some embodiments, the continuous culture system comprises a turbidostat with fluorescence detection. In some embodiments, the continuous culture system is configured to allow for vial-to-vial culture transfer. In some embodiments, the continuous culture system is configured to allow for continuous mixing of the microbial suspensions.

In some aspects, the invention relates to methods of testing the stability of at least one multi-species microbial community. In some embodiments, the method comprises: subjecting the at least one multi-species microbial community to a dynamic environment, wherein the at least one multi-species microbial community is exposed to a stress ramp function which is overlaid on top of a culture fitness function, increasing the amount of stress applied to the at least one multi-species microbial community in response to the increased fitness of the at least one multi-species microbial community, wherein fitness is calculated in real-time; and determining the fitness of each species independently.

In some embodiments, the fitness function ramp comprises more than one fitness measurement. In some embodiments, the fitness ramp function comprises a turbidity and/or a fluorescence measurement.

In some embodiments, the stress ramp function comprises more than one microbial stress. In some embodiments, the stress ramp function comprises an antibiotic, an antiseptic, a temperature, an aerobic, an anaerobic, an infectious, a nutrient, an irradiative, a pH, a metabolic, and/or a mechanical stress. In some embodiments, the stress ramp function comprises an antibiotic. In some embodiments, the stress ramp function comprises an increase or decrease in temperature.

In some embodiments, the multi-species microbial community comprises at least one of an archaea, a bacterium, a fungi, a protista, a microbial merger or symbiont, or a planarian. In some embodiments, the multi-species microbial community comprises at least one of a mammalian cell, a plant cell, or an insect cell.

In some embodiments, the continuous culture system comprises integrated wetware, hardware, and software modules that can be readily interchanged and reconfigured. In some embodiments, the continuous culture system comprises a turbidostat with fluorescence detection. In some embodiments, the continuous culture system is configured to allow for vial-to-vial culture transfer. In some embodiments, the continuous culture system is configured to allow for continuous mixing of the microbial suspensions.

In some aspects, the disclosure relates to methods of constructing a multi-species community comprising subjecting a multi-species community comprising microbial strains that comprise engineered circuits that facilitate cell-cell interactions to the method as described above.

In some aspects, the disclosure relates to continuous culture systems configured for high-throughput microbial evolution studies. In some embodiments, the continuous culture system configuration comprises at least one stress ramp function that is overlaid on top of at least one culture fitness function, wherein the relationship between the at least one stress ramp function and the at least one fitness function responds to increased culture fitness with increased application of stress in real-time.

In some embodiments, the continuous culture system comprises integrated wetware, hardware, and software modules that can be readily interchanged and reconfigured. In some embodiments, the continuous culture system is configured as a turbidostat with fluorescence detection to measure circuit output and track the loss or gain of circuit function over time. IN some embodiments the continuous culture system is configured to allow vial-to-vial culture transfer. In some embodiments, the continuous culture system is configured to allow continuous mixing of individual cultured species into a community culture. In some embodiments, the continuous culture system is configured for long-term continuous culture.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIG. 1. Overview of a generic DIY CC system. Modules comprising the system and their connections to one another are depicted. Cultures are maintained in control sleeves that may maintain temperature and stirring and may serve as a mount for electronic components which control and measure culture. A sleeve unit can typically be repeated and scaled to yield an array of arbitrary size. A corresponding pump array manages liquid dilutions through input of fresh media and efflux of old culture. The system is managed by a set of custom electronic and microcontroller boards that connect wetware components to a PC, which runs the control software for the system. The large quantities of data collected from experiments can be stored on a local server and uploaded to a cloud for streaming to remote devices.

FIGS. 2A-2C. Antibiotic pressure gradient experiment where a DIY CC system is used to evolve multi-antibiotic resistant E. coli. FIG. 2A. Measuring growth rate. A CC system was configured to run as a turbidostat, where cultures are diluted by a defined volume when they reach a target OD. Growth rate for a culture is measured by computing the first order derivative for the period of growth between dilution cycles. FIG. 2B. Pressure gradient algorithm. Growth rate (upper curve) is continuously assessed during an evolution experiment, and antibiotic concentration (lower curve) is increased when fitness recovers to that of WT. FIG. 2C. Time course for a pressure gradient evolution experiment. A WT E. coli strain was evolved under increasing concentrations of three clinically relevant antibiotics.

FIGS. 3A-3C. CC platforms provide insights into how to engineer circuits which don't impose a fitness burden. FIG. 3A. Schematic depicting the makeup of relevant two-node yeast genetic circuits. The circuits consist of an inducible promoter driving expression of a transcriptional activator that, in turn, activates expression of a GFP reporter gene. FIG. 3B. Comparison of growth rates of WT yeast and yeast transformed with a circuit or an insulated circuit as depicted in (FIG. 3A). Growth rates were measured on a CC system configured to run as a turbidostat. FIG. 3C. Comparisons of GFP expression for turbidostat-grown strains to that of their respective starter strains.

FIGS. 4A-4E. eVOLVER: an integrated framework for high-throughput, automated cell culture. FIG. 4A. Understanding how cellular phenotypes arise from multidimensional selection gradients requires multi-parameter control of culture conditions. FIG. 4B. Growth fitness experiments face a tradeoff between precision control of culture conditions and throughput. eVOLVER enables reliable scaling along both axes. FIG. 4C. eVOLVER hardware, fluidic, and software modules. System design is modular and synergistic. Left: eVOLVER is designed to scale to high-throughput. Center Top: Smart Sleeve unit. Smart Sleeves integrate sensors and actuators needed to measure and control parameters of individual cultures. Center Bottom: eVOLVER fluidic manipulation system (peristaltic pumps or millifluidic devices) controls movement of media and culture within the system. Right: A modular, scalable hardware architecture interfaces with Smart Sleeve and fluidic modules to achieve individually addressable, real-time culture control. The hardware functions as a bidirectional relay, streaming live data (via Raspberry Pi) collected from each Smart Sleeve to the external computing infrastructure running control software (written in Python). This software records and processes data and returns commands to the hardware in order to update culture parameters. System customization can be achieved by swapping fluidic handling devices, adding new parameter control modules, or programming new feedback control routines between culture and software. FIG. 4D. 16-culture eVOLVER base unit. Fluidics (media input, waste output) are physically separated from the electronics. The base unit can be cloned and parallelized to increase experimental throughput. FIG. 4E. eVOLVER hardware architecture. Smart Sleeves communicate with electronics module via a motherboard. Control modules, which control single parameters across for all Smart Sleeves within a 16-culture unit, are composed of Arduino-connected control boards occupying motherboard S/A slots. Arduinos are programmed to interpret and respond to serial commands from the Raspberry Pi, which communicates with software run on a user's computer or server.

FIGS. 5A-5C. Design and performance of eVOLVER modules. FIG. 5A. Generalizable configuration of Smart Sleeves for continuous culture; control of fluidic input/output, optical density, temperature, and stir rate. Left: Smart Sleeves are designed to accommodate 40 mL autoclavable borosilicate glass vials. Efflux straw length determines culture volume. Center: Smart Sleeve integrated electronic components. LED/photodiode sensor pairs perform OD₆₀₀ readings. Thermistors and heaters attached to a machined aluminum tube maintain PID temperature control. Magnet-attached computer fans rotate stir bars inside the vials. Components are wired to a PCB and mounted on an inexpensive 3D printed chassis. Individual sleeves cost ˜$25 and can be assembled in ˜10 minutes. Right: Specifications of Smart Sleeve parameters: optical density, temperature, and stirring. Device measurement precision varies with experimental conditions (e.g. cell type, room temperature) but can be adjusted to achieve necessary precision and range (e.g. tuning temperature PID constants or filtering OD measurements). Reported values are typical for experiments described in FIGS. 6A-6C and FIGS. 7A-7D. Calibration may be performed as often as desired, though settings are largely invariant over thousands of hours of use. FIG. 5B. “Basic” fluidic handling in eVOLVER utilizes pumps with fixed flow rates of ˜1 musec and can be actuated with a precision of ˜100 ms. FIG. 5C. Millifluidic multiplexing devices enable novel, customized liquid routing. Devices are fabricated by bonding a silicone membrane between two plastic layers with laser-etched flow channels. Integrated pneumatic valves actuate on the membrane to direct fluidic routing from media input to output ports (to or from vials).

FIGS. 6A-6C. High-throughput experimental evolution across a multidimensional selection space. FIG. 6A. Programming eVOLVER to maintain culture density selection routines during yeast evolution. Left: eVOLVER was configured to maintain cultures within defined density niches using a feedback between OD measurements and dilution events (turbidostat function). Right: Representative growth traces for yeast (Saccharomyces cerevisiae FL100) cultures growing under wide and narrow density niches. For each culture, the programmed OD window determines population size, and the consequent dilution rate and diauxic shift frequency. FIG. 6B. Parallel evolution of 78 yeast populations in distinct density niches. Culture OD traces are shown for populations evolved for 500 h in density windows with varied lower (0.05-0.6) and upper (0.1-0.65) OD thresholds. Lower right: Heat map of the estimated genome replication events for the 78 populations. Values were calculated by multiplying average number of cells by the number of doublings, both estimated through segmentation of the OD trace. FIG. 6C. Fitness distributions of evolved strains. Three clones from each evolved population were competed against the ancestral strain under low-density (OD 0.05-0.15, top) and high-density (OD 0.60-0.65, bottom) growth regimes. Right: Heat maps for mean fitness change relative to the ancestor (top) and ranked fitness with standard error bars representing competitive fitness for each clone (bottom).

FIGS. 7A-7D. Genome scale library fitness under temporally varying selection pressure. FIG. 7A. Programming temporally varying temperature regimes. Left: eVOLVER configuration for conducting turbidostat experiments (OD window: 0.15-0.2) under fluctuating temperature stress. Middle: Snapshot of temperature waveform alternating between 30° C. and 39° C. on a 6 h period, and corresponding culture growth rate. Right: Parallel cultures of pooled YKO collection were grown. Selection-based enrichment of library members was quantified at various timepoints using next-generation sequencing. FIG. 7B. Full set of dynamic temperature regimes. Temperature magnitudes (33° C., 36° C., 39° C. or 42° C.) were varied against periods (2 h, 6 h, or 48 h, or a constant step), and run against a 30° C. control culture. Recorded temperature is plotted with culture growth rates calculated between dilutions. FIG. 7C. Mapping fitness of library members to dynamic selection space. Left: For each library member, fitness heat maps were generated in each selection regime, and used to calculate weighted fitness centroids within temperature magnitude/frequency coordinate space. Right: Scatter plot of fitness centroids for the full library. FIG. 7D. Validation of library selection. Four strains with distinct profiles were chosen for verification and competed against a neutral control strain (ΔHO) under four different temporal selection regimes. Population ratios were measured using quantitative PCR.

FIGS. 8A-8C. Integrated millifluidic devices enable scaling of complex fluidic manipulation. FIG. 8A. Demonstrating dynamic media mixing in continuous culture. Left: eVOLVER program for maintaining cells in turbidostat mode using millifluidic device to mix and dispense appropriate dilution volumes. A yeast galactose-inducible reporter (pGAL1-mKate2) was used to validate the device by maintaining cultures in turbidostat mode at different ratios of glucose and galactose. Center: Any combination of seven media inputs can be mixed and dispensed into any of the 16 culture vessels. Right: Reporter induction (by population percentage) for 16 cultures containing different glucose:galactose ratios, as measured by flow cytometry. FIG. 8B. Preventing biofilm formation with automated vial-to-vial transfers. Left: A millifluidic device can enable inter-culture transfers between any of the 16 cultures. Center: Serratia marcescens cultures were maintained in turbidostat mode, with culture transfer events triggered every 8 h. Right: A culture maintained in a single vessel forms a thick biofilm after 14 h, while automated transfer prevents visible biofilm formation. FIG. 8C. Using millifluidic devices to automate yeast mating. Left: Haploid strains containing opposite mating types are maintained as turbidostat cultures under antifungal selection. Vial-to-vial transfers are triggered by growth rate feedback control, used to sample haploids and form diploids within the device using an automated mating protocol. Center: Growth rate of haploid cells evolved under cyclohexamide (CHX, 0.2 ug/mL, purple) or ketoconazole (KETO, 6 ug/mL, orange) selection was monitored continuously following drug exposure. Once growth rates of either drug-evolved culture equals 50% of the wild-type growth rate under no selection, automated mating and transfer is carried out. This was performed at two timepoints: t₁=68.7 h and t₂=98.1 h. Right: Antifungal resistance was assayed for recovered haploid and diploid populations. Contours correspond to an antifungal concentration range in which at least five generations of growth were observed in 24 h (on average).

FIGS. 9A-9C. Summary of eVOLVER hardware infrastructure. FIG. 9A. Electrical connections from Smart Sleeve to Motherboard: Sensors integrated into each sleeve interface with control elements on the Motherboard via a pluggable 14-pin ribbon cable (left). The connections from the cable are split and routed to the seven sensor/actuator slots (SA slots) to interface with the appropriate control circuit and microcontroller (right). FIG. 9B. Hardware configuration used for experiments: SA slots 1 to 5 are populated with components to control stirring, temperature, and optical density (see FIGS. 12A-12C, FIGS. 13A-13B, and FIGS. 15A-15B, respectively). Two SA slots are left open for customization. FIG. 9C. Photographs of Smart Sleeve and custom parameter boards. The Smart Sleeve disconnected from the Motherboard (left). A ribbon cable connects the Smart Sleeve to Motherboard (center). Printed circuit boards with the appropriate footprint can be plugged into SA slots for control/measurement of Smart Sleeve components (right).

FIGS. 10A-10B. Catalog of electronic boards. FIG. 10A. Core eVOLVER electronic boards: A Raspberry Pi, a small Linux board with RS485 shield plugged in for serial communication with Arduinos (upper left). 16-channel Motherboard with 5 (of 7) SA slots filled (upper right, see also FIGS. 9A-9C). Auxiliary board used for control of 48 fluidic control elements (e.g. peristaltic pump, solenoid valves) (lower left). Arduino microcontroller (32-bit/48 MHz ARM) pluggable into the Motherboard for control of SA slots (lower center). RS485 Board enabling serial communication between the Arduinos and Raspberry Pi (lower right). FIG. 10B. Customizable eVOLVER electronic boards: The 16-channel analog to digital converter board (ADC) is used to measure temperature or photodiode values simultaneously across all vials (left). The 16-channel pulse width modulation board (PWM) amplifies a 3.3V signal from the Arduino to the required voltage for control of motors, solenoids, or LEDs (center). These two types of PCBs are plugged into the Motherboard at SA slots to control sensors and actuators on the smart sleeve. Components of the smart sleeve are mounted on the CMB and then connected to the Motherboard (right) (see FIGS. 9A-9C).

FIG. 11. Network architecture of eVOLVER platform. Cloud framework enables live, remote visualization of experiments. A user programmable Python script controls an eVOLVER unit and streams collected data to the cloud. A single computer can handle many concurrently running Python scripts and thus many eVOLVER units. Raspberry Pi enables eVOLVER to be controlled remotely and parallelized. The Raspberry Pi in each eVOLVER unit has an application program interface (API) by which the lab computer (or any computer on the network) can query and record the status of the experiment. Based on the Python script running, the lab computer can then send configuration changes or commands to the Raspberry Pi and change any experimental parameter on that eVOLVER unit. This is then carried out internally via RS485 serial communication between the Raspberry Pi and the Arduinos.

FIGS. 12A-12C. Individually controllable stirring utilizing DIY parts. FIG. 12A. Photographs of eVOLVER stirring components. A 30 mm×30 mm computer fan affixed with neodymium magnets actuates stirring in the eVOLVER smart sleeve (left). Two ⅛″ acrylic sheets are used to space the magnets from the glass vial. The 3D printed part and CMB are fastened with screws (center, right). Electric leads are connected to the CMB with a screw terminal. FIG. 12B. Schematic of system design for eVOLVER stirring module. The computer fan spins a stir bar (20 mm×3 mm, PTFE coated) within a glass vial (28 mm×95 mm, borosilicate) (left). The Arduino interprets the serial command from the Raspberry Pi, amplifies the signal with the PWM board, and applies a 12V signal to the motor (right). The stir rate is determined by the ratio of pulsing the fan ON and OFF. FIG. 12C. Stir rates can be roughly calibrated by using a smartphone camera recording at ≥240 frames per second. Calibration curve shown is for a single Smart Sleeve. Stir rate was calculated multiple times in a five second window, with error bars depiction standard deviation of these measurements. Rotations per minute varies with different types of stir bars and volume of liquid in the vessel due to drag. Stir rates remain stable after 3 weeks of continuous use.

FIGS. 13A-13B. Individually controllable temperature achieved by feedback between thermometer and heaters integrated in the Smart Sleeve. FIG. 13A. Photographs of eVOLVER temperature components. A temperature-sensitive resistor, or thermistor, with a compact form factor, 25 mm×3.6 mm (left). Sensor integrated into Smart Sleeve in between the 3D printed part and spray painted aluminum tube (center). Two heaters are screwed onto the aluminum piece and all components are soldered onto the CMB (right). FIG. 13B. Schematic of system design for eVOLVER temperature module. The resistive heaters and thermistor are integrated into the Smart Sleeve and interface with PWM and ADC boards at SA slots 2 and 3, respectively. Arduino 2 manages both boards and interprets the desired temperature settings and responds with the current temperature (right). The temperature is maintained with a PID controller programmed into the Arduino. The controller interprets the input from the ADC board in slot 3 to determine the output of the PWM board in slot 2.

FIGS. 14A-14C. Temperature control characteristics in eVOLVER Smart Sleeves. FIG. 14A. Temperature calibration curves. Top: A thermocouple was used to measure the temperature at different thermistor readings. The points were fit with a line and all temperature measurements in the experiment were calculated based on the fitted line. Bottom: Recalibration, after a year of use, demonstrates stable and robust temperature control in Smart Sleeves. FIG. 14B. Temperature offset between aluminum sleeve and liquid. To measure the temperature offset during dynamic temperature changes, the integrated thermistor (upper left) and a thermocouple (lower left) simultaneously recorded temperature at two different locations during a square wave (right). FIG. 14C. Impact of temperature changes on optical density readings. Optical density calibration curves for yeast cultures (see FIGS. 16A-16C) were generated at three different temperatures, and verified separately by OD₆₀₀ spectrophotometer readings (left: curves from top to bottom are 35° C., 42° C., and 25° C.). To characterize temperature-induced OD offset without cells, evaporated milk was used to generate another set of calibration curves at different temperatures (right: curves from top to bottom are 40° C., 30° C., 35° C., and 25° C.).

FIGS. 15A-15B. IR LED-photodiode pair integrated in each Smart Sleeve enables individual monitoring of optical density. FIG. 15A. CAD drawing and photographs of a 3D printed part for housing optical parts. Designed on CAD software, printed parts housing the IR LED and photodiode are customized for 135° offset to maximize scattered light (left). Completed part printed from CAD file (center). CMB assembled with mounted LED and photodiode via screw terminals (right). FIG. 15B. Schematic of system design for eVOLVER optical density module. The IR LED (SA slot 4) and photodiode (SA slot 5) are integrated into the Smart Sleeve (left). A resistor is placed on the Smart Sleeve to limit current through the LED. A turbidity measurement is triggered by a serial command from the Raspberry Pi, and consequently, the Arduino responds with the current optical density measurements (right). The Arduino coordinates the timing when the LED flashes ON and the photodiode starts collecting measurements.

FIGS. 16A-16C. Optical density calibration and growth characterization. FIG. 16A. Optical density calibration curves. Optical density is measured by a 900 nm LED-diode pair (see FIGS. 15A-15B) and calibrated to an OD₆₀₀ measurement performed on a Spectramax M5 using 300 uL of media in a 96-well flat bottom plate. The calibration curve is fitted with a sigmoidal function. All optical density measurements in the experiments are calculated based on the fitted calibration curve for each Smart Sleeve. Sensitivity of OD measurements can be tuned by swapping the photodiode resistor. Top: A larger photodiode resistance at a lower LED intensity (2125 a.u.) gives a larger dynamic range, robust after 4 months of use. Bottom: A smaller photodiode resistor at a higher LED power gives a smaller dynamic range, but with more precision. This setting is also robust over time (1 year of use). Both traces are representative of a typical Smart Sleeve. FIG. 16B. Comparison of cell growth in flask vs Smart Sleeve. Comparison of yeast cells grown in flasks in a shaking incubator with cells grown in SDC in 18 different Smart Sleeves across 6 different eVOLVER systems (left). FIG. 16C. Comparison of cell growth across Smart Sleeves. We characterized variability of yeast growth across 96 Smart Sleeves (6 different eVOLVER platforms). Traces were aligned at 0.2 OD before plotting in order to normalize for different lag phases.

FIGS. 17A-17B. Modular fluidic control system for the eVOLVER platform. FIG. 17A. On left, hardware for fluidic control. The Auxiliary Board enables one Arduino to independently and simultaneously control 48 fluidic elements (e.g. pumps and valves) via three PWM boards. On right, schematic of system design for basic fluidic control. Serial commands from the Raspberry Pi are sent to the Motherboard and Auxiliary board on the same RS485 communication line. The Auxiliary board interprets the appropriate serial commands and actuates specific pumps for fluids to be metered in and out of a target smart sleeve. FIG. 17B. Interchangeable fluidic systems in the eVOLVER platform. Using the same serial communication and electronic hardware, the peristaltic pump array can be interchanged with other fluidic control elements, in this case, banks of solenoid valves used to control fluid routing in integrated millifluidic devices (see Example 15).

FIGS. 18A-18B. Arrayed peristaltic pumps for eVOLVER basic fluidic control. FIG. 18A. Photograph and calibration curve of a 16-unit peristaltic pump array. Each pump is wired (12V & GND) to the corresponding slot on the two 16-pin breakout boards. A ribbon cable connects the pump array to the Auxiliary board. For a single input turbidostat unit, two such arrays are used, one for influx and one for efflux. A linear calibration curve was created for each pump, taken from three technical replicates at four different pump durations. FIG. 18B. Flow rate measurements before and after an experiment demonstrates robustness of peristaltic pumps. During the experiment, each pump had a cumulative ON time of over 3,000 seconds (˜3 L of media).

FIGS. 19A-19C. Millifluidic devices featuring integrated pneumatic valves. FIG. 19A. Characteristics of macro pneumatic valves. A silicone rubber layer is sandwiched between two PETG plastic layers to form disposable, pneumatically-valved millifluidic devices (left). Valve layouts and fluidic paths can be designed with any vector-based CAD software, patterned with a laser cutter, and bonded with adhesive (upper right). The entire process, from CAD to completed device, can be done in 3 hours. Pneumatic valves and devices can be daisy chained together for improved scalability (lower right). FIG. 19B. Integrated millifluidic devices as fluidic modules. Completed devices are transparent, disposable, and patterned with a laser cutter (left). Fluidic routing and valving can be customized to form specialized fluidic modules (center). These modules can be connected in various ways to enable complex fluidic functions (right). FIG. 19C. Photograph of 16-channel multiplexer device, with fluidic lines (clear) and pneumatic lines (blue). Thread-to-barbed plastic connectors can be fastened onto the millifluidic device to interface with standard fluidic components.

FIG. 20. Photographs of 16-vial eVOLVER base-unit using “basic” fluidic scheme.

FIGS. 21A-21B. Long term maintenance of E. coli in eVOLVER. FIG. 21A. Left, sample OD traces of E. coli cultures maintained in eVOLVER. Observed noise is typical of clumpy bacterial cultures (see FIG. 22 for yeast OD trace) and does not affect average dilution rates as data is smoothed prior to triggering dilution events. Right, initial growth rate measurements for 8 turbidostat cultures. Over the first 48 hours, 8 E. coli cultures exhibit similar growth rates in M9 minimal media+0.4% glucose. Growth rates are calculated in segments of growth between each dilution event. FIG. 21B. eVOLVER is robust over long term culture. No hardware or software rashes occurred over a 250-hour experiment constituting over 200 generations of continuous exponential growth. Data collection was halted in periods where experiment was paused. Pausing was done intentionally for routine procedures (e.g. visual inspection, manual vial transfers) that limit selection for biofilm in bacterial cultures. These pauses account for a negligible portion of experimental time.

FIG. 22. Optical density trace with limiting glucose exhibits diauxic shift. Optical density traces measured from eVOLVER smart sleeves for the FL100 yeast strain grown in SC medium supplemented with 2% (left curve) or 0.06% (right curve) glucose.

FIG. 23. Growth rates across all 78 conditions during density dependent evolution. Calculated growth rates (h⁻¹) from optical density traces during the course of evolution. Growth rate calculations were made after every dilution event based on the number of population doublings completed since the last event divided by the length of time between dilutions.

FIG. 24. Measured evolutionary parameters during density dependent evolution. Left: Mean optical density during over the course of evolution experiment. Average densities skew lower than set point for a few narrow high-density conditions in which required dilutions were near the dose resolution of the pump. Middle: Mean growth rate. From growth rates measured over the course of the evolution experiment (FIG. 23), we calculated the mean growth rate, depict as a heat map (upper) and a ranked plot with standard deviation (lower). Right: Total calculated genome replication events. The average number of cells was calculated from mean optical density, based on a conversion rate of 10⁷ cells/mL for an OD 1.0 culture. Number of doublings were calculated based on the mean growth rate (from middle). Replication events were calculated by multiplying average number of cells with the number of doublings during the experiment. Doubling time and average OD listed for conditions at the limits. Part of right is reproduced in FIG. 6B.

FIGS. 25A-25D. Identifying correlations between fitness measurements and evolutionary parameters via k-means clustering. FIG. 25A. K-means clustering on low- and high-density fitness measurements: cluster 1, marked with “x” on the left; cluster 2, marked with “x” on the right; and cluster 3, marked with “x” on the top. Cluster centroids are reproducible for n=3 clusters, but not for higher n. FIG. 25B Clustering reveals three distinct groups: low-density specialists (cluster 2), high-density specialists (cluster 3), and the reminder exhibiting low fitness in both niches (cluster 1) (bars proceed from left to right as cluster 1, cluster 2, and cluster 3). FIG. 25C. Mapping the three clusters back to the evolutionary niches. FIG. 25D. Statistical analysis reveals significant differences between clusters (bars proceed from left to right as cluster 1, cluster 2, and cluster 3). Student's t-tests on imposed culture conditions and recorded evolutionary parameters of clusters indicate significant differences in the evolutionary conditions in which clustered colonies were derived.

FIG. 26A-26B. Design of primers for enumerating yeast deletion library members by qPCR or Illumina sequencing. FIG. 26A. qPCR amplicon design. For qPCR, universal reverse primer prCM314 (targeting a sequence from the deletion cassette downstream of the barcode) was paired with a context specific primer for each particular gene, usually a subsection of the “up45” homology region originally used to create the deletion library (Winzeler E. A., et al., Science 285, 901-906 (1999)). A control amplicon targeted two universal regions of the deletion cassette, piCM313 binding upstream of the barcode, and primer prCM317 binding in the KanMX resistance marker. Quantitative PCR was used to assay the relative quantity of a specific knockout strain of interest in a genomic DNA extract sample by comparing the specific amplicon to the control amplicon. FIG. 26B. Illumina library preparation. Illumina library preparation was performed with PCR in two steps. In the first step, universal primers prCM313 and prCM314 were used to extract and amplify all barcodes from a genomic DNA sample. In the second step, an 8-bp “timepoint” index and the i5 Illumina adapter sequence were attached using one of four unique forward extension primers, and an 8-bp “vial” index (yellow) and the i7 Illumina adapter sequence using one of sixteen unique reverse extension primers.

FIG. 27. Summary of raw sequencing counts of yeast knockout library. 244 million sequencing reads from an Illumina NextSeq run were assigned to one of 64 index pairs corresponding to vial and timepoint, revealing that sufficient data for frequency analysis was collected for 60 out of the 64 samples.

FIG. 28. Frequency analysis of strains present in each vial at initial and final timepoints of the pooled YKO library screen. We divided the number of barcodes reads assigned to each strain from the 5149 library members by the total number of reads assigned to each index pair, to determine a frequency for each mutant in a sample. These frequencies are plotted for the timepoints taken at Day 0 (black) and Day 6 (blue). Red line indicates the frequency to be expected if all library members were equally represented. For library members not detected in a Day 6 timepoint sample, were assigned a frequency of 10⁻⁸ for plotting. Library members that were missing at the Day 0 timepoint were excluded from plotting. Note wider frequency distributions at Day 6, in which a few members increase in frequency, but many members decrease in frequency, often by orders of magnitude, indicating specific enrichment for each condition. The 36° C./step condition exhibits missing members and inflated frequencies due to insufficient read depth (see FIG. 27). The 42° C./step data was excluded from analysis due to insufficient growth.

FIG. 29. Fitness centroids of high-performing strains in each vial correlate with selection conditions. To verify the centroid analysis metric for determining maximally fit strains across each condition (see FIG. 7C), 100 high-performing members from each condition were highlighted on the centroid distribution map. High-performing members were defined as those with the largest arithmetic difference in frequency between initial and final timepoints (i.e. freq_{Day 6}−freq_{Day 0}). The centroids of high-performing members clustered in a manner that correlates with the condition in which they were selected, e.g. high performers from the 42° C./48 hr condition cluster in the lower right portion of the graph.

FIGS. 30A-30C. Identifying library members with fitness centroids that significantly differ from population mean. FIG. 30A. Determination of significantly shifted library members. We considered library members with fitness centroids >1 standard deviation from the population mean to be significantly shifted. FIG. 30B. Highlighting significantly shifted library members that share annotated functions of interest. Fitness centroid distribution is reproduced from FIG. 7C, with selected library members colored by annotation. FIG. 30C. Complete list of library members with fitness centroids significantly shifted along either temperature magnitude or frequency axes. Strains are listed beginning with the library member furthest from the population mean along denoted axis direction. Note that some strains are listed in two lists.

FIG. 31. Groups of deletion mutants with shared phenotype annotations exhibit shifted average fitness centroids. We calculated average centroids for subsets of the deletion collection grouped by shared phenotype annotations on the Saccharomyces Genome Database (Cherry J. M., et al., Nucleic Acids Res. 40. D700-D705 (2012)). Welch's t-test (two-tailed) was applied to identify phenotype annotations whose subset centroid was significantly shifted from the population mean along either the temperature axis or the frequency axis. Each significant phenotype annotation is listed alongside the calculated subset centroid, the arithmetic difference from the population mean, the significance p-value (scaled to correct for multiple hypotheses), and the number of library members belonging to the annotation group. Note that certain phenotype annotations have further sub-annotations (“Resistance to Chemicals” could be further sub-divided by chemical, “Competitive Fitness” could be further subdivided by media condition, etc.) but these sub-annotations were not considered in the present work.

FIG. 32. Identities of high-performing strains are shared between similar conditions. For each condition, 100 high-performing members were defined as those with the largest arithmetic difference in frequency between initial and final timepoints, (see FIG. 28). The overlap between each condition is quantified by tabulating the number of strains shared. For each condition, the results are plotted in a heat map corresponding to the temperature magnitude and temperature period, ranging from no overlapping strains to 100% overlap.

FIG. 33A-33C. Principle component analysis divides selection conditions by shared effect on library. Principle component analysis was applied to determine whether similar conditions generally selected for the same library members. FIG. 33A. Principle component analysis separates conditions into three clusters that correspond to distinct regions of temperature magnitude/frequency space. Left: Principle component analysis was applied to a cross correlation matrix between the 14 conditions with sufficient sequencing read depth. This separates the conditions across two axes. Right: Each cluster corresponds to a distinct region of temperature magnitude-frequency space: two high-temperature groups corresponding to high- and low-frequency, and a mild temperature group. FIG. 33B. Gene ontology terms linked to fitness defects in each group. Welch's t-statistic was used to identify subsets of library members with shared annotation and significant fitness defects in each PCA cluster. FIG. 33C. Clusters are reproducible at earlier timepoints. To determine stability of these clusters, we projected of the cross-correlation results from earlier timepoints onto the same axes calculated from the Day 6 data.

FIGS. 34A-34C. Control structure for millifluidic devices enables unique fluidic programs for each experiment. FIG. 34A. Control structure for custom millifluidic devices. Fluidic sub-routines are pre-loaded onto an Arduino in order to ensure rapid and robust transition between the many sequential tasks needed to perform fluidic tasks on a custom fluidic module. These sub-routines convert abstract commands (e.g. dilute vial 1 with media A) into sequential actuation of control elements, such as solenoids for valving, or peristaltic and syringe pumps for media metering. FIG. 34B. Logic diagram for dilution event. For routine turbidostat dilutions, a dilution event triggered by reaching a density threshold consists of three parts: 1) Open route from appropriate media input, pull fluid into syringe, repeat as necessary in order to mix medias (as in glucose/galactose ratio sensing experiment, see FIG. 8A), then dispense through demultiplexer route into vial. 2) Open route through multiplexer to run efflux from vial to waste. 3) Open media selector route to 10% bleach, ethanol, then sterile water, to sterilize and flush fluidic paths used during dilution event. FIG. 34C. Logic diagram for vial to vial transfers. Transfer of cells from a source vial to a target vial were triggered either by elapsed time for the biofilm prevention experiment (see FIG. 8B) or by a growth rate measurement above threshold value for the antifungal evolution experiment (see FIG. 8C). A transfer consists of four parts: 1) Open route from appropriate media input, pull fluid into syringe, dispense through demultiplexer route into source vial. 2) Open route through multiplexer to run efflux from vial to syringe to collect cells. 3) Dispense through demultiplexer route into target vial. 4) Open media selector route to 10% bleach, ethanol, then sterile water, to sterilize and flush entire device.

FIGS. 35A-35C. Schematics of devices used in the present work. FIG. 35A. Vial router devices. These device consists of a valves and paths to form a demultiplexer/multiplexer pair in order to route fluid to and from 8 vial; two such devices were used in each 16-vial experiment. FIG. 35B. 8 channel media selector. This device consists of an 8-input multiplexer to select a media input and route it to one of two vial router devices. Sequential syringe pump events permit mixing of media, used to mix a glucose media with a galactose media in this experiment (see FIG. 8A). FIG. 35C. Vial-to-vial transfer device. The device used in the biofilm and mating experiments has an expanded media selector with more inputs (including bleach, ethanol, and water for flushing) and alternative paths to route cells from the efflux lines of one vial into the influx lines of another (via the two vial router devices, as before).

FIGS. 36A-36C. Media mixing for glucose/galactose ratio sensing experiment. FIG. 36A. Fluidic routing for ratio sensing experiment. To mix media types, the syringe pump first pulls sequentially from the desired media inputs. The syringe pump then flushes the syringe content into the target vial and, consequently, rises any trace amounts of leftover media to waste with sugar-free control media and air. FIG. 36B. Estimating sugar concentrations in culture medium. Glucose and galactose solutions were labelled with blue and yellow food coloring, respectively; these solutions were then used to mix media in a 4-fold dilution series of each sugar type (1%, 0.25%, and 0.06375%). We measured the 6 resulting medias, and a sugar-free control, on a spectrophotometer, using absorbance at 630 nm and 430 nm to estimate the component sugar concentrations independently. Using the multiplexed media handler, yeast cultures were maintained in eVOLVER across 16 different combinations of the glucose and galactose medias, by dynamically mixing any two of the six medias with each other, or the sugar free media (resulting in final concentrations ranging from 0-0.5% of each sugar). Spectrophotometer readings were collected at regular timepoints over 16 hours to confirm that the multiplexed media handler could maintain particular media combinations over the course of an experiment, as depicted photographically in FIG. 36B. in heat maps in FIG. 36C.

FIG. 37. Average growth rate varies according to ratio of glucose and galactose. For the glucose/galactose ratio sensing experiment (see FIG. 8A), mean growth rate for each condition over the 36 h experiment was determined by tracking optical density in eVOLVER. It should be noted that no growth was observed in the absence of both sugars, as expected.

FIG. 38. Schematic of fluidic routing for vial-to-vial transfer device. Example fluidic schematic for transfer of cells from vial 3 (source) to vial 4 (target). The syringe pump first pulls from the desired media input, then flushes the syringe content into the source vial. Instead of pumping efflux to waste, the fluid and cells from the source vial are pulled into the syringe pump (upper). Next, the contents of the syringe pump are dispensed into the target vial (lower). Sterilization with bleach and ethanol are required after vial to vial transfers to prevent contamination across vials.

FIG. 39. Prevention of biofilm formation by automated vial-to-vial passaging. Left: Passaging scheme. We programmed the vial-to-vial transfer device in order to automatically passage cells every 8 hours from one culture to a vial containing fresh media (see FIG. 8B and FIG. 38). Right: Density traces during passaging. We applied this scheme to maintain a culture of S. marcescens in continuous eVOLVER culture over 48 h. Following each passage, bleach was automatically added to the old culture, arresting growth. Every 24 h, these spent vials were replaced with new vials and filled with fresh media, indicated by red arrow.

FIGS. 40A-40B. Logic diagram for parallel evolution and mating of yeast. FIG. 40A. Logic for parallel evolution. Evolution was carried out in two selection vials run in turbidostat mode, supplied with antifungal media. When a selection vial recovered to 50% of its original growth rate, a timepoint sample was taken: three vials were inoculated, one with a cells, the second with a cells, and the third with both, to form diploids. In these vials, stirring was stopped once cells reached high density, in order to promote mating in the mixed vial. FIG. 40B. Simplified fluidics scheme for each timepoint. Three timepoints were taken: to at 16 h, t₁ at 68.7 h when the first selection vial recovered in growth rate, and t₂ at 98.1 h when the second selection vial recovered in growth rate.

FIGS. 41A-41D. Strain descriptions, selection scheme, and verification of yeast mating protocol. FIG. 41A. Logic diagram for yeast mating protocol. Following inoculation with each haploid type, the co-culture was grown to high density (OD 0.8) which triggered feedback on stirring, turned off to allow cells to settle and facilitate mating. FIG. 41B. Genetic manipulations of yeast strains. Two haploid yeast strains were transformed using one of two constitutive fluorescent reporters carried along one of two different auxotrophic selection markers, such that diploids formed from these cells would contain both fluorescent reporters and may be isolated by growing cells in dual-dropout media. FIG. 41C. Optical density trace from yeast mating on-device. We carried out yeast mating in eVOLVER with a program that allowed cells to grow to OD 0.8 in a well-mixed culture at which point stirring is halted. Over the next 24-36 hours, cells settle to the bottom of the vial, leading to a transient increase in the density observed by the sensor, followed by a sustained decrease as the cells fall below the path of the detector. At such high density in static conditions, haploid cells have the opportunity to form diploids. FIG. 41D. Verification by flow cytometry. Cells from the aforementioned automated mating protocol were grown in dual-dropout media to select for diploids, then measured by flow cytometry. Diploids (expressing both fluorescent markers) were isolated with 95% purity in this manner, with haploids of each type (expressing a single fluorescent marker) persisting in small, equal quantities.

FIG. 42. Antifungal resistance in haploid and diploids populations sampled during parallel evolution and mating experiment. To assay resistance, we performed a variant MIC growth assay with combinations of the two antifungals on population samples from each timepoint. Cell populations from each automated timepoint (see FIG. 8C) were seeded at OD 0.01 across a concentration range of ketoconazole and cyclohexamide. Heatmaps depict the average change in OD600 for cells in each concentration following 24 h growth in a 96 well block.

FIG. 43. Growth rate of pooled diploids created during dual antifungal evolution experiment. As an alternative metric by which to measure the results of the dual evolution and mating experiment (see FIG. 8C and FIGS. 40A-40B), we employed eVOLVER to measure the density and calculate the growth rate of diploids from each timepoint in different drug conditions. Doubling time was computed over the OD 0.2-0.8 range for duplicate vials of diploids formed at the “to pre-drug” control timepoint (yBWOO3, left two bars in each set), the “t₁ CHX recovery” timepoint (yBW008, middle two bars in each set), and the “t₂ KETO recovery” timepoint (yBW009, right two bars in each set) (see FIG. 8C and FIGS. 40A-40B) grown in four media conditions: no drug (left set of bars), cyclohexamide, ketoconazole, and the two drugs in combination. For yBW003 control diploids, a doubling time was fit based on what little growth occurred during the 24 period, as these cultures did not reach OD 0.8.

FIG. 44. ERG3 sequence alignment reveals nonsense mutation. Alignment of ERG3 sequences from founder strain and three resistant clones isolated from the ketoconazole selection vial at t₂. Several grouped missense mutations at amino acids 57-59 are followed by a nonsense mutation at amino acid 60.

FIGS. 45A-45B. Programming and evaluating a chemostat dilution scheme in eVOLVER. FIG. 45A. Logic diagram for chemostat culture. To demonstrate that eVOLVER programs can carry out continual chemostat culture in a replicable manner, we inoculated 8 vials with E. coli, and maintained these cells under continual dilutions at 4 specified inter-pump periods, setting the death rate of each culture. FIG. 45B. Optical density traces. The optical density of each culture was tracked in order to determine if duplicate cultures reach similar culture densities.

### DETAILED DESCRIPTION

Others have reported the compositions of various DIY continuous culture (CC) systems (Toprak E., et. al., Nat. Genet. 44, 101-5(2012); (Acar M., et. al., Nat. Genet. 40, 471-75 (2008); https://depts.washington.edu/soslab/turbidostat/pmwiki; http://openwetware.org/wiki/Turbidostat). A typical DIY CC system is comprised of integrated wetware, hardware, and software modules that can be readily interchanged and reconfigured to perform a wide variety of long-term laboratory experiments. Maintenance of culture conditions (e.g., temperature, stirring speed, and media composition) can be automated over long periods. Such a system permits the user to collect multiple streams of data in real time, and algorithmically adjust culture conditions continuously.

DIY construction approaches can be easily scaled to high throughput. Moreover, CC systems are highly modular, and both device and software are relatively easy to reconfigure and operate. This modularity makes it possible for users to quickly elaborate on a system's capabilities as per the user's needs. In the past, most CC evolution experiments were restricted to low throughput, limiting the ability to assess the relationship between culture evolvability and either culture conditions or parental strain genotype. Scalability of DIY CC systems addresses this issue, as the effects of a variety of culture conditions on evolution can be tested simultaneously. Thus, DIY CC systems can be used for a number of applications with potential commercial relevance, including testing new antibiotic treatment regimes for resistance acquisition or testing communities of strains for their ability to complement or support each other's growth.

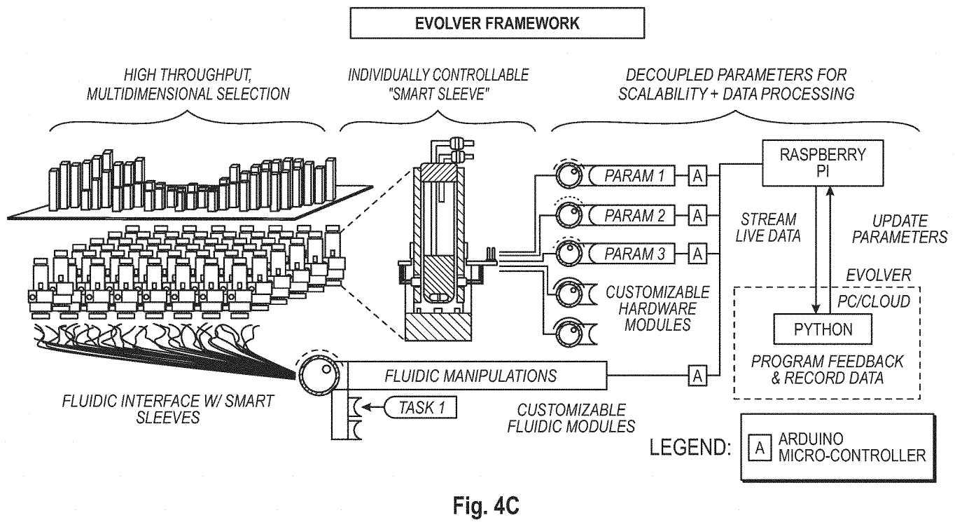

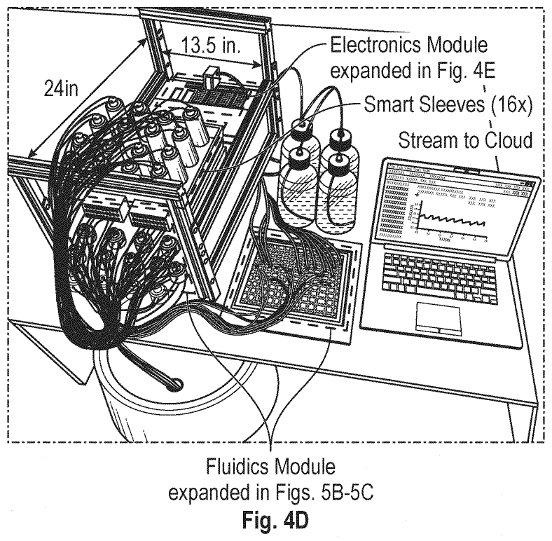

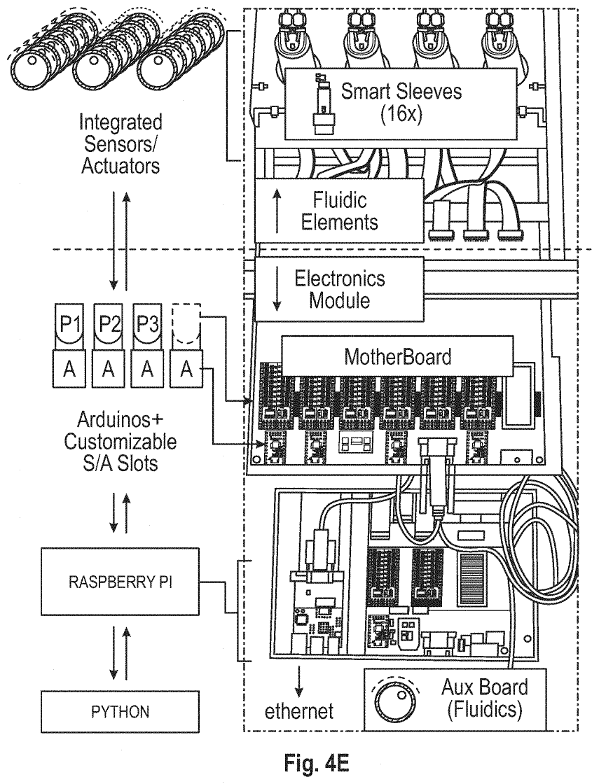

Biological organisms are embedded in complex, dynamically changing environments that shape their evolved phenotype (Elena S. F., et al., Nat. Rev. Genet. 4, 457-469 (2003); Nichols R. J., et al., Cell 144, 143-156 (2011); Nevozhay D., et al., PLoS Comput. Biol. 8, e1002480 (2012).) (FIG. 4A). An integrated understanding of how phenotype is encoded and adapted to specific selective pressures remains a central challenge for biological research. In the laboratory, experiments involving growth selection in liquid culture have been integral to studying the relationship between genotype and phenotypic fitness. These include functional genomic library screening/selection (Giaever G., et al., Nature 418, 387-391 (2002); Li Z., et al., Nat. Biotechnol. 29, 361-7 (2011)), characterization of natural and synthetic cellular systems (Zuleta I. A., et al., Nat. Methods 11, 443-8 (2014); Takahashi C. N., et al., ACS Synth. Biol. 4, 32-38 (2015); Feist A. M., et al., Nat. Rev. Microbiol. 7, 129-143 (2008)), and directed evolution to either study evolutionary processes (Lang G. I., et al., Nature 500, 571-574 (2013); Maddamsetti R., et al., Genetics 200, 619-31 (2015); Yona A. H., et al., Proc. Natl. Acad. Sci. U.S.A 109, 21010-5 (2012)) or evolve new biological function (Wang H. H., et al., Nature 460, 894-898 (2009); Esvelt K. M., et al., Nature 472, 499-503 (2011); Ravikumar A., et al., Nat. Chem. Biol. 10, 175-177 (2014); Crook N., et al., Nat. Commun. 7, 13051 (2016)). While identification of selected genotypes has dramatically improved with the advent of next generation sequencing, the ability to precisely and systematically control the conditions of selection has not improved in complimentary fashion, hampering understanding of how adaptive genotype is shaped by selective pressure (Bull A. T., J. Ind. Microbiol. Biotechnol. 37, 993-1021 (2010)).

An intrinsic challenge in designing growth selection experiments lies in balancing the tradeoff between control and throughput (FIG. 4B); while batch cultures maintained by serial passage permit parallel testing of many strains or cultures conditions, they are inherently discontinuous and offer limited temporal control over culture conditions (Gresham D. and Dunham M. J., Genomics 104, 399-405 (2014)). Conversely, automated cell growth systems can maintain constant growth rates under precisely defined conditions, but are difficult to parallelize due to cost, space-inefficiency, and design complexity (Bull A. T., J. Ind. Microbiol. Biotechnol. 37, 993-1021 (2010); Gresham D., and Dunham M. J., Genomics 104, 399-405 (2014); Piper M. D. W., et al., J. Biol. Chem. 277, 37001-37008 (2002)). A recent convergence of several open-source technologies-inexpensive additive manufacturing, do-it-yourself (DIY) software/hardware interface, and cloud computing—has enabled the in-lab fabrication of new custom laboratory platforms (Cressey D., Nature 544, 125-126 (2017); Kong D. S., et al., Nat. Biotechnol. 35, 523-529 (2017)). Various examples in automated cell culture include devices designed to perform automated dilution routines for exploring antibiotic resistance acquisition (Toprak E., et al., Nat. Genet. 44, 101-105 (2011)), or that implement design features such as real-time monitoring of bulk fluorescence (Takahashi C. N., et al., ACS Synth. Biol. 4, 32-38 (2015)), light-based feedback control of synthetic gene circuits (Milias-Argeitis A., et al., Nat. Commun. 7, 12546 (2016)), and chemostat parallelization (Hope E. A., et al., Genetics 206, 1153-1167 (2017)). Unfortunately, the single-purpose, ad hoc design of these systems limits their scalability and restricts their reconfiguration for other experimental purposes.

Here we present eVOLVER, a multi-objective, DIY platform that gives users complete freedom to define the parameters of automated culture growth experiments (e.g. temperature, culture density, media composition, etc.), and inexpensively scale them to an arbitrary size. The system is constructed using highly modular, open-source wetware, hardware, electronics and web-based software that can be rapidly reconfigured for virtually any type of automated growth experiment. eVOLVER can continuously control and monitor up to hundreds of individual cultures, collecting, assessing, and storing experimental data in real-time, for experiments of arbitrary timescale. The system permits facile programming of algorithmic culture ‘routines’, whereby live feedbacks between the growing culture and the system couple the status of a culture (e.g. high optical density (OD)) to its automated manipulation (e.g. dilution with fresh media). By combining this programmability with arbitrary throughput scaling, the system can be used for fine resolution exploration of fitness landscapes, or determination of phenotypic distribution along multidimensional environmental selection gradients.

We demonstrate the broad applicability of eVOLVER by configuring it to perform diverse growth and selection experiments. First, we conduct high-throughput experimental evolution on yeast populations using multi-dimensional selection criteria, scanning culture density space at fine resolution to assess adaptive outcomes. Next, by performing growth selection on a yeast knockout (YKO) library under temporally variable temperature stress regimes, we show that eVOLVER can be used to systematically explore the relationship between environmental fluctuations and adaptive phenotype. Finally, by integrating millifluidic multiplexing modules, we demonstrate that eVOLVER can carry out complex fluidic manipulations, dramatically extending the scope and range of possible growth and selection experiments.

As described herein, CC devices, in several different implementations, can serve as enabling technology for a number of under-explored areas in experimental evolution including selecting for novel functionality in wild-type microorganisms, improving engineered circuit stability by evolving both the circuit and host, testing and evolving stable multi-species communities, and testing engineered synthetic communities.

In some aspects, the disclosure relates to a continuous culture system that is configured for high-throughput culturing of microorganisms. As used herein the terms “microbial,” “microbe,” or “microorganism” all relate to microscopic living organisms including archaea, bacteria, fungi, protista, microbial mergers or symbionts, planarians (e.g., C. elegans), and suspensions of mammalian cells, plant cells, or insect cells.

In some aspects, the disclosure relates to a continuous culture system that is configured for high-throughput microbial evolution studies. Batch culture experiments are traditionally used for high-throughput laboratory evolution studies. Configuring a CC system to perform experiments at high-throughput offers a way to do continuous culture experiments at the same scale. While more technically challenging than performing batch culture, a continuous culture system allows for precise control and monitoring of growth conditions during an evolution experiment. By virtue of its scalability, a CC system permits the systematic exploration of the relationship between culture regime and adaptation. Antibiotic resistance experiments are one example of an application for this approach, as a CC system configured for high-throughput can be used to test the relationship between antibiotic resistance and different quantitative features of the stress ramp algorithm.

In some embodiments, a continuous culture system configuration comprises at least one stress ramp function that is overlaid on top of at least one culture fitness function, wherein the relationship between the at least one stress ramp function and the at least one fitness function responds to increased culture fitness with increased application of stress in real-time. As used herein, a “culture fitness function” refers to an output that is indicative of microbial growth or health. In some embodiments, the culture fitness function consists of one microbial fitness measurement. In other embodiments, the culture function comprises more than one fitness measurement. Examples of fitness measurements are provided below (e.g., turbidity and fluorescence). As used herein, the term “stress ramp function” refers to an input that applies stress on microbial growth or health. In some embodiments, the stress ramp function consists of one microbial stress. In other embodiments, the stress ramp function comprises more than one microbial stress. Examples of microbial stresses are provided below.

In some aspects, the disclosure relates to a continuous culture system that is configured for the testing of mutational stability of engineered circuit variants (e.g., assaying how long it takes for a circuit to inactivate or lose at least some portion of its function). A major focus of synthetic biology has been on engineering synthetic regulatory circuits to enable user-defined control of cellular function. Circuits engineered in E. coli, yeast, and other microorganisms often impose a fitness burden on their host cells and may be lost or mutated over time. Very little work has gone into engineering circuits that are either robust to mutation or minimize host-cell burden. By the same token, efforts to engineer strains that can accommodate circuits without mutating them have not been undertaken.

In some aspects, the disclosure relates to a continuous culture system that is configured to assay circuit stability by growing at least one microbial cell comprising at least one circuit (or circuit library) and then assessing mutations that accrue to either the at least one circuit or the genome of the host microbial cell. In some embodiments, the at least one microbial cell comprising at least one circuit (or circuit library) is grown under stress. By selecting for circuit variants or strain backgrounds that render the circuit resistant to inactivation or loss of function, engineering rules for circuit stability can be determined.

In some embodiments, the continuous culture system is configured as a turbidostat with fluorescence detection to measure circuit output and to track the loss (or gain) of circuit function over the course of an experiment. As used herein. “turbidostat” refers to a culture device that has feedback between the turbidity of the culture vessel and the dilution rate. In some embodiments, the circuit output is a detectable marker, such as the production of a fluorescent protein including, but not limited to, TagBFP, mTagBFP2, Azurite, EBFP2, mKalama1, Sirius, Sapphire, T-Sapphire, ECFP, Cerulean, SCFP3A, mTurquoise, mTurquoise2, monomeric Midoriishi-Cyan, TagCFP, mTFP1, EGFP, Emerald, Superfolder GFP, Monomeric Azami Green, TagGFP2, mUKG, mWasabi, Clover, mNeonGreen, EYFP, Citrine, Venus, SYFP2, TagYFP, Monomeric Kusabira-Orange, mKOκ, mKO2, mOrange, mOrange2, mRaspberry, mCherry, mStrawberry, mTangerine, tdTomato, TagRFP, TagRFP-T, mApple, mRuby, mRuby2, mPlum, HcRed-Tandem, mKate2, mNeptune, NirFP, TagRFP657, IFP1.4, or iRFP. In some embodiments, the circuit output is the production of a protein tagged with a fluorescent protein.

In some aspects, the disclosure relates to a continuous culture system that is configured to test and evolve stable multi-species communities. There is a growing research interest in understanding how microbial interspecies interactions give rise to stable communities. A properly configured CC system can be used to test the stability of community interactions in a long-term continuous growth setting. At high throughput, different combinations of species and variations in culture conditions can be tested for their ability to support community fitness and structure. In some embodiments, a CC system is configured as a turbidostat and used to culture species combinations in order to assess interaction stability and emergent community structure. In some embodiments, the fluidics of a CC system are configured to allow vial-to-vial culture transfer. In some embodiments, the continuous culture system enables the continuous mixing of individual cultured species into a community culture. As used herein, a “multi-species community” is a culture in which at least two independent microbe species co-exist. In some embodiments, the at least two independent microbe species are from the same kingdom, phylum, class, order, or genus. In some embodiments, the at least two independent microbe species are from a different kingdom, phylum, class, order, or genus.

In some aspects, the disclosure relates to a continuous culture system that is configured to engineer synthetic communities. In some embodiments, a continuous culture system is configured as a tool to facilitate the bottom-up construction of microbial communities from strains harboring engineered circuits that facilitate cell-cell interaction. In some embodiments, a continuous culture system is configured to test the stability of interactions between strains. In other embodiments, a continuous culture system is configured to generate continuous co-cultures through vial-to-vial transfer.

In some embodiments, the continuous culture system comprises integrated wetware (e.g., culture vessels and fluidics), hardware (e.g., electronics), and software (e.g., Python, Arduino C and Javascript) modules that can be readily interchanged and reconfigured. Some embodiments of the wetware, hardware and software modules are described in more detail elsewhere herein.

In some embodiments, the continuous culture system is configured for long-term continuous culture. As used herein, “long-term” refers to any prolonged period of time, including, but not limited to, one or more hours, one or more days, one or more weeks, one or more months, or one or more years.

In other aspects, the disclosure relates to a method of performing experimental evolution on at least one fluidic microbial culture in a continuous culture system. In some embodiments, at least one microbial culture is subjected to a dynamic environment, wherein the at least one microbial culture is exposed to a stress ramp function which is overlaid on top of a culture fitness function, and increasing the amount of stress applied to the at least one microbial culture in response to the increased fitness of the at least one microbial culture. In some embodiments, microbial fitness is calculated in real-time.

In some embodiments, microbial fitness is calculated through turbidity or fluorescence measurements.

In some embodiments, the stress applied to the at least one microbial culture is an antibiotic. In some embodiments, the stress applied to the at least one microbial culture is an antiseptic, including but not limited to, alcohol (e.g., ethanol), hydrogen peroxide, iodine, benzalkonium chloride, or boric acid. In other embodiments, the stress applied to the at least one microbial culture is an increase or decrease in temperature. In other embodiments, the stress applied to the at least one microbial culture is selected from the list comprising: growth under redox stress, growth under aerobic or anaerobic conditions, or growth under challenge from an infectious agent (e.g., a fungi, bacteria, phage or virus). In some embodiments, the stress applied to the at least one microbial culture is a nutrient stress. In some embodiments, the nutrient stress is a nutrient poor condition (i.e., the nutrient condition is insufficient to meet the microorganism's bioenergetics needs). In some embodiments, the nutrient stress is a nutrient rich condition (i.e., the nutrient condition exceeds the microorganism's bioenergetics needs). In some embodiments, the stress is irradiation. Examples of ionizing and non-ionizing irradiation are known to those having skill in the art and include, but are not limited to, alpha particles, beta particles, positrons, photons, charged nuclei, neutrons, gamma rays, X-rays, UV, infrared, microwaves, radio waves, or cosmic rays. In some embodiments, the stress is a change in pH. In some embodiments, the stress a metabolic stress (e.g., anabolic or catabolic). In some embodiments, the metabolic stress is an increase or decrease in gene expression or protein translation relative to the level of gene expression or protein translation in the absence of the metabolic stress. In some embodiments, the stress is a mechanical stress (e.g., increased or decreases in pressure, increased or decreases in vibration, increase or decreases in motion, etc.). In other embodiments, more than one stress is applied to the at least one microbial culture.

In some embodiments, at least one microbial culture evolves a novel functionality. In some embodiments, the novel functionality is altered stress tolerance, altered nutrient utilization, or altered metabolite production, in which “altered” means changed relative to the starting microbial culture. Stress tolerance includes tolerance to higher or lower temperatures, higher or lower concentrations of salts in the culture, higher or lower amounts of sugars or other carbon-based feedstocks in the culture, etc. Altered nutrient utilization includes altered rates (lower or higher) of nutrient utilization and/or utilization of different or additional nutrients. Altered metabolite production includes altered rates (lower or higher) of metabolite production and/or production of different or additional metabolites. In other aspects, the disclosure relates to a method of performing experimental evolution.

In some aspects, the disclosure relates to a method of culturing of microorganisms.

In some aspects, the disclosure relates to a method of testing the mutational stability of a microbial cell that comprises an engineered circuit. In some embodiments, the method relates to culturing at least one fluidic microbial culture in a continuous culture system and determining the time required for an engineered circuit to inactivate after subjecting a microbial cell to a dynamic environment, wherein the at least one microbial culture is exposed to a stress ramp function which is overlaid on top of a culture fitness function, and increasing the amount of stress applied to the at least one microbial culture in response to the increased fitness of the at least one microbial culture. As used herein, the term “inactivate” refers to a decrease in the output of an engineered circuit by at least about 20%, 25%, 30%, 40%. 50%, 60%, 70%, 75%. 80%, 90%, 95%. 99% or more than 99% relative to the output prior to application of the stress.

In some embodiments, microbial fitness is calculated in real-time. In some embodiments, the method evolves both the circuit and the microbial host cell. Engineered circuits, such as engineered gene circuits for expressing one or more outputs (such as proteins) in response to one or more signals, are known in the art.

In some aspects, the disclosure relates to a method of testing the stability of at least one multi-species microbial community. In some embodiments, the method relates to culturing at least one fluidic multi-species microbial culture in a continuous culture system and determining the fitness of each species independently after subjecting a microbial cell to a dynamic environment, wherein the at least one microbial culture is exposed to a stress ramp function which is overlaid on top of a culture fitness function, and increasing the amount of stress applied to the at least one microbial culture in response to the increased fitness of the at least one microbial culture. In some embodiments, microbial fitness is calculated in real-time.

In other aspects, the disclosure relates to a method of constructing a multi-species community. In some embodiments, the method relates to culturing a multi-species microbial community in a continuous culture system, wherein the multi-species community comprises microbial strains that comprise engineered circuits that facilitate cell-cell interactions. A multi-species microbial community can include two or more microbial strains (of which none, some or all may include engineered circuits), such as 2, 3, 4, 5, 6, 7, 8, 9, 10, or more microbial strains. In some embodiments, the multi-species microbial community is subjected to a dynamic environment, wherein the microbial community is exposed to a stress ramp function which is overlaid on top of a culture fitness function, and increasing the amount of stress applied to the microbial community in response to outputs from the engineered circuits. In some embodiments, the output is calculated in real-time.

### EXAMPLES

### Example 1. Customizability of DIY CC Systems

A typical scheme for such a system is depicted in FIG. 1. During evolution experiments, glassware containing live cultures may be housed in individual control sleeves (Toprak E., et. al., Nat. Genet. 44, 101-5(2012); https://depts.washington.edu/soslab/turbidostat/pmwiki; http://openwetware.org/wiki/Turbidostat). Cultures may be grown at ambient temperature (Toprak E., et. al., Nat. Genet. 44, 101-5(2012); (Acar M., et. al., Nat. Genet. 40, 471-75 (2008); https://depts.washington.edu/soslab/turbidostat/pmwiki), or may feature temperature regulation via a heated jacket mounted on the sleeve (http://openwetware.org/wiki/Turbidostat). Typically, a magnetic stirring mechanism is used to maintain aeration on each culture. Control sleeve designs vary considerably and may reflect users' specific experimental needs. For example, in addition to the jacket and fan, a CC configuration designed for turbidostat function might include a LED/detector photodiode pair for measuring optical density. Sleeve units are often compact and can be readily arrayed in a format permissible for high-throughput screening.

Media input and efflux from each culture can be maintained by an array of peristaltic pumps. CC systems often feature an electronics control layer—composed of commercially available data acquisition boards, microcontrollers (e.g., Arduino), and switch arrays—is used to control all fluidic and sleeve-associated functions. Microcontrollers and relays may be connected by USB to a laptop computer running control software. The software can be configured to specify control sleeve and fluidic settings for each individual culture. The laptop may be attached to a server that collects and stores experimental data. Plotted data can be streamed to a webpage allowing experiments to be observed remotely by either laptop mobile device.

The modular customizability most DIY CC systems enables the user to conduct a broad repertoire of evolution experiments. For example, in a simple implementation, chemostat experiments can be run by programming the fluidics module for steady-state media influx and efflux. When integrated with data collection form a control sleeve-mounted led/detector pair, a device can be used to perform turbidostat experiments. Depending on the type of experiment, a system can be configured to collect data from additional electronic component sources mounted on the control sleeve. For example, in addition to temperature and OD reading associated with a standard turbidostat, a light source and detector can be installed to collect fluorescence data if a user was interested in monitoring gene expression.

### Example 2. Design and Application of DIY CC Systems to Evolve Multi-Antibiotic Resistant E. coli

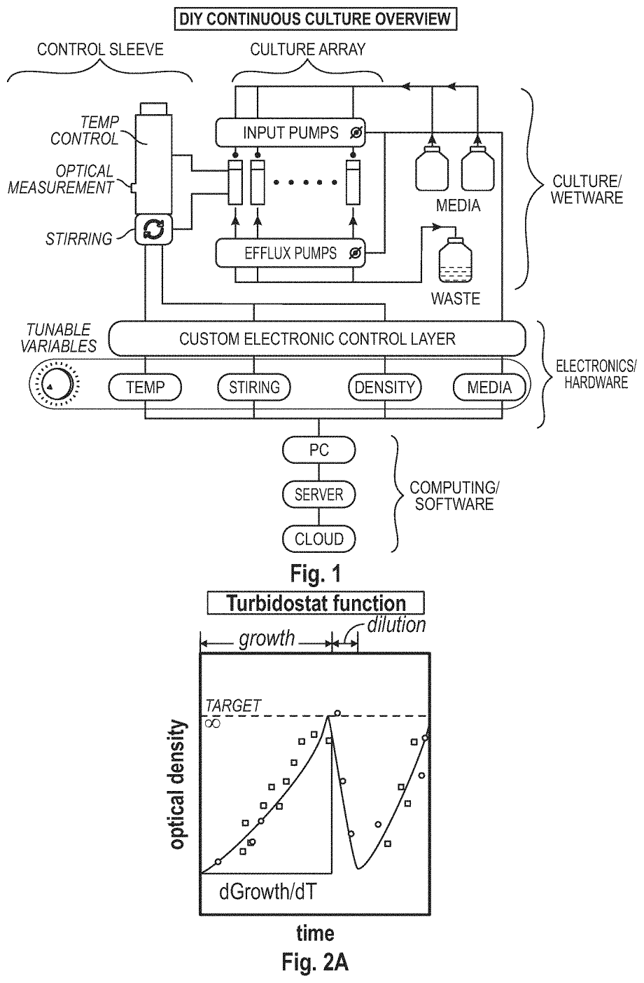

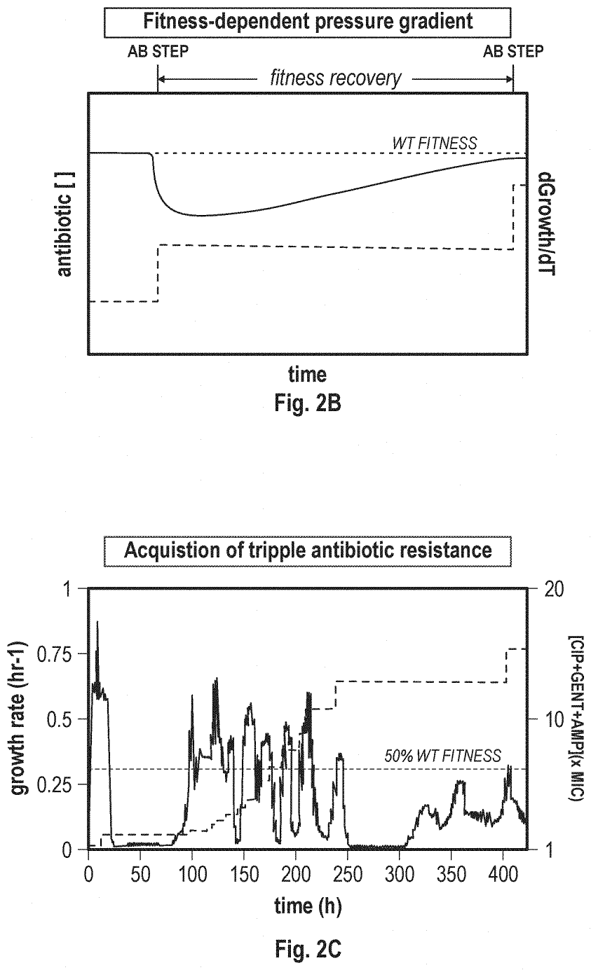

By integrating real-time data collection and processing with fluidic function, it is possible to specify dynamic culture environments with contingency-based stress regimes. For example, a stress ramp function can be overlaid on top of turbidostat function by including an additional set of input pumps to supply media containing a stress agent (e.g., antibiotics), and then gradually increasing the ratio of stress/no stress media added to the cultures over the course of the experiments. In FIG. 2, a cultures is evolved with a ramp algorithm in which stress is increased only after the culture's fitness (growth rate) has recovered from a previous increase (FIGS. 2A-2B). The results of such an experiment are shown in FIG. 2C where antibiotic concentration (CIP. GENT and AMP; upper curve at right border) and growth rates (lower curve at right border) are plotted for a cultures evolving for 425 hours.

### Example 3. Design and Application of DIY CC Systems to Improve Engineered Circuit Stability

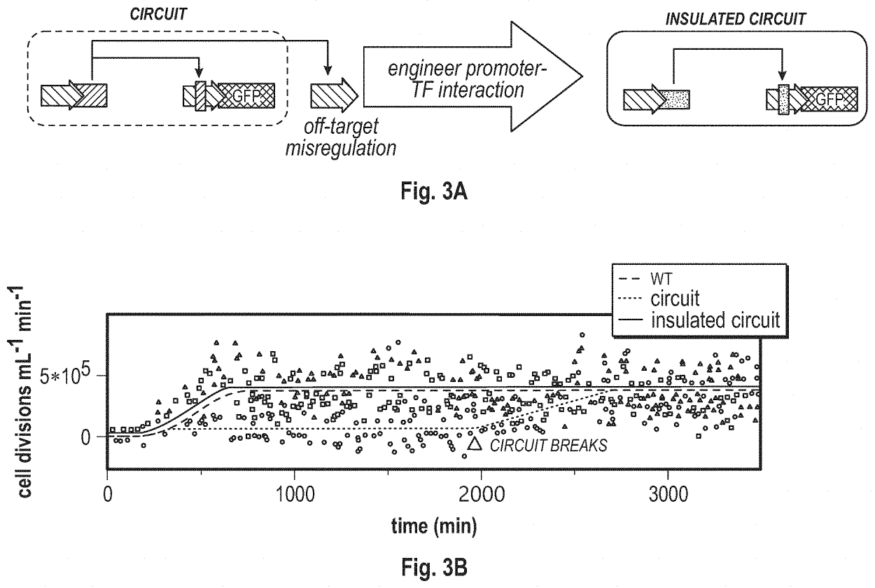

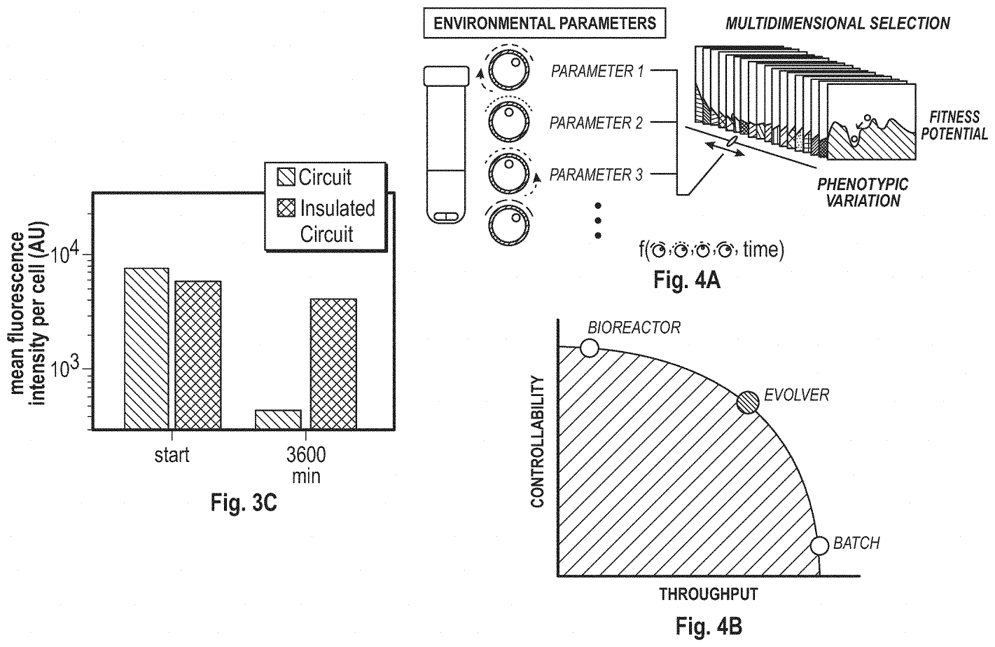

The introduction of engineered circuits into cells can result in a growth penalty through 1) placing a substantial metabolic load on the system or 2) misregulation of native function. Very little research has gone into understanding how to engineer circuits which do not impose a fitness burden. Here, a CC platform was used to test the stability of simple two-node circuits in yeast. The circuits consist of an inducible promoter driving expression of a transcriptional activator that, in turn, activates expression of a GFP reporter gene (FIG. 3A). Previous experiments, indicated that the expression of the activator resulted in sub-optimal growth. Misregulation of an off-target locus might account this fitness cost.

In order to insulate the circuit from this interaction, several aspects of the activator-promoter interaction were engineered at the reporter node. The ability of the insulated circuit to ameliorate the fitness defect was tested by growing each of the strains in media containing inducer, in a CC system configured to run as a turbidostat. As shown in FIG. 3B, growth rates for the insulated circuit were similar to that of untransformed yeast, while the uninsulated circuit showed significant growth rate penalty. However, after approximately 1950 minutes, the growth rate of the un-insulated circuit accelerated, rapidly attaining the same rate as the WT and insulated circuit. This suggested that the circuit had “broken” (i.e., it had shed its fitness cost) because it was no longer functioning. To test this, at the conclusion of the experiment (3600 minutes) GFP expression for turbidostat-gown strains were compared to that of their respective starter strains (FIG. 3C). While the insulated circuit showed essentially unchanged expression from the start of the experiment through to the end, the un-insulated circuit had lost its ability to inducibly transcribe GFP (probably during the observed shift in fitness).

### Example 4. Detailed Design of Continuous Culture System

Described here in detail is the continuous culture system that has been constructed to execute various experiments. The described implementation is specifically designed for doing pressure step turbidostat experiments that feature two media input lines, one efflux line, and vial-proximate instrumentation that monitors culture OD at a fixed stirring rate and temperature. Elaboration or rearrangement of the components to realize other experimental setups can be accomplished easily.

Overall Design:

The continuous culture system design features separate modular wetware (culture vessels and fluidics), hardware (DIY electronics) and software (Python, Arduino C and Javascript) layers that can be adjusted or upgraded individually. For example, the custom electronic layer controls each experimental dimension (e.g., temperature, fluidics, etc.) with separate Arduino microcontrollers. The design allows for upgrades that are made within each control module to not require restructuring of the entire platform. This allows for quick customization if new experimental capabilities are desired. Additionally, using Arduino microcontroller boards enable users to test their own customized sensors before large-scale integration into the full platform.

Wetware:

Evolution experiments are conducted in 40 mL glass vials fitted with custom machined Teflon tops. Segments of PEEK tubing are inserted into the tops and connect silicone tubing lines to the vials. Silicone tubing is attached to peristaltic pumps that push media into the vials from large volume (5 or 10 L) reservoirs, or pull culture out of vials and into a waste reservoir. Prior to running experiments, vials, media reservoirs, and lines are assembled and sterilized, and then connected together before loading media into the vials array.

Hardware:

Smart Sleeve:

During experiments, culture vials are housed in easily manufactured smart sleeve units. The units consist of 3D printed chassis which are used to mount an aluminum jacket for controlling temperature, and an inexpensive computer fan with attached magnets in order to spin stir bars inside culture vials. Each smart sleeve features a small circuit board that serves as a mount for connecting electronic components. Heating of the aluminum jacket is accomplished by heating resistors that directly contact the aluminum tubes, while temperature is measured with inexpensive thermistors. Optical density is measured using an inexpensive LED photodiode emitter/detector pair. Additional smart sleeve customizations include sensors that measure levels of dissolved O₂, CO₂, N₂, or other gases; sensors that measure redox potential; and/or emitters/detectors that excite and detect multiple wavelengths of light for optogenetics or fluorescence-based assays. The mounted boards gather signals for all components on a single sleeve into a ribbon cable that leads to a custom PCB motherboard.

Electronics:

The function of the motherboard is to take sets of signals from the smart sleeve array and sort them to the appropriate control and sensor boards. This setup enables plug and play of corresponding smart sleeve and board functions, with a single motherboard designed to route up to seven sensors/actuators to an array of 16-smart sleeves. Cable ribbons that correspond to each function run from the motherboard to each control board. The printed circuit boards were obtained from International Circuits Inc. (internationalcircuits.com).

Custom Arduino shields were designed for either reading and supplying voltages for several of the smart sleeve components. For reading analog signals, a simple voltage divider circuit board was constructed that connects to an Arduino Mega. While this board design is amenable to reading a variety of analog voltages, in this specific system's implementation the boards were used to obtain readings from the thermistors. This board can accommodate up to 16 channels and uses a 9 V power supply. To best make thermistor measurements, a 10 Ohm resistor was used, and no additional buffer or filter was required. This design is modular, and could be adapted to gather voltage data from other types of components. For OD measurements, two 8 channel National Instruments digital acquisition (DAQ) devices were used, which were connected to the motherboard via a ribbon cable and breakout board.

To regulate the power supplied to components, a shield for the Arduino Uno was designed that is able to perform PWM voltage control. This transistor control board uses TLC5940 PWM expansion chip to control an N-Channel MOSFET transistor array. One board can supply addressable voltage of up to 60 V to a single set of components for a 16-unit smart sleeve array. Thus, in this present implementation three of these boards are used to supply power for stirring, heating, and LEDs (powered at 9V, 12V and 9V respectively).

Pump Control:

Switch arrays boards made by Sainsmart (sainsmart.com/relay-1/relays/sainsmart-imatic-16-channels-wifi-network-i-o-controller-kit-for-arduino-relay-android-ios.html) were used to control small inexpensive peristaltic pumps. This particular device implementation has 3 pumps per smart sleeve, for a total of 48 pumps in a single array. This allowed for devoting two pumps to input (one media and the other media+stress) and one to efflux. Switch arrays are controlled by an Arduino Mega connected to a 7.5 V DC power supply. Pump and smart sleeves are arranged as a tower, with the smart sleeve layer on top, and each of the three pump layers stacked below it.

Software:

All microcontrollers are connected to a generic PC laptop via USB connections. The system is controlled using custom code written in Python and Arduino C. The Python code consists of three long-running processes, responsible for data acquisition, data analysis, and pump driving. Data from either the DAQ device or the analog input circuit are processed and saved. Upon reaching the OD threshold, pump driving times are calculated and sent to the pump driving process. Data are sent through pipes, by connecting the output of one process to the input of another. Data is stored on the lab computer in the HDF5 format, a commonly-used file format for storing data in the scientific community.

The Arduino C code controls the stirring and temperature of the vials. Due to the high speed of the stirrers at even low voltages, pulse-width modulation is performed by in the Arduino software to achieve appropriate spin rates. Temperature control is performed by measuring the resistance of a thermistor (which changes with temperature) flush against the aluminum sleeve, and adjusting the current to the heating resistors using a proportional-integral-derivative controller written in Arduino C.

Data visualization software is written in HTML and JavaScript (JS), with data displayed using elements of from the Dygraphs (dygraphs.com) library. The HTML consists of a barebones website containing empty div Lags for JS to change dynamically. The JS code is responsible for setting up event handlers (e.g. menu clicks, zooms and drags), setting up Dygraph objects for displaying the data, and communicating with a Python Tornado server running on the lab computer over the WebSockets protocol for sending and retrieving data. This arrangement allows multiple users to view plotted data remotely by logging into a secure webpage onto which data being collected are dynamically streamed.

### Example 5. Methods for Examples 6-18

Strain Construction:

Genotypes for yeast and bacterial strains used in this study are listed in TABLE 2. Plasmids were constructed using standard molecular biology techniques. Strains were generated using standard lithium acetate transformation. When appropriate, non-isogenic pooled population samples were given unique designations for clarity (TABLE 2).

TABLE 2
Description of strains used in this study.

| Strain/Pool | Parental Strain | Description |
|---|---|---|
| FL100 | S. cerevisiae | MATa reference strain |
| | ATCC 28383 | |
| YJW509 | S. cerevisiae | MATα adel-14 his3-11,15 leu2-3 trp1-1 |
| | W303 strain | ura3-1 (Osherovich L.Z. and Weissman |
| | MATa haploid | J.S., Cell 106, 183-94 (2001)) |
| YJW564 | S. cerevisiae | MATa ade1-14 his3-11,15 leu2-3 trp1-1 |
| | W303 strain | ura3-1 (Osherovich L.Z. and Weissman |
| | MATα haploid | J.S., Cell 106, 183-94 (2001)) |
| TG1 | Escherichia | supE thi-1 Δ(lac-proAB) Δ(mcrB-hsdSM)5, |
| | coli K-12 | (rK-mK-) (Stratagene) |
| MG1655 | Escherichia | rph-1 |
| | coli K-12 | |
| | ATCC 47076 | |
| BS 303 | Serratia | type strain for Serratia marcescens |
| | marcescens | |
| | ATCC 13880 | |
| yBW001 | S. cerevisiae | adapted for 100 generations in eVOLVER, |
| | FL100 strain | founder for density dependent evolution |
| | | expt. |
| yBW002 | yBW001 | HO::pNH607 pTDH3-mNeonGreen |
| | | hygroR used in competitive fitness assay |
| | | for density dependent evolution expt. |
| ySK499 | cYJW509 | TRP1::pNH604 pGALI-mKate2 |
| | | used in glucose/galactose ratio sensing |
| | | expt. |
| ySK116 | cKJW584 | URA3::pRS306 pTEF1-mCherry |
| | | MATa founder for cyclohexamide |
| | | evolution vial in parallel evolution and |
| | | mating expt. |
| ySK743 | S. cerevisiae | LEU2::pNH605 pTET1-mNeonGreen |
| | W303 strain | MATα founder for ketoconazole evolution |
| | MATα haploid | vial in parallel evolution and mating expt. |
| | (from YJW509) | |
| yBW003 | ySK116/ | Diploid pool formed by mating ySK116/ |
| | ySK743 | ySK743 at to in parallel evolution and |
| | | mating expt. |
| yBW004 | +ySK116 | CHX-evolved MATa haploid pool |
| | | collected at t₁ in parallel evolution and |
| | | mating expt. |
| yBW005 | ySK116 | CHX-evolved MATa haploid pool |
| | | collected at t₂ in parallel evolution and |
| | | mating expt. |
| yBW006 | ySK743 | KETO-evolved MATα haploid pool |
| | | collected at t₁ in parallel evolution and |
| | | mating expt. |
| yBW007 | ySK743 | KETO-evolved MATα haploid pool |
| | | collected at t₂ in parallel evolution and |
| | | mating expt. |
| yBW008 | ySK116/ | Diploid pool formed by mating yBW004/ |
| | ySK743 | yBW006 at t₁ in parallel evolution and |
| | | mating expt. |
| yBW009 | ySK116/ | Diploid pool formed by mating yBW005/ |
| | ySK743 | yBW007 at t₂ in parallel evolution and |
| | | mating expt. |

Routine Cell Culture Techniques:

Culture conditions varied according to the needs of particular experiments. Cultures used to seed eVOLVER experiments were prepared as follows: Saccharomyces cerevisiae (obtained from frozen stock or single colonies) was grown in 2 mL of YPD media (2% glucose) at 30° C. in a shaking incubator (300 rpm) for at least 36 h. For routine overnight culture of Escherichia coli or Serratia marcescens, cells obtained from frozen stock were used to inoculate 2 mL of LB Miller broth grown at 37° C. for 12 h in a shaking incubator (300 rpm).

Flow Cytometry:

Flow cytometry was used to measure single-cell fluorescence throughout the study. Prior to measurement, 200 uL of yeast culture (see additional methods for experiment-specific growth conditions) was diluted with 100 uL of filter-sterilized PBS supplemented with cyclohexamide to a final concentration of 20 ug/mL, then incubated at 4° C. in the dark for no less than 3 h to allow for fluorophore maturation. An Attune NxT Flow Cytometer (Invitrogen) equipped with an autosampler was used to acquire data. For a typical experiment, at least 10.000 events were acquired. Cells were analyzed using FlowJo (Treestar Software). Intact cells were gated using forward and side scatter, followed by gating on fluorescence channels (green and/or red, as appropriate) to determine the fractional distribution of each population.

Fitness Calculations:

Competitive fitness, in which a strain of interest is co-cultured in competition with a reference strain, was assayed in the same fashion throughout the study. The ratio of the two strains was determined at multiple timepoints—generally at the beginning and end of an experiment—either by flow cytometry or qPCR. Fitness values, F, were calculated using the following equation (Kryazhimskiy S., et al., Science 344, 1519-1522 (2014)):

$$F = \frac{1}{{t}_{2} - {t}_{1}} log ( \frac{n ( {t}_{2} )}{{n}_{r} ( {t}_{2} )} / \frac{n ( {t}_{1} )}{{n}_{r} ( {t}_{1} )} )$$

where t is number of generations, and n and n, are cell counts for the strains of interest and reference strain, respectively.

Setup Procedure for eVOLVER Experiments:

Prior to each eVOLVER experiment, 40 mL borosilicate glass vials outfitted with a stir bar and capped with an influx port and an efflux port were sterilized by autoclave. Media and waste lines were sterilized by pumping 10% bleach (20 mL), followed by 70% ethanol (20 mL). Lines were cleared by pumping air for 20 s, followed by media (20 mL). Media and waste lines were then attached to the influx and efflux ports of each vial, and each vial filled by pumping 25 mL of the appropriate media through the influx port. At this point, Python control code was initiated, triggering a blank media measurement, and activating Smart sleeve heating elements. Prior to seeding with cells, fresh media was incubated in the device for a length of time sufficient for the first 15 optical density recordings to be taken (˜2.5 min) and for media to reach the programmed temperature.

Ertracting Yeast Genomic DNA:

To extract genomic DNA, ˜2×10⁶ yeast cells (roughly 30 μL of overnight culture) were pelleted by centrifugation (5 min, 1000 rcf). Supernatant was removed, and pellets were resuspended in 30 μL 0.2% sodium dodecyl sulfate (SDS), followed by vortexing for 15 s. Suspensions were transferred to PCR tubes and heated in a thermal cycler (37° C.) for 5 min, followed by 98° C. for 5 min before cooling to 4° C. Extracts were diluted with H₂O to a final volume of 75 p L prior to being used as PCR template. Primers used in this study are listed in TABLE 2.

Library Preparation and Barcode Sequencing:

Libraries for next-generation sequencing were prepared by PCR-amplification of genomic DNA (LightCycler 480 Instrument II, Roche), purification (Zymo Research). Primers used for indexing (culture, timepoint) and sequencing adapters were added via PCR. NextSeq sequencing (Harvard Biopolymers Facility) was used to sequence the culture index, the timepoint index, and a 55 bp single end read of the barcode construct. PhiX was added at 50% to increase sequence diversity. Alignment was performed using custom code harnessing MATLAB's Bioinformatics Toolbox and Boston University's parallel computing cluster. Alignment scores were calculated using the Smith-Waterman algorithm (swalign function) and assigned based on best score above a minimum threshold. In total, we assigned more than 244 million reads to 5149 unique library members to track frequency across the 4 time points for each of the 16 conditions.

Characterizing eVOLVER Operation and Stability with Long-Term Bacterial Growth:

To demonstrate a typical use case of eVOLVER and simultaneously characterize the operation and stability of the platform, we conducted a long-term E. coli growth experiment in turbidostat mode using a basic setup with peristaltic pumps for fluidic handling. Throughout the experiment, we monitored contamination, calibration settings, and stability of both software and hardware.

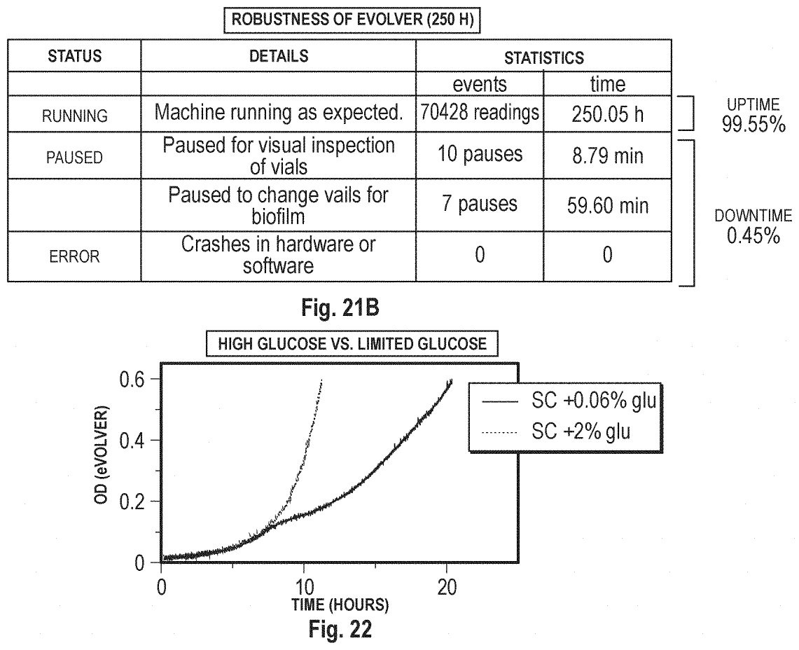

An overnight culture of E. coli MG1655 was grown in pre-buffered LB Miller media (pH 7.2). This culture was used to inoculate 25 mL of M9 media containing 0.4% glucose and 1 ug/L thiamine (hereafter referred to as M9), and this was grown to mid exponential phase. Meanwhile, 16 eVOLVER vials were initialized with antibiotic-free M9 media. Eight vials were inoculated with cells from the mid-exponential culture, interspersed with eight vials that were left uninoculated. The cultures, maintained at 37° C. in turbidostat mode (OD window=0.15-0.2), exhibited similar growth rates over the first 48 hours as expected (FIGS. 21A-21B).

To limit selection for biofilm, manual daily transfers were commenced after 72 hours of growth, when biofilm accumulation was initially observed. Sequentially, vials were removed from their Smart Sleeves, 2 mL of culture was diluted into a freshly autoclaved vial containing 23 mL fresh media, which were then inserted into the vacant Smart Sleeve. Transferring all eight cultures took approximately 10 minutes, and cultures did not appear to be negatively affected by these periodic transfers. The cultures were maintained for a total of 250 hours (1.5 weeks), constituting over 200 generations in exponential growth.

During this time, we observed no unplanned software or hardware failures, consistent with our other long-term culture experiments. Data collection was momentarily paused during routine vial examination and transfers, which constituted an expected downtime of less than 1% of experimental time (FIGS. 21A-21B). With continuous OD monitoring, we observed no changes in density in the uninoculated vials, suggesting that neither environmental contamination nor cross contamination occurred. Additionally, at the conclusion of the experiment, samples were plated on LB agar plates at two concentrations (undiluted and at 10⁶), and incubated for 24 hours at 37° C. Only E. coli colonies were isolated from plates derived from the eight active cultures, confirming that long-term continuous culture and vial transfers were indeed performed in a sterile manner. Moreover, no colonies were observed on plates derived from the un-inoculated vials, despite being located adjacent to actively passaged cultures and connected to the same media sources and waste as the active cultures.

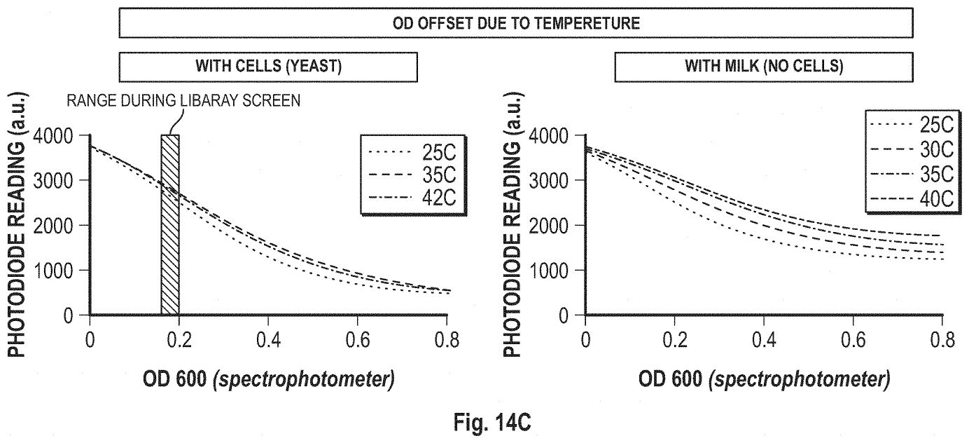

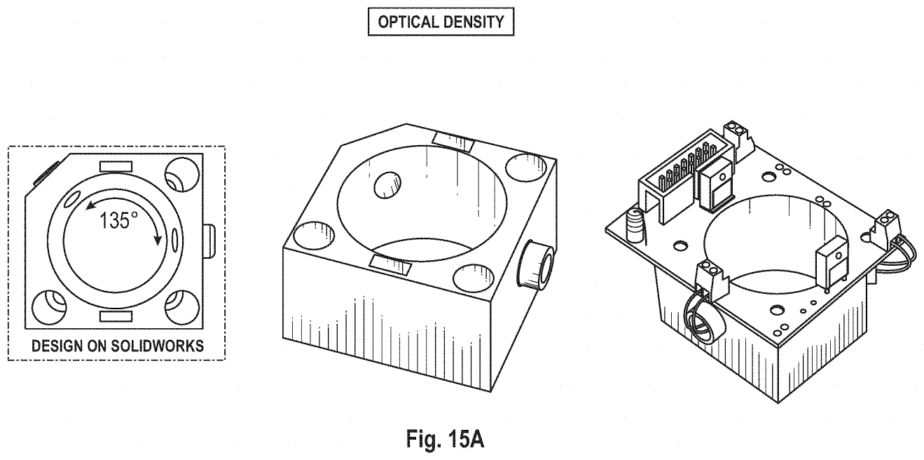

At the conclusion of the experiment, temperature, OD, and fluidics were all re-calibrated, and their calibration values compared to those from the start of the experiment (FIGS. 14A-14C, FIGS. 16A-16C, and FIGS. 18A-18B). Calibrations were found to be virtually unchanged over the weeks or months since initial calibration, spanning hundreds to thousands of hours of use, depending on the component. Taken together, this control experiment demonstrates that eVOLVER is capable of robust long-term operation, appropriate for experiments that would be typical across a wide variety of studies.

Conducting Experimental Evolution Across a Multidimensional Selection Space:

In order to showcase eVOLVER's ability to conduct long-term, continuous culture laboratory evolution experiments at high throughput, we explored the relationship between culture density and fitness in evolving yeast populations. To accomplish this, we configured the eVOLVER to function as a turbidostat, where culture density is continuously maintained within a constant, defined density window by an automated, OD-dependent media dilution routine. By varying the upper and lower thresholds that define each density window, we scanned a multi-dimensional space of population density at fine resolution to assess adaptive outcomes.

Parallel Evolution Across Density Space:

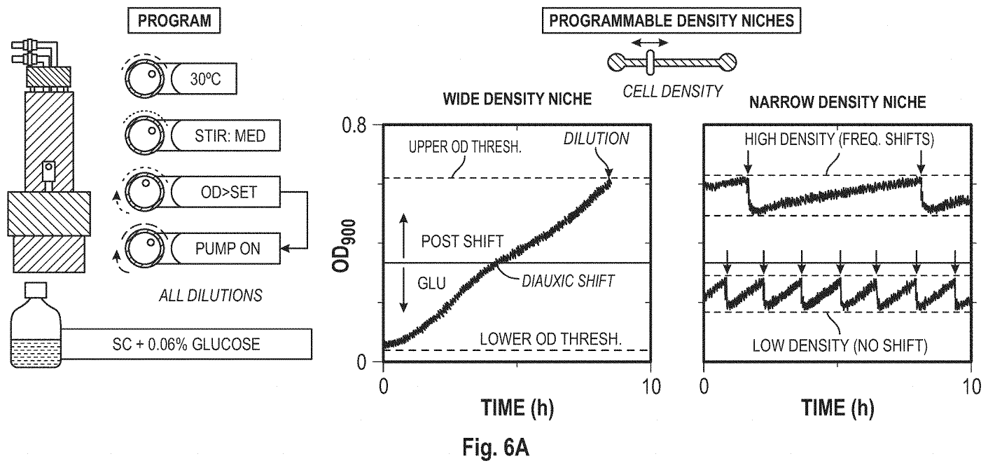

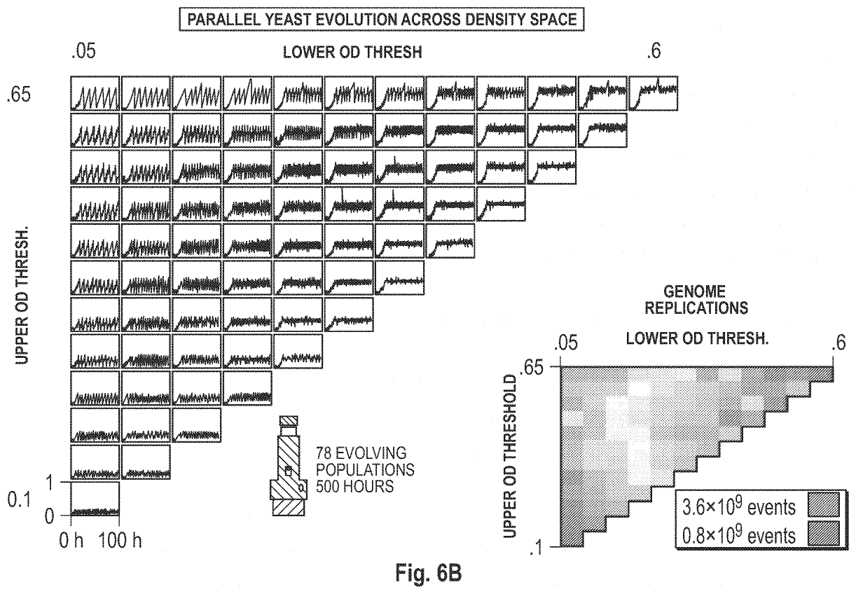

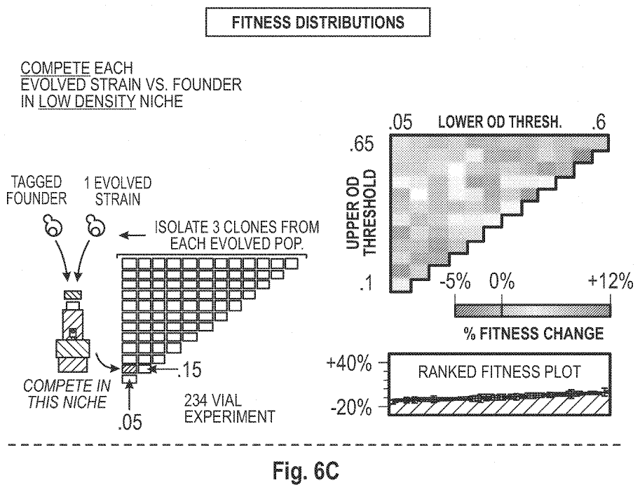

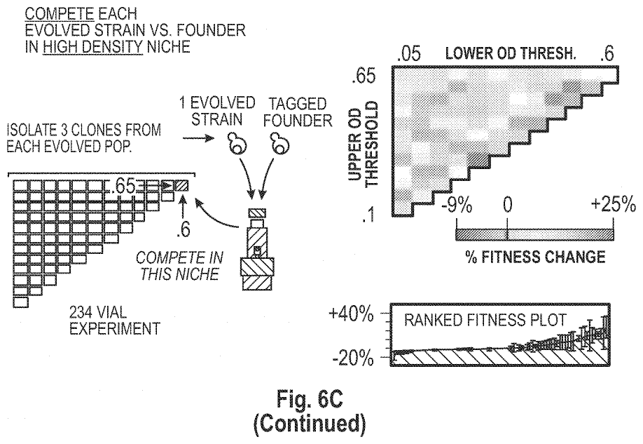

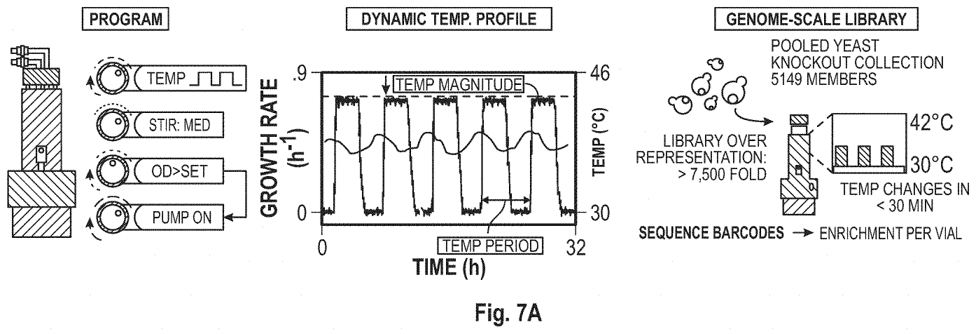

A single colony of prototrophic S. cerevisiae FL100 (ATCC 28383) was pre-adapted in eVOLVER continuous culture (turbidostat mode. OD 0.25-0.3) in Synthetic Complete (SC) medium+2% glucose (Sunrise Science Products) for 100 generations. A single colony from this population was selected as the founder strain (yBW001) for the high throughput, density-dependent evolution (FIGS. 6A-6C). An overnight culture of the founder was used to seed 78 parallel eVOLVER cultures at an initial OD 0.05. Each of the 78 populations was maintained in a specified density regime during continuous culture in SC+0.06% glucose+50 ug/mL carbenicillin+25 ug/mL chloramphenicol at 30° C. for 500 h (FIG. 6B). Density regimes were maintained using an automated feedback scheme between OD measurement and media dilution. Specifically, a dilution event is triggered by a culture reaching a specified upper OD threshold; the culture is then diluted to a specified lower OD threshold by activating the pumps for a duration time calculated by the software. Glucose-limited media was used to induce periodic diauxic shifts within the observable OD range (FIG. 22). Cultures were sampled every day. Frozen stocks were made by diluting 200 uL culture with 85 uL sterile 50% glycerol and stored at −80° C.

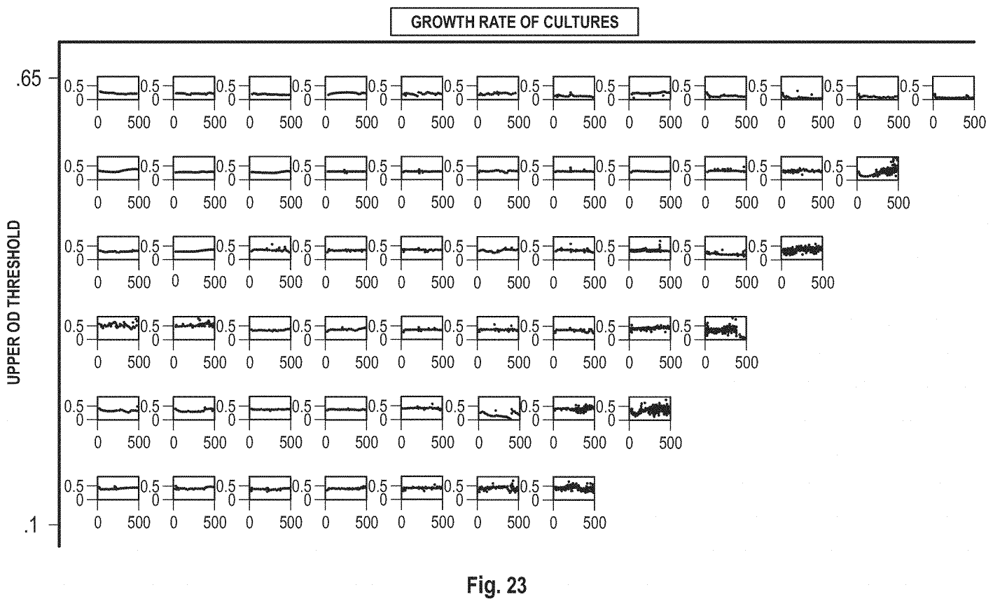

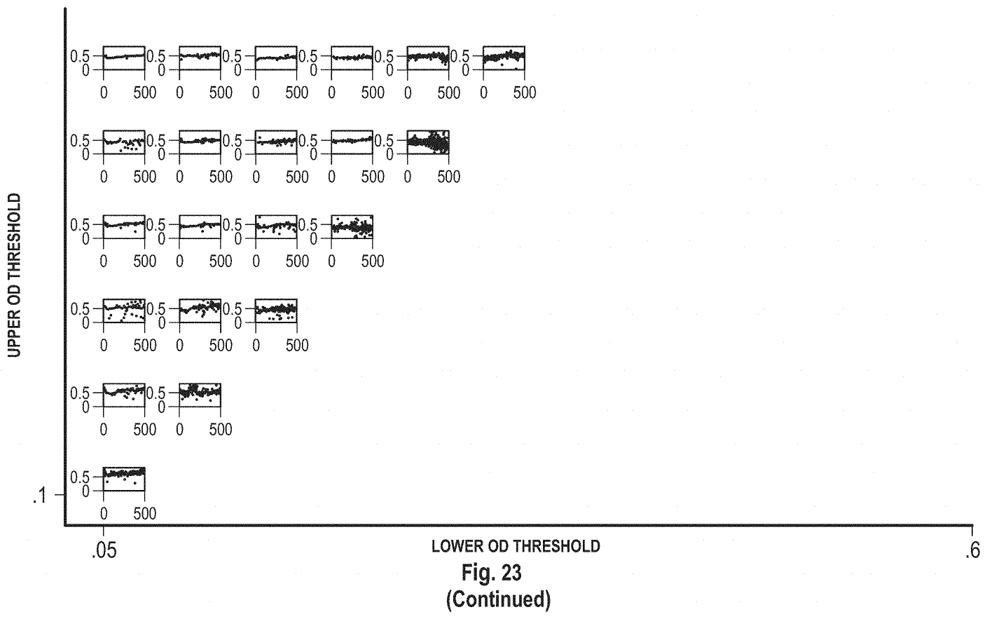

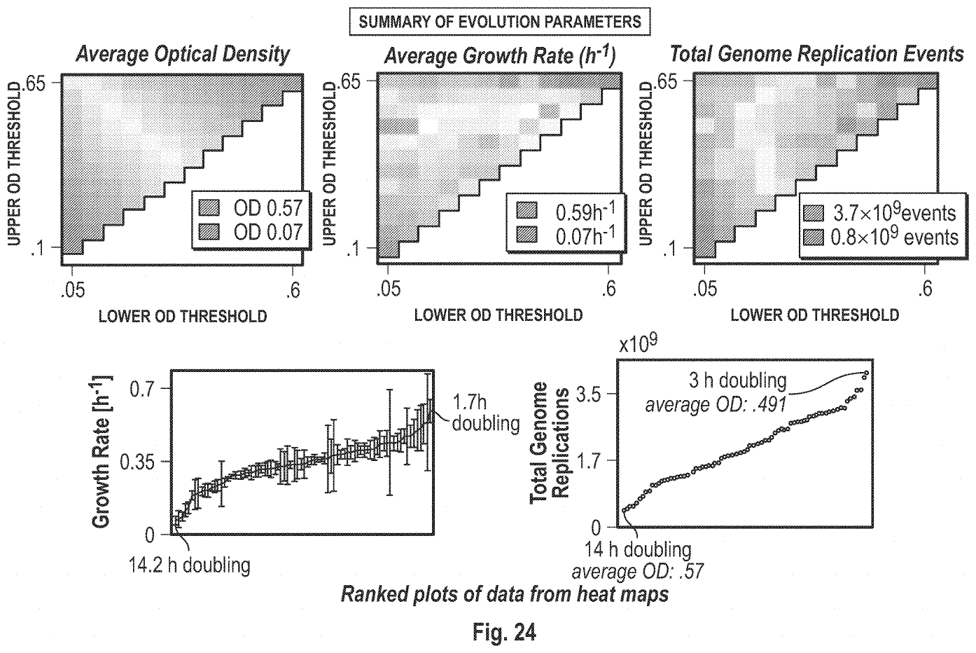

Culture densities were monitored continuously, permitting calculation of population size, mean doubling time (or growth rate), and genome replication events in each vial during the course of the experiment (FIG. 23 and FIG. 24). Notably, the regimes leading to the fastest growth (i.e. low density regimes corresponding to >200 cell generations) were not necessarily those associated with the greatest number of total genome replications, which is a function of both growth rate and population size (FIG. 6B and FIG. 24). It should be noted that these variable population sizes affect the degree to which different forces in population genetics, including genetic drift and mutational fixation, affect the outcome of evolution in each condition (Wahl L M., et al., Genetics 162, 961-971 (2002)).

At the limiting glucose concentration we used, cultures exhibit a reduced carrying capacity and observable metabolic or diauxic shifts. Consequently, by simply setting the upper and lower density thresholds of the culture with eVOLVER, we could observe an impact on the resulting metabolic niche. For example, if the density window is below the diauxic point, the characteristic shift is never observed; conversely, if the window is high, the population exhibits two distinct phases of growth. The duration in each phase and the number of shifts seen per generation of growth varies across the sampled landscape.

Competitive Fitness in Different Density Niches:

At the conclusion of 500 h of continuous growth, each culture was struck out on a YPD plate. Three colonies were picked from each plate, grown in SC+0.06% glucose, and frozen in glycerol. These stocks were used to seed competitive fitness assays against the fluorescently labeled founder strain (yBW002) (FIG. 6C). Specifically, for each pairwise competition, cells were grown in eVOLVER vials (SC+0.06% glucose+50 ug/mL carbenicillin+25 ug/mL chloramphenicol) to mid-exponential phase (OD ˜0.5), then diluted down to OD 0.05 using the peristaltic pump array. Meanwhile, a 200 mL culture of the founder (yBW002) was grown at 30° C. in a shaken flask to mid-exponential phase, then centrifuged and resuspended to OD 1.0 in fresh media. A 1 mL aliquot of yBW002 cell suspension was added to each eVOLVER vial to form a 1:1 ratio of founder to evolved strain at OD 0.1. Competition fitness experiments were performed in two density regimes: low density (OD 0.05-0.15) and high density (OD 0.6-0.65) (FIG. 6C). Cells were sampled for flow cytometry at two time points: t=0 generations (after reaching the desired density regime) and t=8-10 generations (length of experiment set by depletion of media). Quantification of fluorescence ratios was used to calculate relative fitness.

Characterization of Evolutionary Parameters on Niche Fitness:

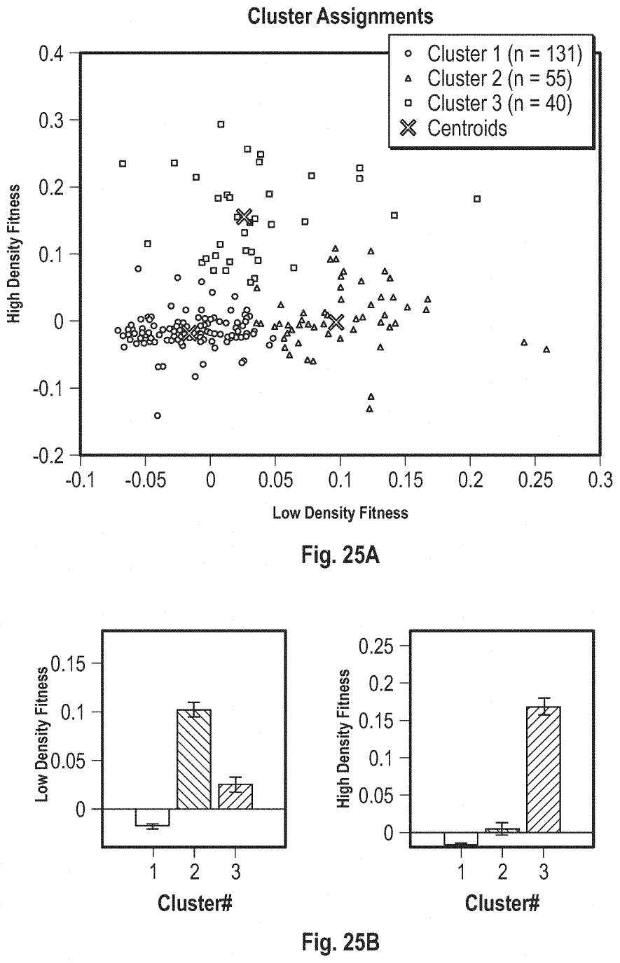

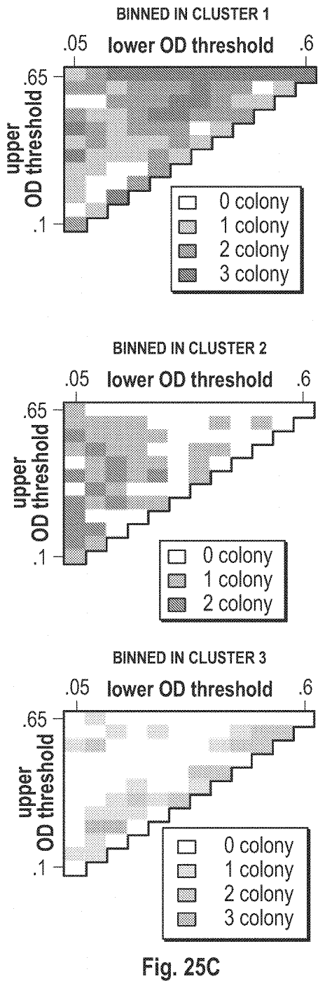

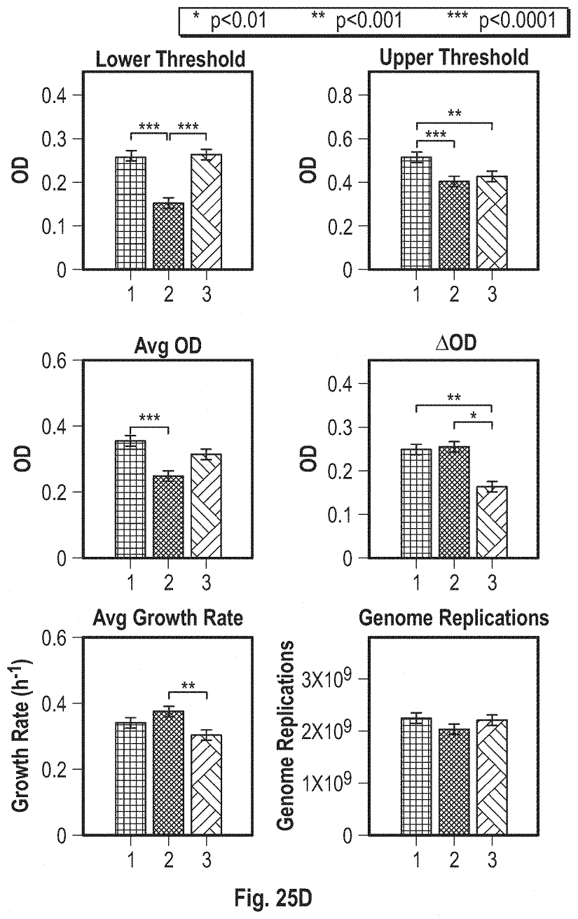

Next, we wanted to determine if resulting fitness measurements were significantly correlated with any unique environmental parameter during evolution. High and low density fitness measurements for each colony were plotted on the same scatter plot and clustered using k-means, yielding three distinct groups: low-density specialists, high-density specialists, and colonies with low fitness in both measured niches (FIGS. 25A-25D). Clustering with more than three groups resulted in subdivisions of one of the above three clusters and did not exhibit significant fitness differences. We then generated heatmaps describing how individual colonies in each cluster mapped back to the original evolutionary niche (FIGS. 25A-25D). To quantitatively describe how evolutionary history correlated with fitness measurements, we performed student's t-tests comparing imposed conditions and recorded traits of each cluster (FIGS. 25A-25D). This analysis revealed lower OD threshold, Δ OD, and upper OD threshold to be significant distinguishing features for low-density, high-density, and low-fitness clusters, respectively. For example, low-density specialists were derived from density windows with a significantly smaller lower threshold. These results reveal how simple modifications to evolutionary niches result in diverse and non-intuitive selection pressures. For example, the high-density specialist cluster is differentiated by narrow density windows (Δ OD) rather than a high average OD or upper OD threshold, as one might have expected. With eVOLVER, we have the ability to continuously measure, record, and analyze the evolutionary history of the culture to correlate these parameters to resulting changes in fitness.

Growth Selection Under Temporally Varying Selection Regimes:

There is growing interest in interrogating biological systems in fluctuating conditions that more closely reflect the dynamics of natural environments (Berry D. B., et al., PLoS Genet. 7, e1002353 (2011); Gibney P. A., et al., Proc. Natl. Acad. Sci. 110, E4393-E4402 (2013)). In fluctuating environments, different phenotypes and adaptations may arise than in environments with monotonic selection pressure. eVOLVER makes it possible systematically study the relationship between temporal fluctuation and phenotypic selection, while holding other environmental variables constant. To demonstrate this, we performed growth selection experiments on a pooled yeast knockout library (Giaever G., et al., Nature 418, 387-391 (2002); Breslow D. K., et al., Nat. Methods 5, 711-718 (2008); Li Z., et al., Nat. Biotechnol. 29, 361-7 (2011); Giaever G. and Nislow C., Genetics 197, 451-465 (2014)), under conditions in which a single environmental variable—temperature—was temporally varied.

Dynamic Temperature Selection on Yeast Knockout Library:

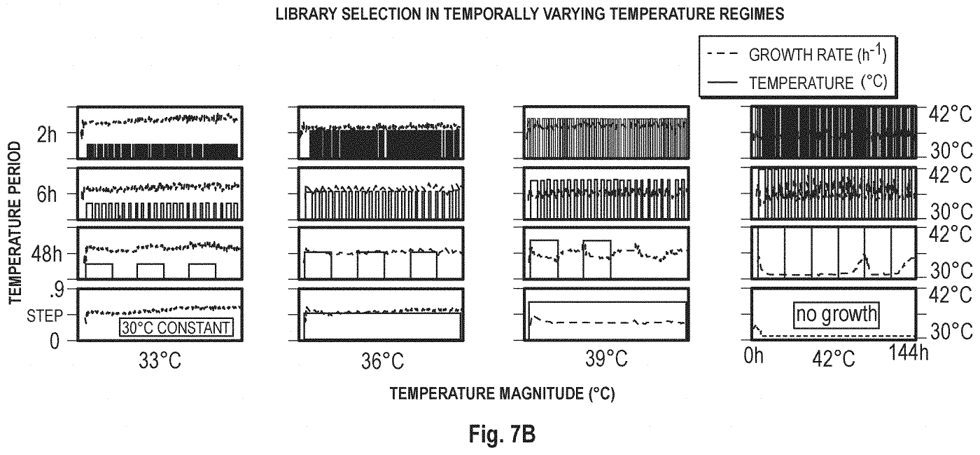

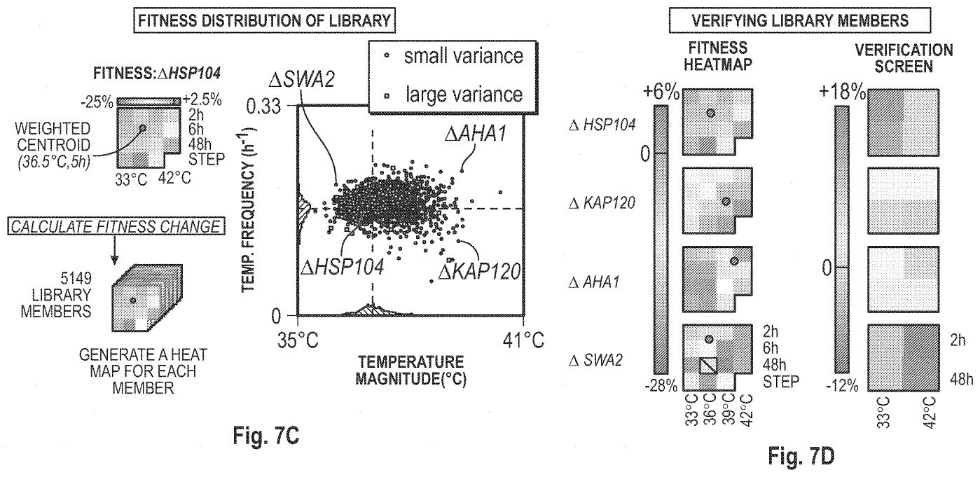

A 500 uL aliquot of the pooled haploid MATα yeast knockout collection (Transomic TKY3502P) was thawed and grown in 500 mL YPD under constant shaking (300 rpm) at 30° C. for 12 h. Cells were then seeded in eVOLVER vials (containing YPD+50 ug/mL carbenicillin+25 ug/mL chloramphenicol) at an initial OD 0.05. Cultures were grown at 30° C. for 5.5 h in order to reach OD ˜0.15, and then maintained in continuous turbidostat culture (OD 0.15-0.20) over the course of the experiment. Temperature perturbations of varying magnitude and period (FIGS. 7A-7B) were initiated as soon as regular dilution events were underway in all cultures. Temperature magnitude and period were selected based on thermal range calibrations performed prior to the experiment (FIGS. 14A-14C).

Growth rate was clearly observed to vary in response to temperature changes (FIG. 7B). At low and intermediate temperature magnitudes, growth rate appears to increase along with temperature. At higher temperatures, growth rates drop significantly during periods of thermal stress. Notably, no bulk growth was observed for cultures in the 42° C./48 hr or 42° C./step during periods of heat stress. During these periods, no dilution events occur, and therefore the samples may not be enriched for resistant members of the library as less fit members fail to be removed from the culture. However, the growth rate does recover during periods of permissive temperature in the 42° C./48 hr population. The onset of this recovery is hastened over time, suggesting that the cultures may be enriched during the periods of recovery at permissive temperature as cells that survive the elevated temperature reproduce.

Two mL culture samples were taken every 48 h for six days and frozen at −80° C. For actively growing cultures, regular automated dilution events were sufficient to replace culture volume lost by sampling. For the 42° C./step condition, in which no dilution events were triggered because of a lack of growth, fresh YPD was manually added to replace culture volume after each sample. Genomic DNA was extracted from each of the 64 samples (16 temperature profiles×4 timepoints) by thawing at room temperature, pelleting cells at 1000 rcf for 5 min, and then performing a genomic extraction protocol.

Library Preparation and Barcode Sequencing:

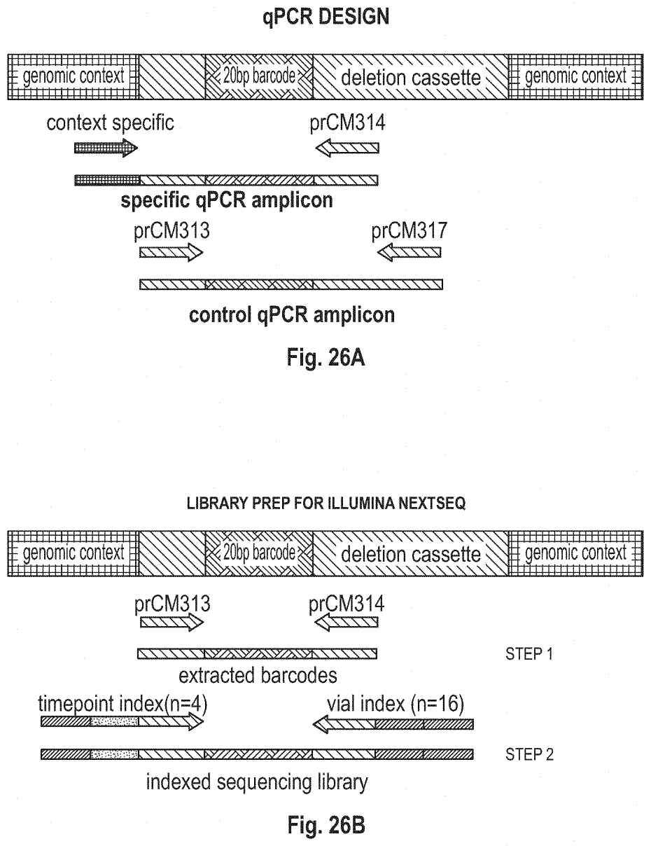

Library preparation was performed in two stages (FIG. 26B), normalizing DNA concentration between stages in order to minimize amplification bias from saturation behavior in PCR. In the first stage, barcodes were extracted from genomic DNA. First, a 1 uL aliquot of genomic DNA template from each sample was amplified in a LightCycler 480 Instrument II (Roche) with SYBR Green I Master Mix (Roche) using primers prCM313 and prCM314 (TABLE 3) and the following cycle conditions: (i) denaturation: 95° C. for 10 min; (ii) amplification (35 cycles): 95° C. for 10 s, 63° C. for 5 s. 72° C. for 14 s; (iii) elongation: 72° C. for 7 min. The resulting qPCR data was used to quantify the amount of target DNA present in each sample; this measurement was then used to normalize the DNA concentration across each of the 64 samples and determine a non-saturating number of cycles. Two uL of normalized sample DNA was then amplified with Q5 polymerase (New England Biolabs) using primers prCM361 and prCM362 (TABLE 3) in a 50 uL reaction using the following cycle conditions: (i) denaturation: 95° C. for 10 min; (ii) extension (5 cycles): 95° C. for 10 s, 64° C. for 10 s, 72° C. for 14 s; (iii) amplification (20 cycles): 95° C. for 10 s, 72° C. for 20 s; (iv) elongation: 72° C. for 7 min. Resulting DNA was purified using a DNA Clean Concentrator Kit (Zymo Research). To normalize samples again prior to the second round, DNA samples were quantified via qPCR using the same primers and conditions as before, then diluted to equal concentration.

In the second stage, indexes and sequencing adapters were added for every timepoint-vial combination, using a small number of cycles to minimize amplification. Amplification with i5-indexed primers prCM363-366 paired with i7-indexed primers prCM373-388 (TABLE 3) was performed in a 50 uL reaction using the following cycle conditions: (i) denaturation: 95° C. for 10 min; (ii) extension (5 cycles): 95° C. for 10 s, 65° C. for 10 s, 72° C. for 20 s; (iii) amplification (7 cycles): 95° C. for 10 s, 72° C. for 20 s; (iv) elongation: 72° C. for 7 min. Resulting DNA was again purified using a DNA Clean Concentrator Kit. DNA concentrations were determined using a Nanodrop Onec Spectrophotometer, and were mixed in equimolar amounts to form the final indexed library pool. The pool was diluted to 1 ng/uL and submitted to the Biopolymers Facility (Harvard Medical School). NextSeq sequencing was used to sequence the 8 bp i5 index, the 8 bp i7 index, and a 55 bp single end read of the barcode construct. Due to shared sequences in the regions flanking the barcode. PhiX was spiked in at 50% to increase sequencing diversity.

TABLE 3
Primers generated for this study.

| Primer No. | Name/Description | Sequence |
|---|---|---|
| prCM313 | YKO up f | GATGTCCACGAGGTCTCT |
| prCM314 | YKO up r | GTCGACCTGCAGCGTAC |
| prCM317 | YKO kanMX r | CTGCAGCGAGGAGCCGTAAT |
| prCM331 | HSP104 f | GAAATCAACTACACGTACCATAAAATATACAG |
| prCM338 | KAP120 f | CAACTGTCAACCGAATCAAATTTTAAAAG |
| prCM339 | AHA1 f | GTCTTATTCTTAATCGTTTATAGTAGCAACAAT |
| | | ATATC |
| prCM343 | SWA2 f | TCGTGGACTAGAGCAAGATTTC |
| prCM345 | HO f | CATATCCTCATAAGCAGCAATCAATTC |
| prCM361 | uptag1 | ACACTCTTTCCCTACACGACGCTCTTCCGATCT |
| | seq round 1 f | GATGTCCACGAGGTCTCT |
| prCM362 | uptag2 | GACTGGAGTTCAGACGTGTGCTCTTCCGATCTG |
| | seq round 1 r | TCGACCTGCAGCGTAC |
| prCM363 | i5001 | AATGATACGGCGACCACCGAGATCTACACAAC |
| | Day 0 | TCGCTACACTCTTTCCCTACACGAC |
| prCM364 | i5002 | AATGATACGGCGACCACCGAGATCTACACTTG |
| | Day 2 | AGCGAACACTCTTTCCCTACACGAC |
| prCM365 | i5003 | AATGATACGGCGACCACCGAGATCTACACAAG |
| | Day 4 | CCATTACACTCTTTCCCTACACGAC |
| prCM366 | i5004 | AATGATACGGCGACCACCGAGATCTACACTTC |
| | Day 6 | GGTAAACACTCTTTCCCTACACGAC |
| prCM373 | i7001 | CAAGCAGAAGACGGCATACGAGATTAAGGCGA |
| | 30° C./step | GTGACTGGAGTTCAGACGTGT |
| prCM374 | i7002 | CAAGCAGAAGACGGCATACGAGATCGTACTAG |
| | 33° C./2 h | GTGACTGGAGTTCAGACGTGT |
| prCM375 | i7003 | CAAGCAGAAGACGGCATACGAGATAGGCAGA |
| | 33° C./6 h | AGTGACTGGAGTTCAGACGTGT |
| prCM376 | i7004 | CAAGGAGAAGACGGCATACGAGATTCCTGAGC |
| | 33° C./48 h | GTGACTGGAGTTCAGACGTGT |
| prCM377 | i7005 | CAAGCAGAAGACGGCATACGAGATGGACTCCT |
| | 36° C./step | GTGACTGGAGTTCAGACGTGT |
| prCM378 | i7006 | CAAGCAGAAGACGGCATACGAGATTAGGCATG |
| | 36° C./2 h | GTGACTGGAGTTCAGACGTGT |
| prCM379 | i7007 | CAAGCAGAAGACGGCATACGAGATCTCTCTAC |
| | 36° C./6 h | GTGACTGGAGTTCAGACGTGT |
| prCM380 | i7008 | CAAGCAGAAGACGGCATACGAGATCGAGGCTG |
| | 36° C./48 h | GTGACTGGAGTTCAGACGTGT |
| prCM381 | i7009 | CAAGCAGAAGACGGCATACGAGATAAGAGGC |
| | 39° C./step | AGTGACTGGAGTTCAGACGTGT |
| prCM382 | i7010 | CAAGCAGAAGACGGCATACGAGATGTAGAGG |
| | 39° C./2 h | AGTGACTGGAGTTCAGACGTGT |
| prCM383 | i7011 | CAAGCAGAAGACGGCATACGAGATGCTCATGA |
| | 39° C./6 h | GTGACTGGAGTTCAGACGTGT |
| prCM384 | i7012 | CAAGCAGAAGACGGCATACGAGATATCTCAGG |
| | 39° C./48 h | GTGACTGGAGTTCAGACGTGT |
| prCM385 | i7013 | CAAGCAGAAGACGGCATACGAGATACTCGCTA |
| | 42° C./step | GTGACTGGAGTTCAGACGTGT |
| prCM386 | i7014 | CAAGCAGAAGACGGCATACGAGATGGAGCTAC |
| | 42° C./2 h | GTGACTGGAGTTCAGACGTGT |
| prCM387 | i7015 | CAAGCAGAAGACGGCATACGAGATGCGTAGTA |
| | 42° C./6 h | GTGACTGGAGTTCAGACGTGT |
| prCM388 | i7016 | CAAGCAGAAGACGGCATACGAGATCGGAGCCT |
| | 42° C./48 h | GTGACTGGAGTTCAGACGTGT |
| prCM353 | ERG3 f1 | TGAAGTGGTTGCAGAGG |
| prCM354 | ERG3 r1 | CCACTTGTGATGAGGCTTG |
| prCM355 | ERG3 f2 | GGAAGCTCATTATCGAGTACTTC |
| prCM356 | ERG3 r2 | CGAATAGCGCATATTGCAC |
| prCM357 | RPL41A f | GACTGTACTTTTCTGATGCG |
| prCM358 | RPL41A r | CTACATTGGGTATCACTCAAGTC |
| prCM359 | RPL41B f | CTGCGATGCTATCCATITAC |
| prCM360 | RPL41B r | CGGTAACAGCATCTTGCATAG |

Sequencing Alignment and Frequency Computations:

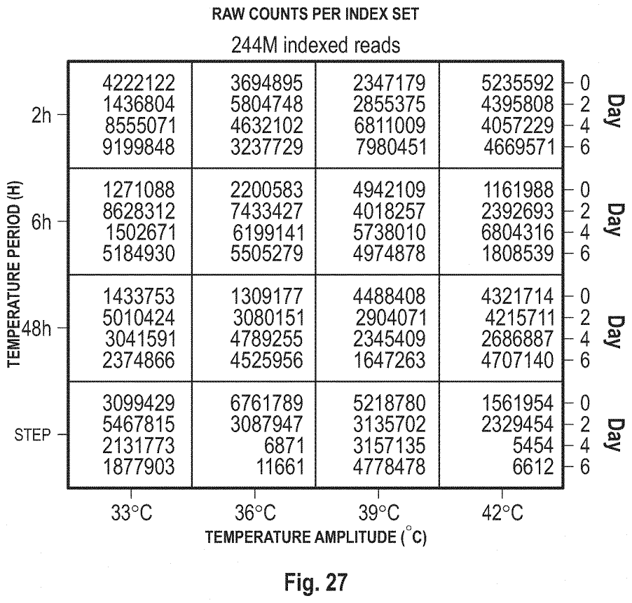

Alignment was performed using custom code harnessing MATLAB's Bioinformatics Toolbox and Boston University's parallel computing cluster. Reads were tabulated for each vial and timepoint using the index sequences (FIG. 27)), and assigned to the nearest barcode sequence indicated on the yeast knockout collection database (Giaever G. and Nislow C., Genetics 197, 451-465 (2014)). Alignment scores were calculated using the Smith-Waterman algorithm (swalign function) and assigned based on best score above a minimum threshold. Four samples had a significantly lower number of reads than the library mean, suggesting that the library pool was not comprised of equimolar samples; these timepoints and samples were excluded for principle component analysis and fitness centroid calculations, as noted below.

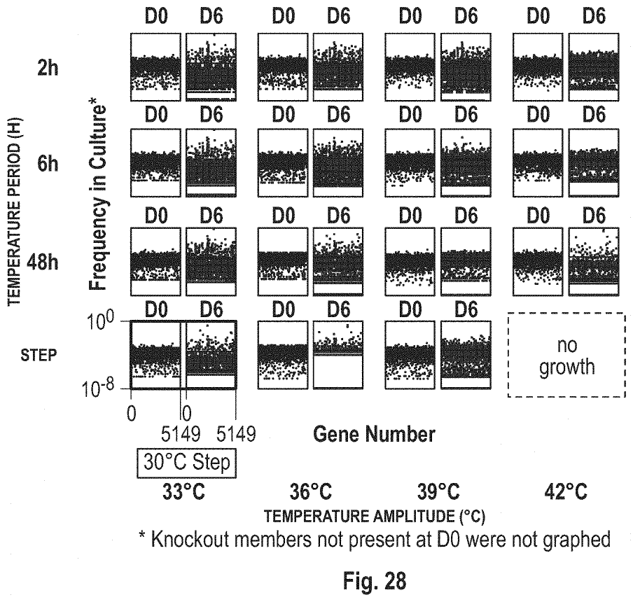

Population frequency of each library member was calculated by dividing the number of reads assigned to each member by the total number of assigned reads for a given indexed sample (FIG. 28). Wider frequency distributions were observed at Day 6 (compared to Day 0), as a few members increased in frequency, while many members decreased in frequency, often by orders of magnitude, indicating specific enrichment for each condition. Similarities in the enrichment pattern may also suggest similarities between the conditions themselves.

It should also be noted that while the sequencing depth is sufficient for the evenly distributed samples at Day 0, the wide range of frequencies observed following enrichment would be better measured at higher sequencing depth with reduced multiplexing of samples. As a result, we were careful not to draw strong conclusions from library members present at low frequencies in downstream analysis.

Cross-Correlation Between Temperature Conditions:

By prescribing experimental parameters in a programmable fashion, eVOLVER allows us to scan through environmental spaces. This allows us to identify similarities and differences in how these environments affect fitness outcomes, drawing conclusions about the environments themselves. For this experiment, we used the library performance data in order to delineate regions in temperature magnitude-frequency space that exert similar selection pressures on cells.

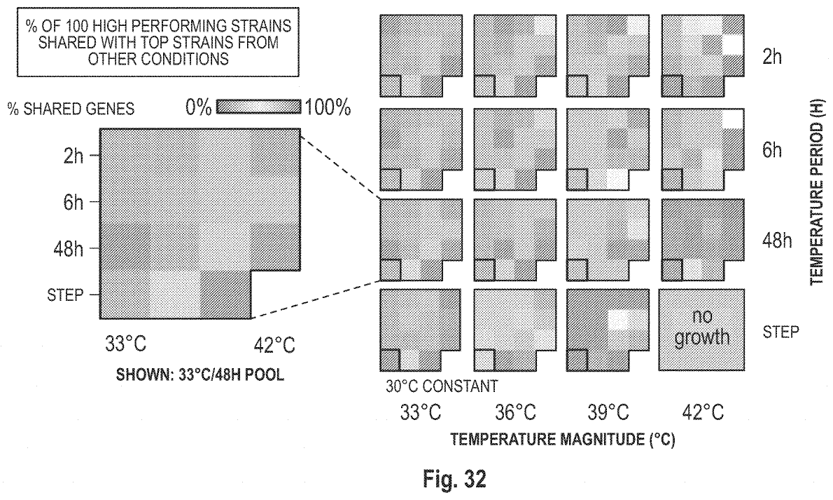

As a first pass, we looked at the correlation in 100 high-performing members from each condition. High-performing members were defined as those with the largest arithmetic difference in frequency between initial and final timepoints. When the overlap between these high-performing strains is quantified by tabulating the number of members shared between each condition (a simplified cross-correlation metric), relationships in the temperature magnitude-frequency space are revealed (FIG. 32).

When comparing the degree of similarity between a particular condition and each other condition, two regions of interest emerge. One region comprised of the conditions of low/moderate temperature magnitude and moderate/high temperature frequency exhibits a large degree of overlap, suggesting these conditions exert similar selective pressures on cells. In contrast, the conditions on the periphery, particularly those with high temperature magnitude, exhibit minimal overlap with other conditions, suggesting that unique selection pressures are at work. This approach is limiting however, as the definition of “high-performing members” biases which members are considered, rather than considering the entire dataset.

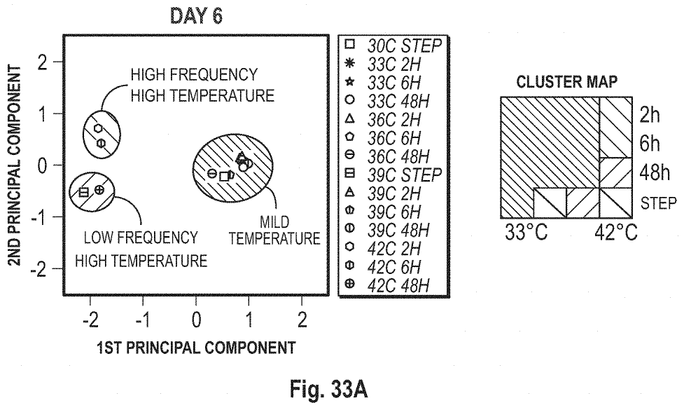

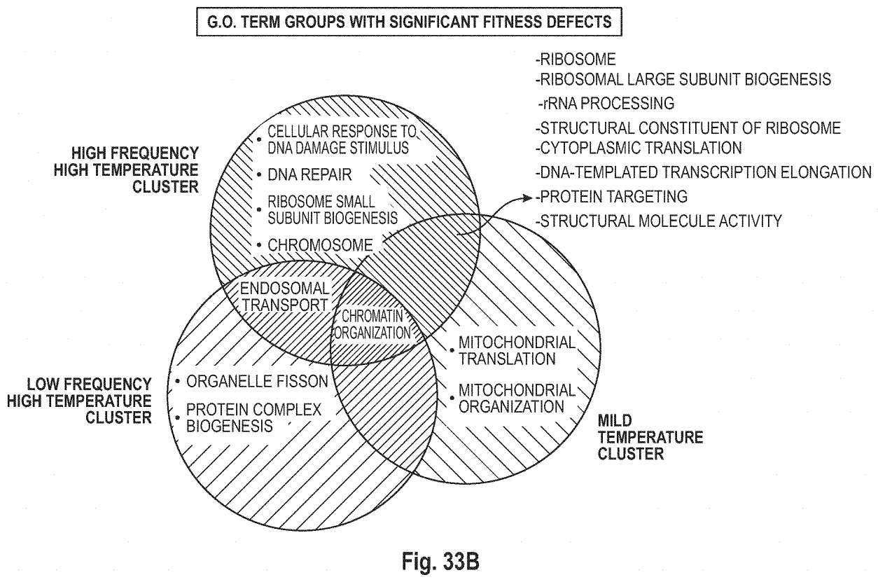

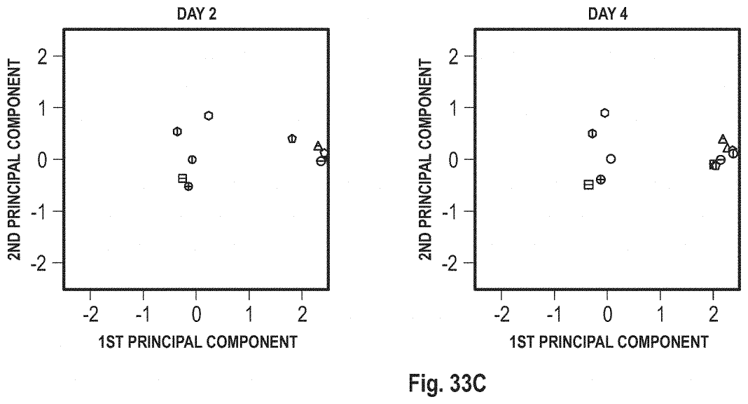

To further examine the similarities and differences between conditions, principle component analysis was applied. First, the arithmetic difference in frequency between the initial and final timepoints was calculated for each library member, tabulating the results in a vector for each condition. Library members that were missing at the Day 0 timepoint were excluded from analysis. The data from the 36° C./step and 42° C./step conditions were excluded from further analysis due to insufficient read depth. The frequency difference vectors were used to construct a 14×14 cross-correlation matrix to quantify the similarity between conditions. Principle component analysis was applied to the resulting cross-correlation matrix to separate the conditions across two axes (FIG. 33A) indicating the degree to which different conditions affect deletion mutant frequency in similar ways. We processed the data from the earlier timepoints similarly, then projected the cross-correlation results onto the same principle components calculated from the Day 6 data (FIG. 33C).

We observed three clusters in PCA coordinate space that are relatively stable over time: one large group that clusters with the mild temperatures, and two small clusters, corresponding to high temperature/high frequency, and high temperature/low frequency conditions. The library was divided into subsets with shared Saccharomyces Genome Database (Cherry J. M., et al., Nucleic Acids Res. 40, D700-D705 (2012)) (SGD) annotations of gene ontology (GO) Welch's t-statistic was applied to determine whether these GO terms are linked to significant changes in fitness for the conditions which comprise each PCA cluster. Correcting for multiple hypotheses, we found several cellular functions to significantly affect fitness in one or more of these PCA clusters (FIG. 33B). As expected, we observed that functions directly tied to growth rate (e.g. mitochondrial function, ribosome biogenesis) significantly altered fitness at mild temperature increases. Interestingly, ribosome components and processing factors also showed high-frequency sensitivity at high temperatures, suggesting a potential role for ribosome biogenesis in transitions in and out of stress. We further interrogated potential sources of frequency-dependence. We found that the high- and low-frequency groups were characterized by annotations associated with cell cycle checkpoints (e.g. DNA damage response, organelle fission), which temporally regulate cellular processes and thus might be expected to affect cellular response to fluctuating stresses at different frequencies.

Fitness Centroid Calculations:

Using eVOLVER to scan along different experimental parameters results in multidimensional fitness data spanning an environmental space, which can be challenging to visualize and interpret. In order to aid in analysis and visualization, we chose to transform the fitness of library members into temperature magnitude-frequency space. To do this, we computed a weighted fitness centroid, compiling the fitness in each condition into a pair of coordinates in temperature magnitude-frequency space.

Mean fitness of each library member in a particular condition can be calculated over different time periods using the population frequency in place of a ratio between two strains. Here the fitness computed over the Day 0-Day 6 range was used for all downstream analysis. Fitness centroids for each library member were calculated by averaging the coordinates of each condition in temperature magnitude-frequency space, with the fitness in each condition serving as weights. In this manner, library members with differential performance across conditions would exhibit shifted fitness centroids towards conditions in temperature magnitude-frequency space in which they were more fit (FIG. 7C). In order to avoid quantitative bias due to low sequencing depth, fitness calculations based on initial population frequencies below 10⁻⁵ were excluded from the centroid calculation. If more than three conditions of the heat map were excluded in this manner, a fitness centroid was not calculated for that library member.

To visualize the dataset of fitness centroid calculations, the centroid from each library member was plotted in a single scatter plot along the axes of temperature magnitude and temperature frequency (FIG. 7C). The mean centroid for the population is shifted slightly towards lower temperature magnitude and higher temperature frequency (or conversely, away from higher temperature and smaller frequency).

The fitness centroid approach has both advantages and disadvantages. The fitness centroid metric allows us to capture the relationships between the multidimensional parameters that prescribe each condition. The metric has proved very useful for simplifying and visualizing the complex data that results from experiments, which seek to map a parameter space; similarly, this type of data compression may prove useful for quantitative comparison between strains and groups of strains. However, as centroids are a non-monotonic metric, this compression also results in a loss of information. Consider two strains: Strain A is more fit at low temperature but equivalent to the reference strain at high temperature; Strain B is equally fit to the reference at low temperatures, but exhibits a fitness deficit at high temperature. In the fitness centroid metric, both strains exhibit a preference for low temperatures, and would therefore overlap. In another pathological example, any strain with a symmetric fitness profile with respect to a two-dimensional parameter space would have the same centroid, regardless of whether fitness is at a minimum, maximum, or uniform at that point. This may of course be addressed by reporting additional metrics, such as mean fitness, or higher-order derivatives of the landscape. Nevertheless, particularly for the fitness landscape being examined in this experiment, the fitness centroid metric has proved to be a valuable analysis tool.

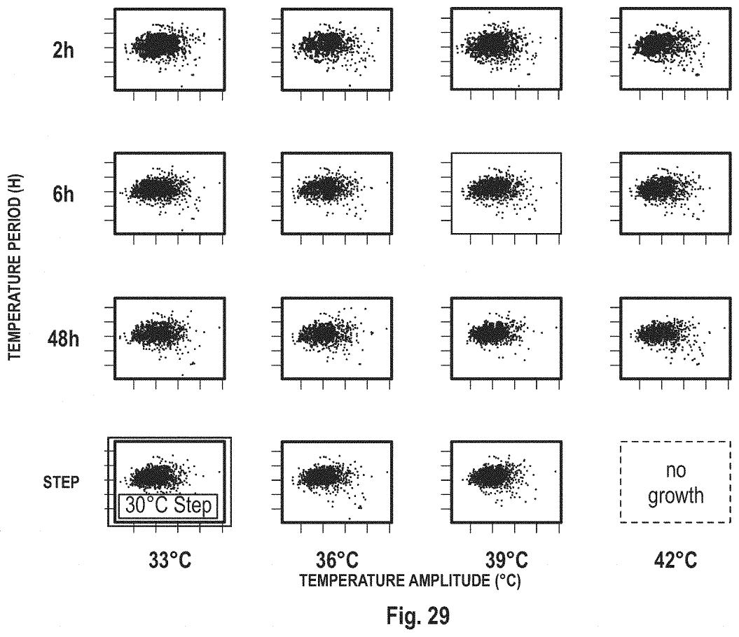

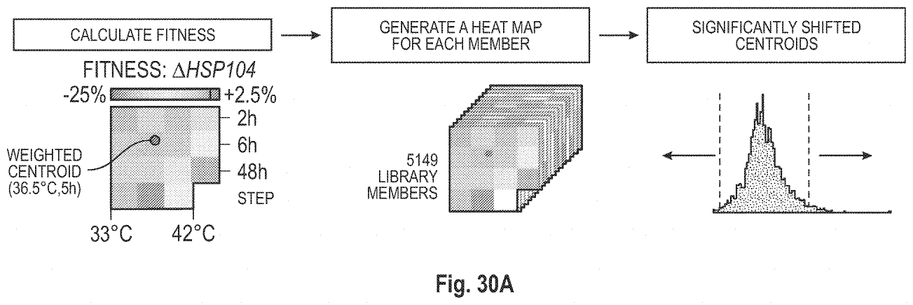

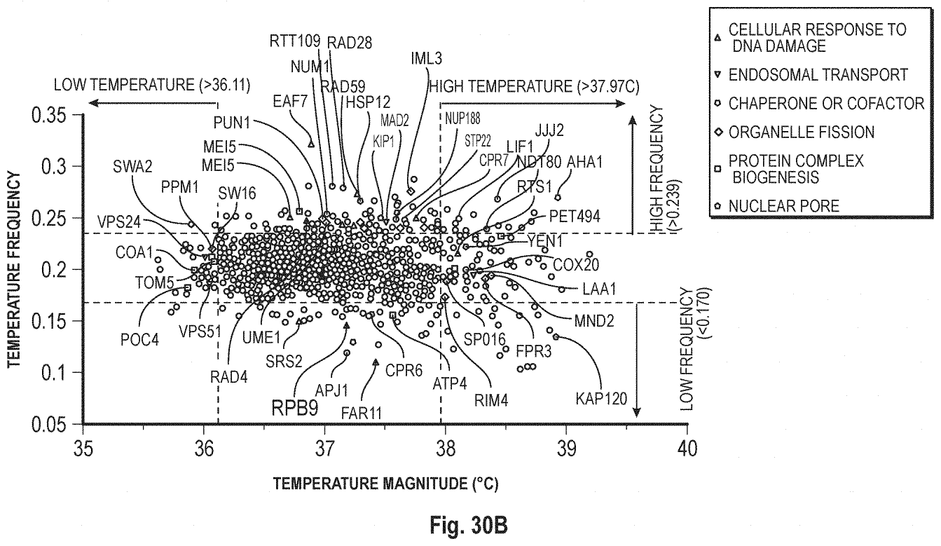

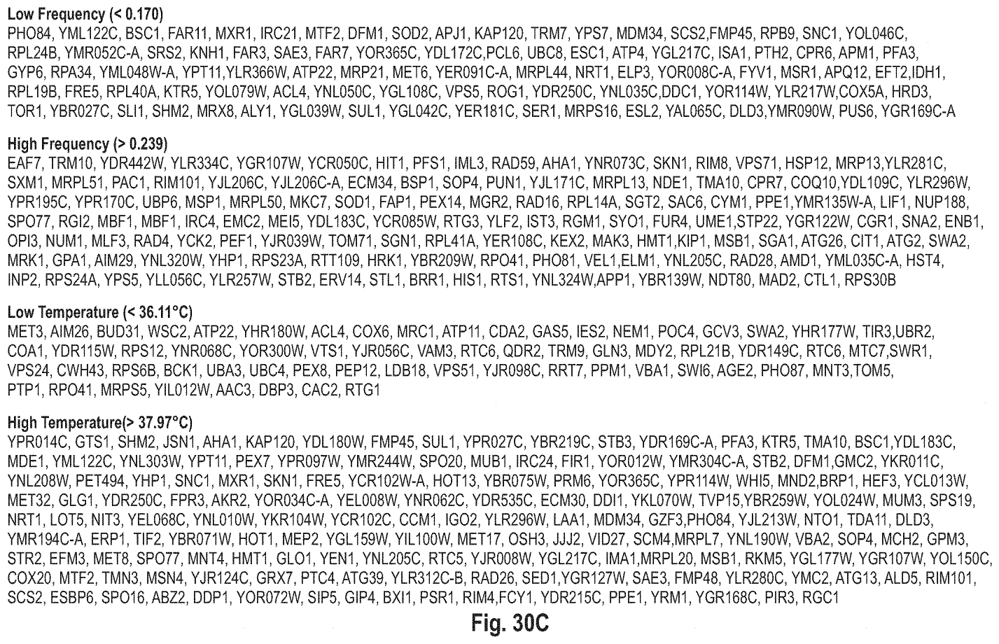

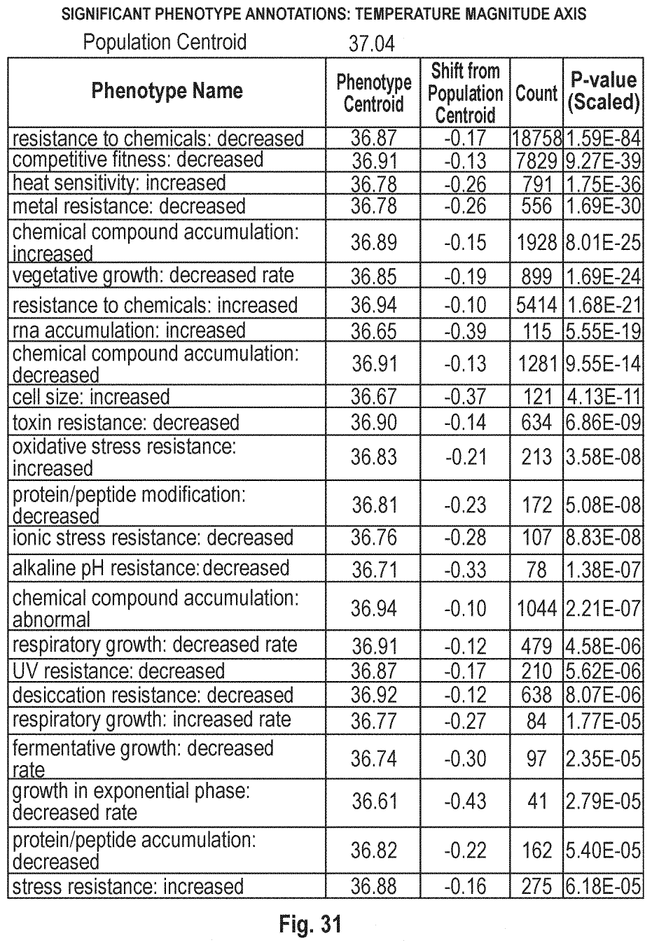

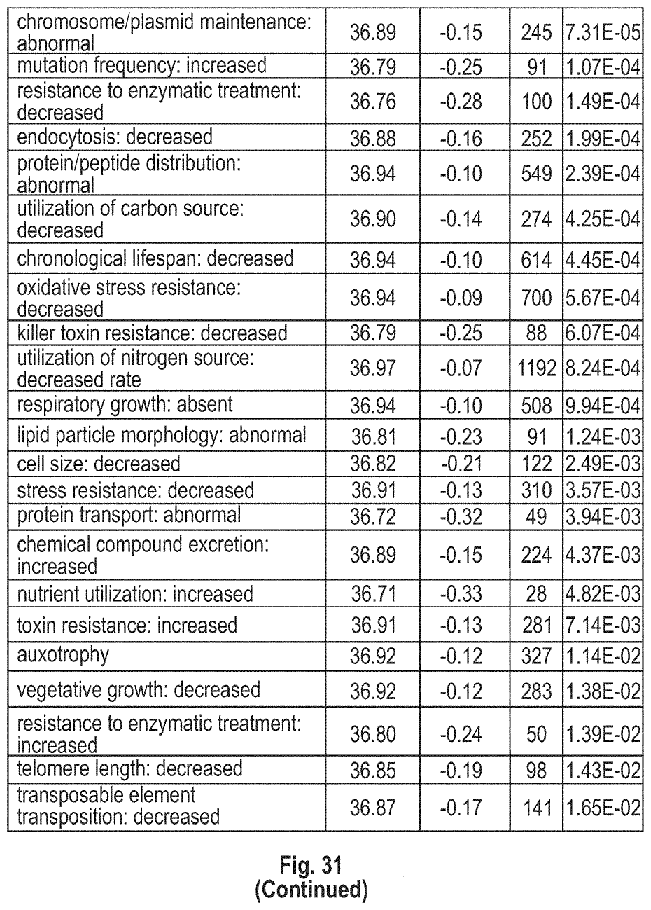

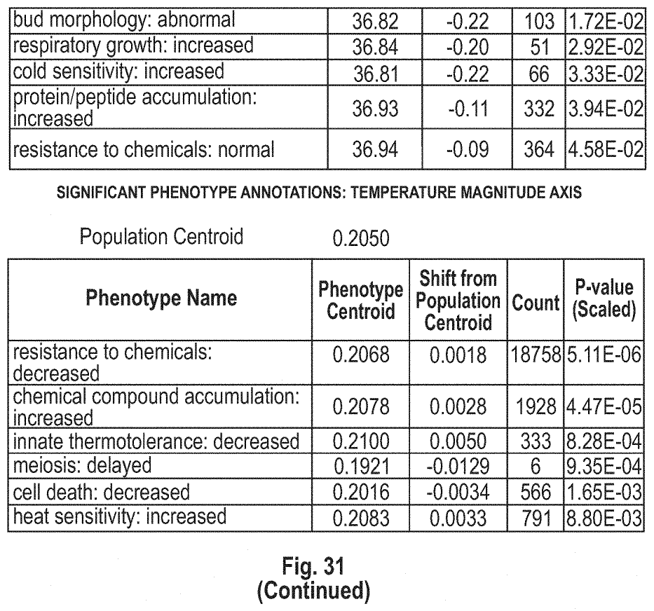

To verify that the fitness centroid metric correlates with the performance of strains across each condition, 100 high-performing members from each condition were highlighted on the centroid distribution map (FIG. 29). For this purpose, high-performing members were defined as those with the largest arithmetic difference in frequency between initial and final timepoints (i.e. freq_{Day 6}−freq_{Day 0}). The centroids of high-performing members clustered in a manner that correlates with the condition in which they were selected, e.g. high performers from the 42° C./48 hr condition cluster in the lower right portion of the graph. Library members with significant fitness centroid shifts along the magnitude or frequency axes were identified (FIGS. 30A-30B), including several chaperone and chaperone cofactor genes, which are known to play a role in thermal stress response (Morano K. A. et al., Genetics 190, 1157-95 (2012)). We also noted significantly shifted library members associated with GO terms identified from principle component analysis described previously (see FIGS. 33A-33C), mainly annotations associated with cell cycle checkpoints (e.g. DNA damage response, organelle fission) which we hypothesized to be involved in frequency response.

Finally, in addition to individual centroid calculations, we also calculated a mean fitness centroid from subsets of the knockout collection annotated on SGD for one of 1011 phenotypes assigned to at least 5 genes. Welch's t-statistic was used to determine whether a subset annotated for a specific phenotype was significantly shifted from the mean centroid of the whole population. In order to account for multiple hypotheses, all p-values were scaled by a factor of 1011. It should be noted that certain phenotype annotations have further sub-annotations (“Resistance to Chemicals” could be further sub-divided by chemical, “Competitive Fitness” could be further subdivided by media condition, etc.) but these sub-annotations were not considered in the present study. Significant phenotype annotations were identified along both the temperature magnitude and temperature frequency axes (FIG. 31). In one broad observation, phenotype annotations were more likely to be significantly shifted towards lower temperature magnitudes (sensitive to high temperatures), than towards either end of the temperature frequency axis. This may be biologically relevant, or simply an artifact of the SGD database, which is dependent both on the topics of study and methods employed by prior researchers. We note that dynamic stresses are rarely annotated in the SGD, suggesting new experimental avenues for assessing gene function and phenotype in platforms and assays that can controllably apply dynamically changing conditions.

Competition Assay to Validate Fitness Centroid Hits:

To validate fitness results from the pooled library screen, we selected four library members from different regions of the fitness scatter plot: ΔHSP104, ΔKAP20, ΔAHA1, and ΔSWA2 (highlighted in red in FIG. 7C). The fitness of each strain was assayed in competition with a neutral control strain, ΔHO strain from the yeast deletion library. The fitness centroid of the ΔHO strain lies close to the mean fitness centroid of the population, indicating it has a neutral effect on the cell with respect to the thermal stresses applied. Furthermore, this deletion would be predicted to have minimal effect on the phenotype of the cell, as the strain used to create the deletion collection contains a nonsense mutation in the HO gene preventing it from forming functional protein even prior to deletion.

These individual deletion members were grown in YPD at 30° C. for 12 h, reaching early stationary phase. Each of the four strains were mixed 1:1 with the ΔHO control strain, then each co-culture was seeded into four eVOLVER vials (containing YPD+50 ug/mL carbenicillin+25 ug/mL chloramphenicol) at OD 0.05. Harnessing the programmable nature of eVOLVER, selection was applied identically as it was for the original pooled experiment. Cells were grown at 30° C. for 5.5 h, and then maintained in continuous turbidostat culture (OD 0.15-0.20). Programmed heat shocks of varying magnitude and frequency were initiated as soon as regular dilution events were underway in all cultures. The control code was slightly modified from the original experiment, such that each of the four co-cultures was exposed to four conditions from the original experiment (33° C./2 h, 33° C./48 h, 42° C./2 h, and 42° C./48 h). Two mL culture samples were taken every 24 h for two days and frozen at −80° C. Genomic DNA was extracted as described previously.

Relative fitness was determined using the frequency of both the strain of interest and the ΔHO strain as determined by qPCR (FIG. 26A). For strain specific amplicons, universal reverse primer prCM314 (targeting a sequence from the deletion cassette downstream of the barcode) was paired with a context specific primer for each particular gene, usually a subsection of the “up45” homology region originally used to create the deletion library (Winzeler E. A., et al., Science 285, 901-906 (1999)). A control amplicon targeted two universal regions of the deletion cassette, primer prCM313 binding upstream of the barcode, and primer prCM317 binding in the resistance marker. The readings from this universal control amplicon were used to normalize readings from the strain-specific amplicons, providing the frequency of each strain in the co-culture. A 1 uL aliquot of genomic DNA extract was used as template for a 20 uL reaction using SYBR Green I Master Mix (Roche) and the aforementioned primers in TABLE 3 using the following cycle conditions: (i) denaturation: 95° C. for 10 min; (ii) amplification (35 cycles): 95° C. for 10 s, 63° C. for 5 s, 72° C. for 14 s; (iii) elongation: 72° C. for 7 min. Although frequencies calculated from primers for each of the four strains were compared to the frequencies calculated from the ΔHO specific primers, only the latter was used for computing fitness values in order to prevent bias due to different primer efficiencies.

Competitive fitness values for each strain of interest were computed using the frequency of the strain, specifically (1−freq_{ΔHO}), in place of a ratio between two strains. The fitness heatmaps created from the qPCR frequencies in the validation study largely agree with the fitness heatmaps created from the sequencing data in the original pooled experiment (FIG. 4D). Of particular note are ΔHSP104 and ΔSWA2, illustrating the drawbacks of the centroid metric in isolation. While both have centroids located in a similar location, the ΔSWA2 centroid is driven largely due to fitness deficits at high temperature, while ΔHSP104 strain exhibits increased fitness at low temperatures.

Catalog of Integrated Millifluidic Devices Developed:

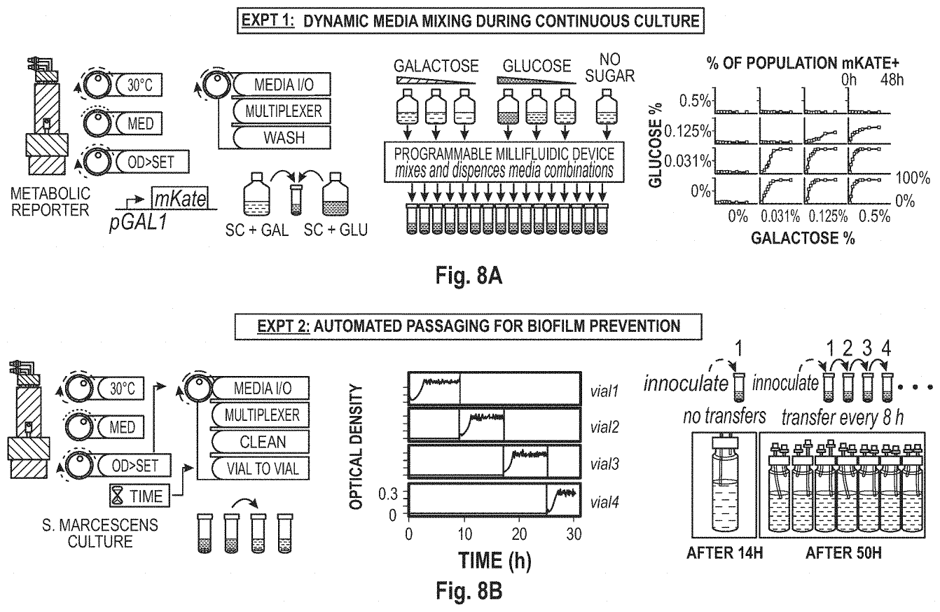

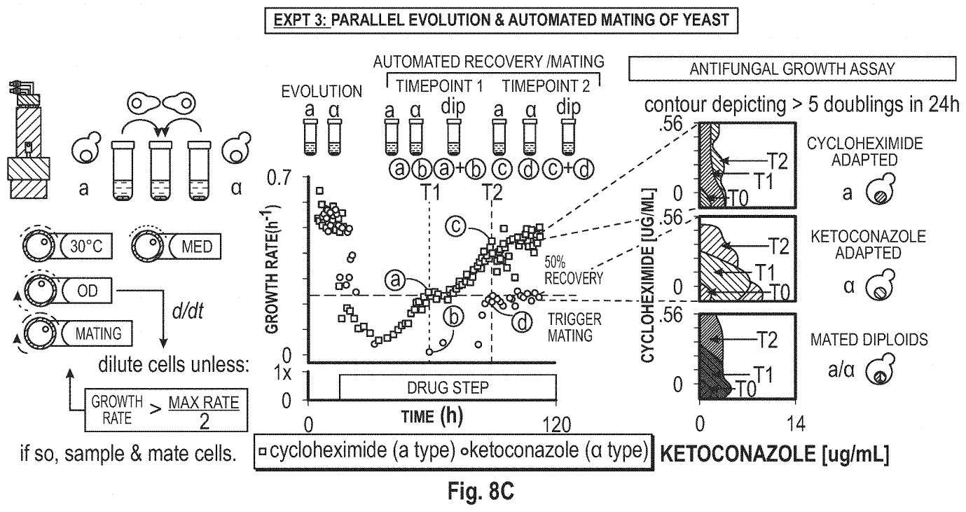

Using the fabrication methods described in Example 15, we developed three different integrated millifluidic devices to carry out complex fluidic tasks for eVOLVER. These devices are showcased in three continuous culture applications with unique requirements for fluidic handling (see FIGS. 8A-8C).

8-Channel Vial Router Device (Used in all Fluidic Demos):

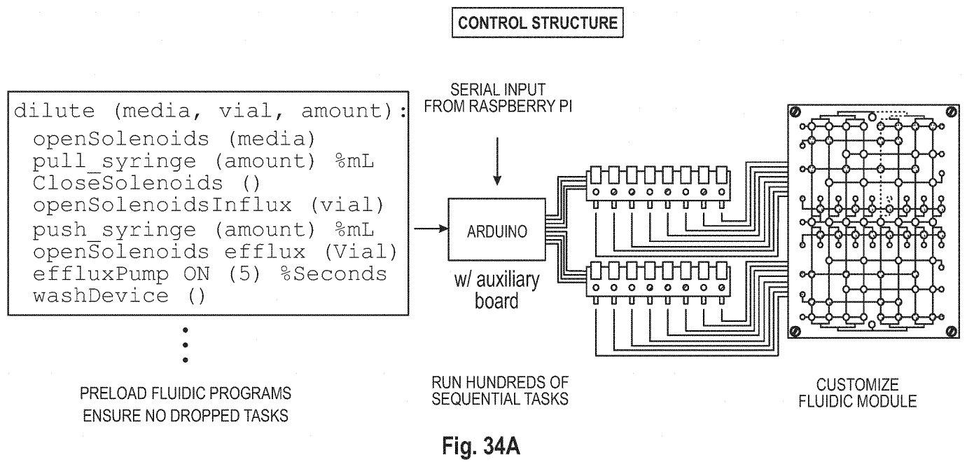

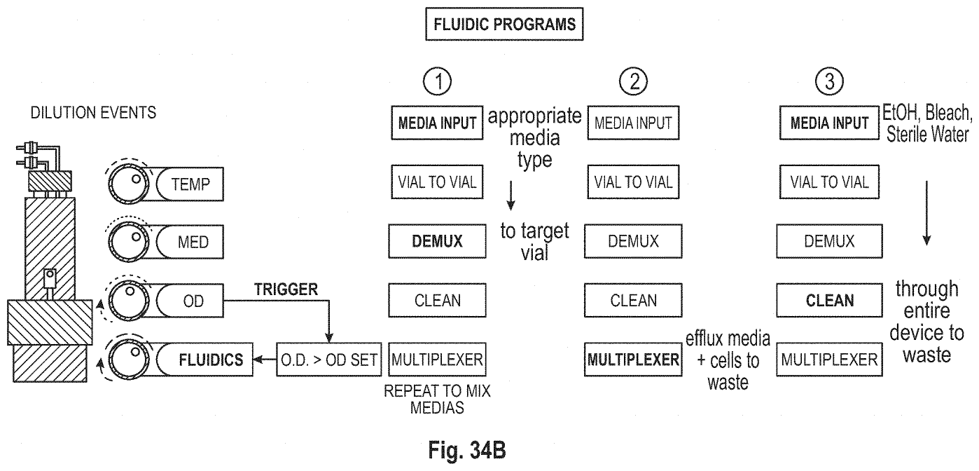

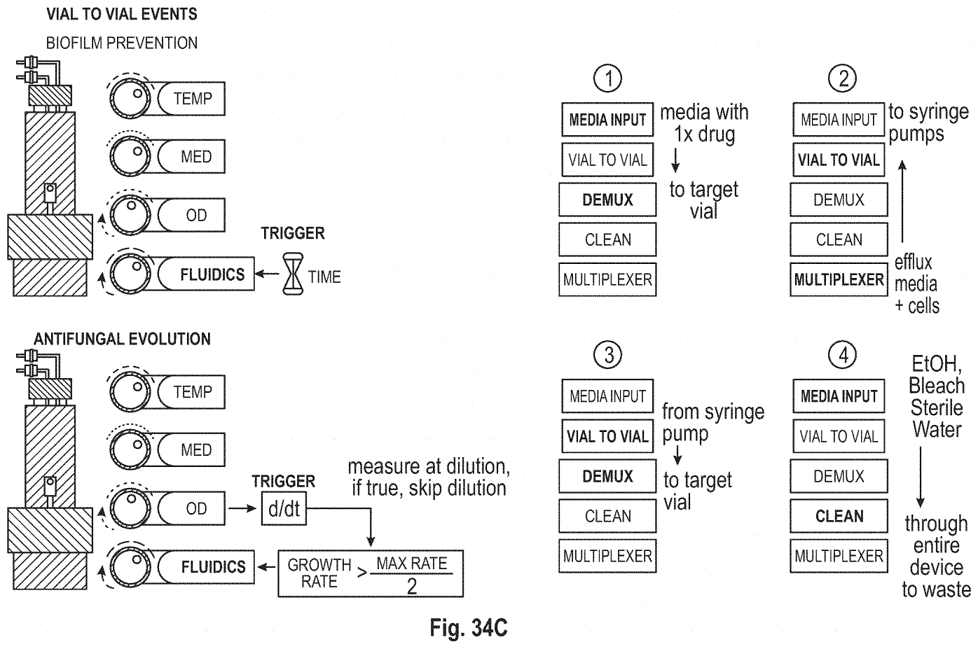

We developed a pneumatic valving schematic that routes fluid to and from eight different vials, termed the 8-channel vial router device (design shown in FIG. 34A). The 8-channel vial router consists of a demultiplexer, which splits a source into 8 channels, influx and efflux ports that am connected to the vials via tubing, a bridge that permits bypassing the vial, and a multiplexer that combines all 8 channels back into one. These segments are consolidated into a common device to minimize necessary fluidic connections between devices. Two 8-channel vial routers are needed to interface with all 16 eVOLVER vials and can be daisy chained together to minimize control elements.

As depicted in FIG. 34A, three paths are available per vial on this device: media in via influx, media out via efflux, and bypass via bridge. The last function is used for washing and rinsing the integrated device without affecting the vial or tubing connecting it to the device. Any routine that interacts with a vial is implemented in part by actuating valving in the vial router device to open the path corresponding to the vial of interest. Each segment of the device can be operated independently. For example, more complex fluidic functions, like vial-to-vial transfer, is enabled by routing the efflux of one vial back to the influx of another vial (see Vial-to-Vial Transfer Device). The vial router device is used in all fluidic demonstrations in FIGS. 8A-8C.

8-Channel Media Selector Device (Used in Dynamic Media Mixing Demo):

The next integrated device, the 8-channel media selector, was developed in order to permit media mixing via sequential actuations of a syringe pump. As depicted in FIG. 34B, the media selector consists of three main components: an 8-channel input multiplexer, a syringe pump port, and valves to select between two 8-channel vial router devices. In more detail, the integrated multiplexer chooses between 8 possible fluid inputs (air and 7 media types) to be fed into the vial router devices. Since the two vial router devices are daisy chained (share the same solenoid control lines), the additional valves described are added to differentiate between the two possible routes.

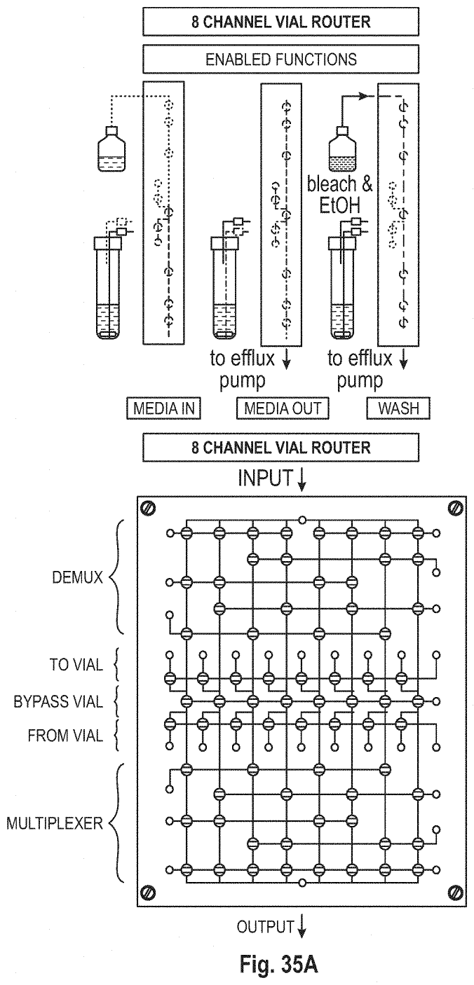

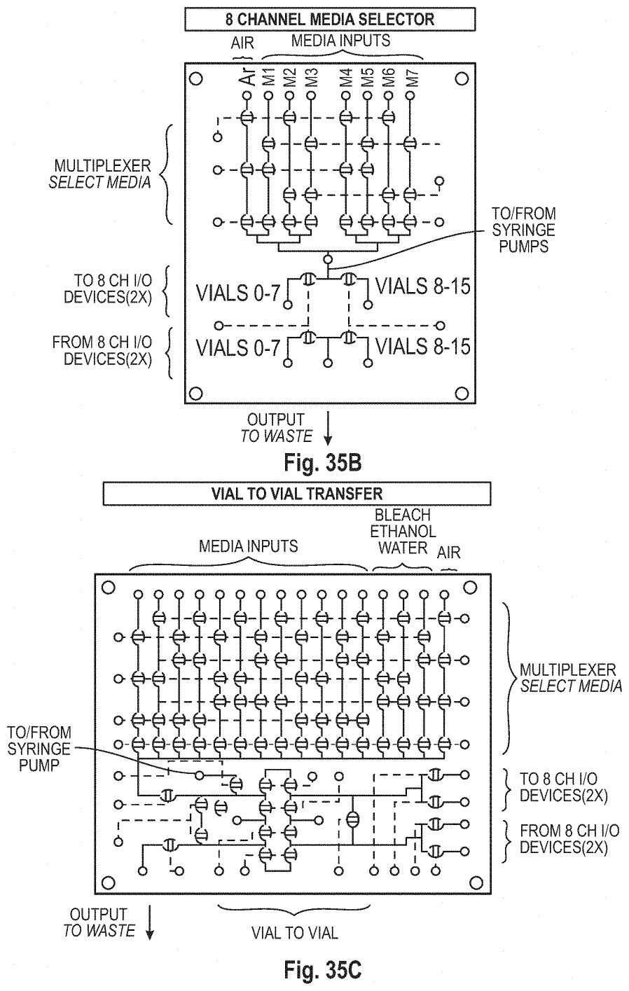

In the ratio sugar sensing demonstration (FIG. 8A), this device was used to execute dilution events requiring mixing media sources (refer to FIG. 34B) for logic diagram for this programmed routine). For the influx portion of a dilution event, one or more medias are sequentially drawn into the syringe by opening different paths in the input multiplexer, then dispensed into a vial through the demultiplexer of one of the vial router devices as depicted in FIG. 35A. For the efflux portion, a path through the multiplexer of the vial router device is opened, then a peristaltic pump pulls efflux media out through the main waste line. Finally, to rinse the device, the wash fluid (sugar-free SC media, in this example) is used to flush the syringe and main paths of the media selector, as well as the channel used in the vial router device. For this combination of devices, 25 control elements are required: 15 valves to control the vial router devices. 8 for the media selector, 1 actuator for the syringe pump, and one for the peristaltic pump in the main waste line. This amounts to only half of the 48 channels available on the auxiliary board.

Vial-to-Vial Transfer Device (Used in Biofilm Prevention and Yeast Mating Demos):

The final integrated device developed in this study, the vial-to-vial transfer device permits media transfer of culture from any one eVOLVER vial to any other. In order to maintain sterility within the device, expanded cleaning options were needed as well. As depicted in FIG. 35C, the vial-to-vial transfer device consists of five main components: a 16-channel input multiplexer, the efflux-to-influx bridge (enables vial-to-vial), a syringe pump port, a waste port, and valves to select between 8-channel vial router devices (similarly described in the media selector device section). Additionally, several additional valves were placed in critical locations throughout the device to ensure no contamination due to backflow. On this device, one can choose between 16 fluidic inputs (12 media inputs, 3 for sterilization, and 1 air). Note that with the exception of the efflux-to-influx bridge, this device is essentially an expanded version of the multiplexed media selector described above, and can carry out all the functions that were possible in the smaller device. Also note the exponential scaling of inputs, to go from 8 input channels to 16 input channels only requires two additional control elements (going from 6 to 8).

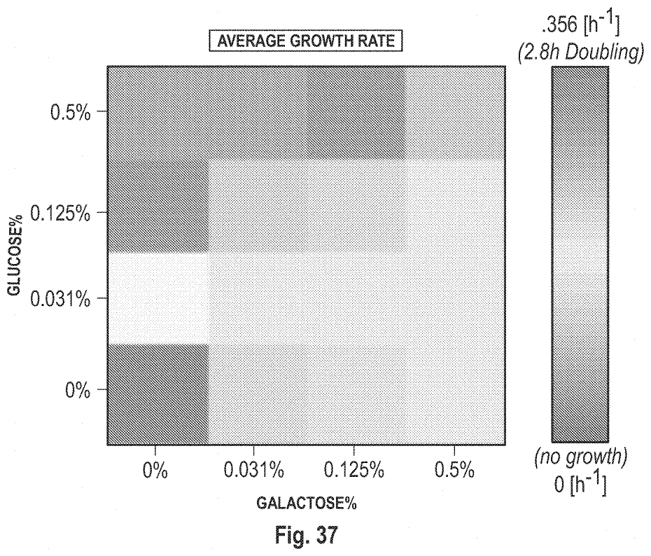

In the automated passaging biofilm prevention (FIG. 8B) and parallel evolution and mating (FIG. 8C) demonstrations, this device was used to execute vial-to-vial transfer events (refer to FIG. 34C for the logic diagram for a typical example of this programmed routine). For the source vial, first media is drawn into syringe through the multiplexer, then dispensed into the source vial through the demultiplexer of one of the vial router devices, then culture is pulled through the efflux line into the syringe as depicted in FIG. 37. For the target vial, the culture sample is dispensed through the demultiplexer of a vial router device. Finally, the device is thoroughly sterilized by washing the syringe, the vial router devices, and the entire vial-to-vial transfer device first with 10% bleach, followed by ethanol, then rinsed with sterile water. For this combination of devices, 38 control elements are required: 15 valve actuators to control the vial router devices, 21 for the vial-to-vial transfer device, 1 actuator for the syringe pump, and 1 actuator for the main waste pump. This amounts to just over ¾ of the 48 channels available on the auxiliary board, indicating that even more complex fluidic functions are accessible.

Abstract Commands to Automate Complex Fluidic Routines:

As evidenced by the complex descriptions of the fluidic routines above, there is a need for abstraction when sending commands to the integrated fluidic devices. Enumerating specific control elements seems feasible when dealing with a simple peristaltic array. However, this is extremely tedious when dealing with the valving schemes of integrated devices. Additionally, as numerous sequential events are often needed to carry out fluidic tasks in the integrated devices (e.g.: open valves, then pull syringe, then change valves, then dispense syringe), robust transition between sub-tasks is needed. A missed step could lead to mis-priming the syringe pump or incorrectly routing fluid into the wrong location. Both of these concerns are addressed with abstract fluidic routines encoded as scripts and functions on the Arduino microcontroller (FIGS. 34A-34C). This means that an abstract serial command, “dilute vial 3 with media input B”, can be issued by the user in the Python script but translated by the Arduino into a series of actuation events, which are rapidly carried out without the need for multiple rounds of communication between the computer and the Raspberry Pi (often the rate limiting step for other functions).

Dynamic Media Mixing for Ratio Sugar Sensing:

In order to demonstrate that fluidic multiplexing could be used to manage media composition for multiple cultures maintained by eVOLVER, we constructed an 8-channel media selector device that dynamically draws media from multiple input sources and addresses a defined mixture to a culture of choice. We used this to interrogate and characterize yeast galactose metabolic gene induction, which is known to respond to ratios of galactose and glucose (Escalante-Chong R., et al., Proc. Natl. Acad. Sci. U.S.A 112, 1636-41 (2015)) (FIG. 8A).

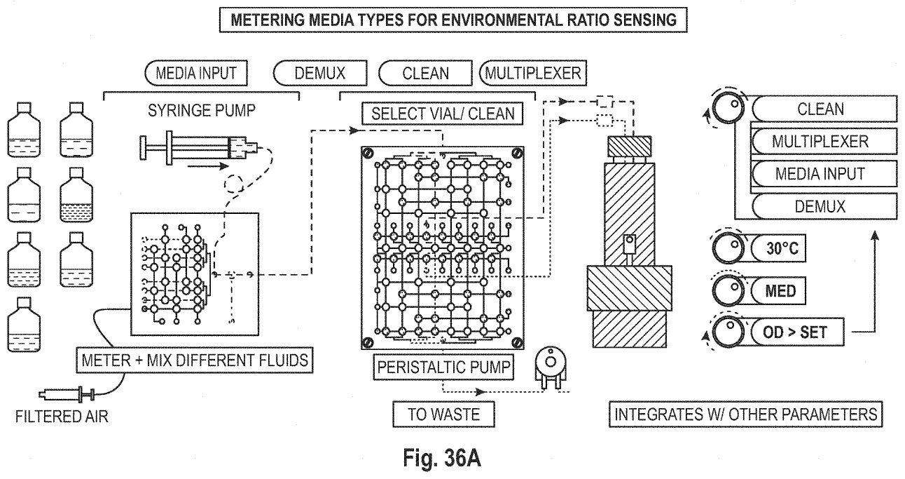

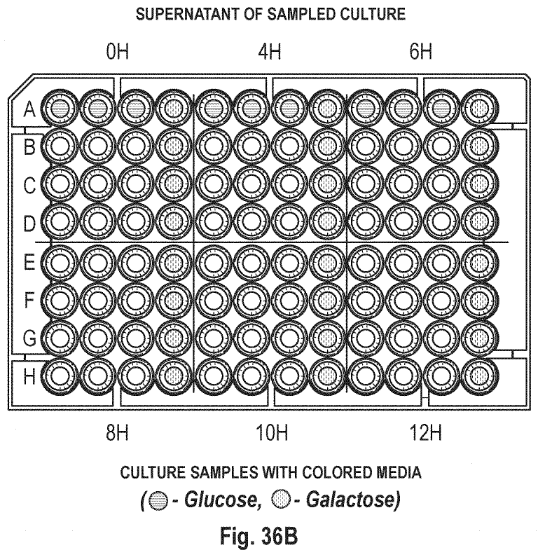

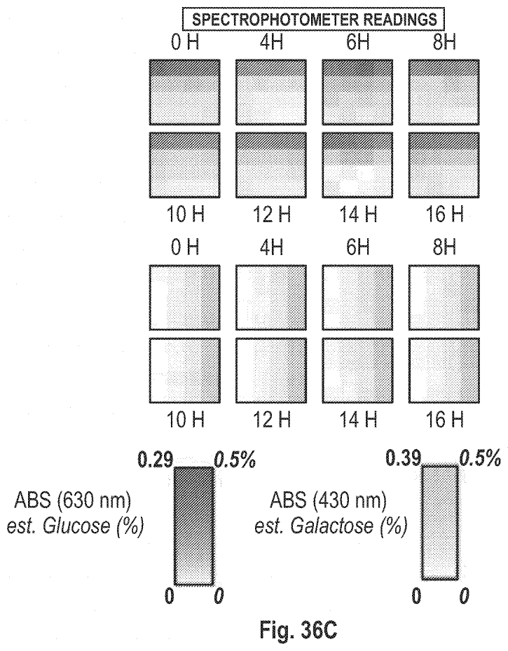

Glucose and galactose solutions were labelled with blue and yellow food coloring, respectively. These solutions were then used to supplement SC media (also supplemented with 50 mg/mL adenine hemisulfate) in a 4-fold dilution series of each sugar type (1%, 0.25%, and 0.06375%). We measured the 6 resulting medias and a sugar-free control on a Spectramax M5 plate reader spectrophotometer, using absorbance at 630 nm and 430 nm to estimate the component sugar concentrations independently (FIGS. 36A-36C).

For the first experiment, yeast cells harboring an integrated galactose-inducible reporter (pGAL1-mKate2, ySK499) were grown from frozen stocks in YPAD (YPD+50 mg/mL adenine hemisulfate) overnight, then diluted 1:100 into flasks containing SC+2% raffinose+50 mg/mL adenine hemisulfate and grown for 16 h in a shaking incubator at 30° C. We prepared seven different medias using color-labelled sugars as before: three SC+glucose medias (1%, 0.25%, and 0.06375%), three SC+galactose medias (1%, 0.25%, and 0.06375%), and a SC sugar-free control. By mixing any two of the seven medias, we could create 16 different SC+sugar compositions: three glucose-only (at 0.5%. 0.125%, 0.031875%), three galactose-only (at 0.5%. 0.125%, 0.031875%), nine different glucose/galactose ratios, and a sugar free control.

Yeast cultures were maintained in eVOLVER at the specified sugar compositions at a density window of OD 0.2-0.3 for 16 h. This was achieved using the 8-channel media selector device (FIGS. 34A-34C and FIGS. 35A-35C) to dynamically mix together the appropriate two medias for each vial at each dilution event. Culture samples were collected at regular timepoints, centrifuged at 1000 rcf for 5 min to pellet cells, and the supernatant was measured in a spectrophotometer as described above to estimate sugar concentrations. We observed that sugar ratios were maintained correctly over the course of the experiment, confirming that the syringe pump and lines were effectively cleaned by the cleaning routine, and that pneumatic control valves did not leak despite continual use over an extended time (FIGS. 36A-36C).

The second experiment was performed as above, but food coloring was excluded from the media so as to not affect cell growth in any way. Yeast cultures were prepared and seeded into eVOLVER vials as before, and maintained at the specified glucose/galactose ratio at a density window of OD 0.2-0.3 for 36 h. Culture samples were taken every 2 h for 16 h (with additional steady state timepoints taken at 24 and 36 h) to determine the induction rate of the galactose reporter. Flow cytometry was performed on fixed samples, and the percentage of mKate2+ cells was calculated by gating cells with mKate2 fluorescence higher than the t=0 control (representing the uninduced state in raffinose) (FIG. 8A). While the ratio-dependence of galactose regulation is well established (Escalante-Chong R., et al., Proc. Natl. Acad. Sci. U.S.A 112, 1636-41 (2015)), continuous culture proved valuable to demonstrate the behavior and steady state induction level of the system at maintained glucose and galactose concentrations (in contrast to batch culture experiments). Growth rate was also measured continuously in order to determine a mean growth rate in each media combination (FIG. 37). Interestingly, growth rate was found to be not only a function of the total sugar content, but also of the glucose/galactose ratio. On the whole, cells that are utilizing galactose—evidenced by induction of the pGAL1 reporter—appear to grow slower than cells using glucose, consistent with single cell data gathered with microfluidics (Nguyen-Huu T. D., et al., PLoS Comput. Biol. 11, e1004399 (2015)).

Automated Passaging for Biofilm Prevention:

To demonstrate the utility of the millifluidic system for mediating liquid transfer between cultures, we designed a device capable of overcoming biofilm formation during long-term continuous growth experiments. To do so, we applied the vial-to-vial transfer device to continually passage cells into fresh culture vessels in an automated fashion (FIG. 8B).

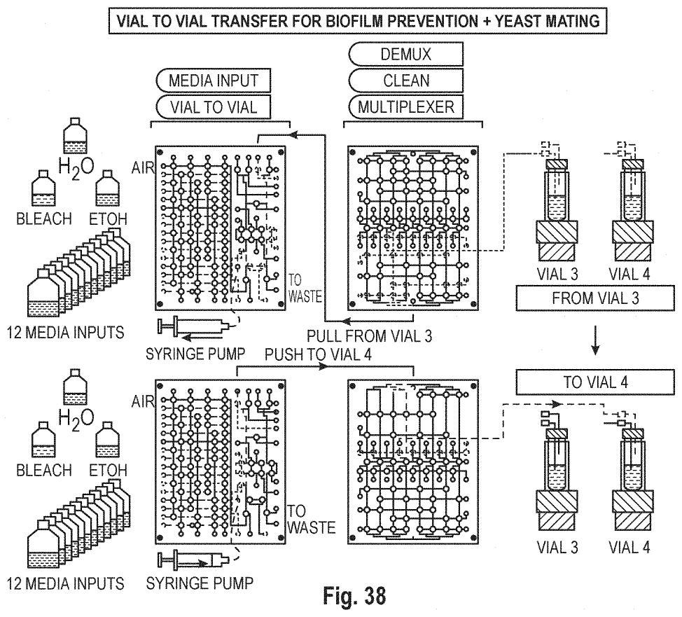

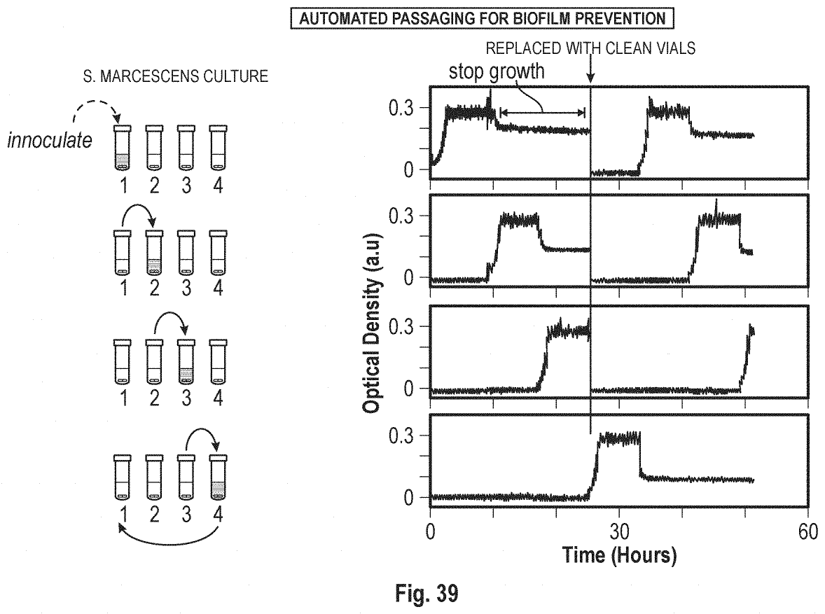

An overnight culture of Serratia marcescens (ATCC 13880) was grown in pre-buffered LB Miller media (pH 7.2). This culture was used to seed two eVOLVER vials at OD 0.05. A control vial was maintained in a density window of OD 0.25-0.3 at 30° C. for 14 h before enough biofilm had deposited to affect density measurements. The second vial was grown for 9 h, at which point a 2 mL aliquot was transferred to a new vial containing fresh media using the automated vial-to-vial transfer device (Methods and FIG. 38 and FIG. 39). This transfer was repeated every 8 h for a total of 48 h into fresh vials. Every 24 h, spent vials were replaced with fresh vials in order to reuse the eVOLVER sleeves in a cyclical manner. An automated sterilization protocol was run in the device following each transfer, wherein all affected fluidic lines were flushed with a 10% bleach solution, a 70% ethanol solution, and finally sterile water. A 5 mL aliquot of 10% bleach was also automatically added to the source vial in order to halt further growth. Vials were photographed at the conclusion of the experiment (FIG. 8B).

While simple in design, this result is impactful for enabling continuous culture for undomesticated microbes that have proved incompatible with routine continuous culture. It provides a non-chemical means to prevent biofilm, preventing possible toxic or other unintended effects on vegetative growth in the culture. This approach also flips the selection for biofilm in traditional continuous culture systems by, in fact, selecting against adherent cells over time. This mechanism has been implemented by a few single-purpose devices (de Crdcy-Lagard V. A., et al., BMC Biotechnol. 1, 10 (2001); de Crécy E., et al., Appl. Microbiol. Biotechnol. 77, 489-496 (2007)), but the flexibility of the eVOLVER platform permits other types of manipulations due to the customizable nature of the millifluidic devices. Finally, while the present experiment could be achieved through manual transfers, the benefits of an automated system are realized for longer-term experiments in which frequent manual transfers (every 8 h for this species) become burdensome.

Parallel Yeast Evolution and Mating in Automated Cell Culture:

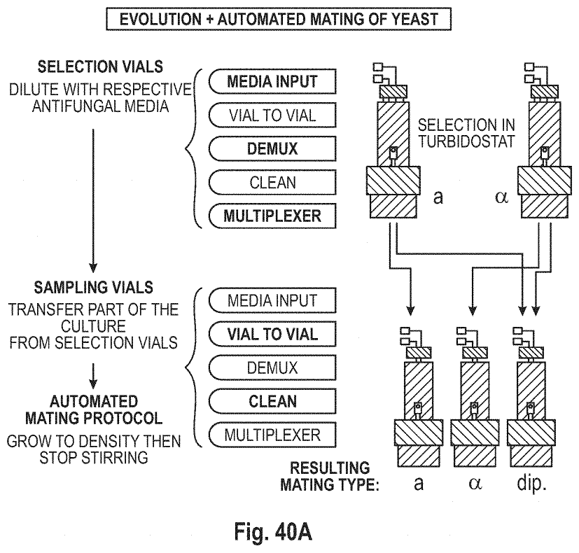

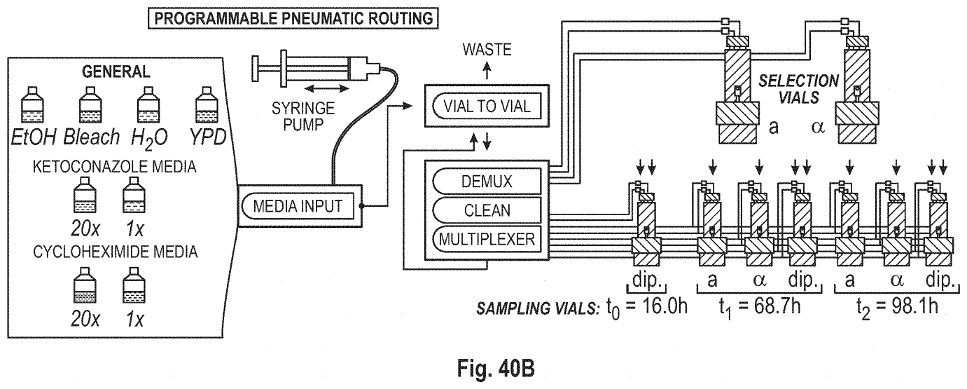

Finally, we sought to apply millifluidic multiplexing in an experiment requiring coordination of multiple fluidic functions in an automated fashion. We sought to carry out parallel evolution in two haploid yeast populations, and programmatically mate cells at biologically relevant timepoints to harness sexual reproduction as a trajectory for adaptation (McDonald M. J., et al., Nature 531, 233-236 (2016)) (FIG. 8C). Such an experiment required multiplexed media selection, vial-to-vial transfers, and cleaning (FIGS. 40A-40B) to all be carried out in a single integrated device, demonstrating that these devices can enable novel automated cell culture experiments.

Automated Yeast Mating Routine:

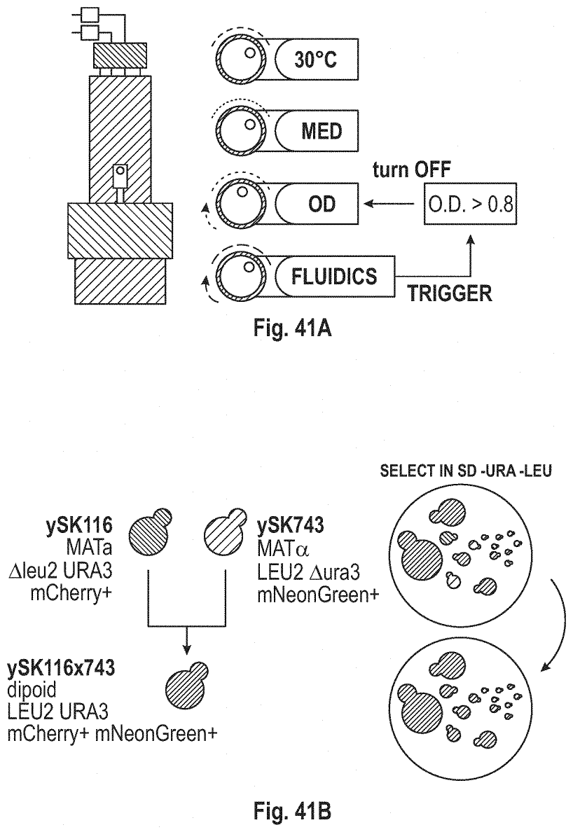

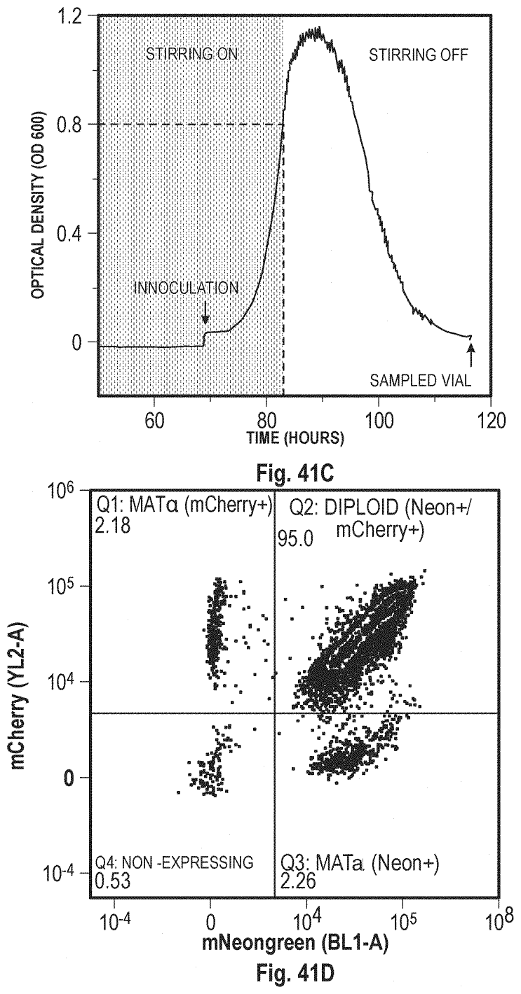

Overnight cultures of fluorescently labelled haploid MATα (ySK116) and fluorescently labelled haploid MATα (ySK743) cells were grown overnight in YPD (FIGS. 41A-41D). These overnights were used to seed two eVOLVER vials at OD 0.05 (containing YPD+50 ug/mL carbenicillin+25 ug/mL chloramphenicol). Cells were maintained at 30° C. in a density window of OD 0.25-0.3 for several generations. Next, the automated vial-to-vial transfer device was used to transfer 2 mL from each haploid culture into the same vial. This co-culture was grown at 30° C. with constant stirring, but no dilutions, until reaching a density of OD 0.8, thereupon stirring was halted, allowing cells to settle to the bottom of the vial at high density. After density readings had dropped to OD 0.1 (˜20-36 h after stirring stopped), the vial was removed from the device, cells were resuspended by shaking, then a 30 uL aliquot was diluted 1:100 into SD-Ura-Leu (supplemented with 2% glucose and 50 mg/mL adenine hemisulfate) selection media and grown for 24 h to enrich for diploids (FIGS. 41A-41D). The purity of diploids following selection was determined using flow cytometry to quantify the proportion of cells expressing both fluorescent labels vs. a single label (FIGS. 41A-41D). While haploids of each type do leak into the final population even after selection, this could easily be addressed with a lethal selection scheme (McDonald M. J., et al., Nature 531, 233-236 (2016)), such as expression of resistance genes for hygromycin, nourseothricin, or G418. However, in the present work we retained auxotrophic selection to prevent confounding factors as we applied eVOLVER and the vial-to-vial transfer device to evolve resistance to two antifungals in parallel.

Parallel Evolution of Antifungal Resistance and Mating:

Single colonies of fluorescently labelled haploid MATα (ySK116), fluorescently labelled haploid MATα (ySK743), and fluorescently labelled diploid control (ySK116x743) cells were grown overnight in YPD. These overnights were used to seed three eVOLVER vials at OD 0.05 (containing YPAD+50 ug/mL carbenicillin+25 ug/mL chloramphenicol). Cells were maintained at 30° C. in a density window of OD 0.25-0.3 for several generations in order to measure a baseline growth rate. At 16 h post-inoculation, cells from each haploid population were transferred by the vial-to-vial transfer device into the same fresh vial containing YPAD in order to create a new pre-drug to diploid population.

Next, cyclohexamide (CHX) was added to the haploid MATα vial, ketoconazole (KETO) was added to the MATα vial, and both drugs were added to the diploid control. Using the media selection portion of the vial-to-vial transfer device, 1 mL aliquots of media at 20× drug concentration were used to achieve a step-function transition to the final 1× drug concentration in each vial (0.2 ug/mL CHX for MATα, 6 ug/mL KETO for MATα, 0.2 ug/mL CHX+6 ug/mL KETO for the diploid control). Growth rate was continuously tracked, and was found to drop to roughly 10% of its original value over the 20 h following drug addition (FIG. 8C). As reported in previous studies, the KETO-exposed culture was observed to undergo a period of growth seemingly unaffected for a few generations before slowing (Anderson J. B., et al., Genetics 168, 1915-23 (2004)). While the diploid control exposed to the combination of CHX and KETO eventually slowed to a hall, exhibiting no bulk growth at all (data not shown), each haploid population eventually began to recover and increase in growth rate (FIG. 8C).

Two automatically triggered timepoints were taken: t₁ (or “CHX recovery”) was triggered at 68.7 h by the MATα vial returning to 50% of its pre-drug growth rate; and t₂ (or “KETO recovery”) was triggered at 98.1 h by the MATα vial returning to 50% of its pre-drug growth rate (FIG. 8C). For each timepoint, three vials (containing YPAD+50 ug/mL carbenicillin+25 ug/mL chloramphenicol, but no antifungals) were inoculated by vial transfers: one with treated MATα haploids only, one with treated MATα haploids only, and one with both in order to create diploids (FIGS. 40A-40B). Each of the timepoint cultures was grown to OD 0.8, followed by a period of settling (see Automated Yeast Mating Routine). After waiting to allow sufficient cell settling (˜20-36 h after stirring stopped), a 700 uL aliquot of each bulk population was mixed with 300 uL of 50% glycerol and stored at −80° C. Simultaneously, 30 uL aliquots were diluted into 3 mL of liquid selection media (SD-Ura for MATα, SD-Leu for MATα, or SD-Ura-Leu for diploids) and grown for 16 h, then mixed with glycerol and frozen as before. Cells were additionally streaked onto solid selection agar (SD-Ura for MATα, SD-Leu for MATα, or SD-Ura-Leu for diploids) in order to isolate single colonies for sequencing. Diploids from liquid selection and solid selection were confirmed by flow cytometry.

This experiment highlights another one of the advantages of an automated system over manual timepoints, as addressed in the automated passaging for biofilm prevention experiment. While manual sampling is easy for short routine experiments, automated sampling is extremely valuable over a long experiment when timepoints are frequent or unpredictable. Programming the sampling logic permitted us to sample cells at biologically-motivated timepoints (50% growth recovery) rather than arbitrary, schedule-motivated timepoints. Between remote real-time monitoring and programmable manipulations, the amount of time spent physically overseeing an experiment can be greatly reduced.

MIC Assay to Evaluate Antifungal Resistance:

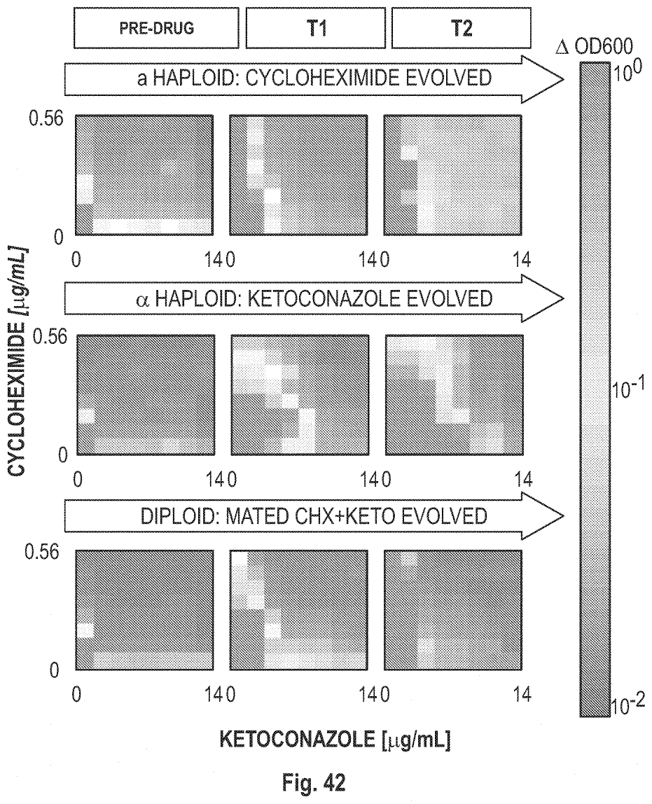

To evaluate the degree to which evolved strains and the resulting diploids were resistant to each drug in isolation or combination, a variant Minimum Inhibitory Concentration (MIC) assay was performed on cells from each timepoint. Nine samples were across 64 combinations of cyclohexamide and ketoconazole: founder ySK116 and MATα samples from each timepoint: yBW004, yBW005; founder ySK743 and MATα samples: yBW006, yBW007; and the three diploid samples: yBW003, yBW008, and yBW009; see TABLE 2). 100 uL of each frozen stock created from post-selection cultures was thawed, added to 2 mL YPAD and then grown in culture tubes in a shaking incubator for 16 h at 30° C. Linear dilution series were prepared for each drug at 4× concentration in YPAD. In 96-well deep well blocks, 100 uL of each drug media was added to 200 uL of cells to bring every component to desired final concentration (0-0.56 ug/mL CHX, 0-14 ug/mL KETO, at OD 0.01). Each well received an estimated 60,000 cells from the non-clonal population that comprised each sample pool. The resulting 400 uL cultures were grown in a shaking incubator at 900 rpm and 30° C. for 24 h, then 200 uL of each culture was measured on a Spectramax M5 plate reader spectrophotometer. A blank measurement of cells at the seeding density of OD 0.01 was subtracted from the endpoint measurements to compute the change in optical density resulting from growth (FIG. 42). This data can be summarized with contours delineating the region corresponding to an OD≥0.32, roughly 5 generations (FIG. 8C).

As expected, each haploid population developed a different antifungal resistance phenotype. Intriguingly, the CHX evolved pools exhibit a strong resistance phenotype that is specific to CHX, while the KETO evolved pools have a milder, more generalized resistance phenotype. Additionally, while CHX resistance is clearly passed on to the diploid pool, suggesting a dominant mutation, KETO resistance is not passed on, suggesting a recessive mutation in the haploids. There are numerous mechanisms by which resistance to either drug may be achieved (Anderson J. B., et al., Genetics 168, 1915-23 (2004); Kanafani Z. A. and Perfect J. R., Clin. Infect. Dis. 46, 120-128 (2008)). For the present study, we explored two possible avenues (see below).

It should be noted that as measurements were performed on pooled samples containing non-isogenic populations, the results may be influenced by the distribution of resistant cells in the population in addition to the resistance of any particular cells. While not a true MIC assay in the traditional sense, this assay still provides valuable information about the performance of the evolving populations over time.

We also note that cell density dependence is commonly observed in antibiotics and antifungals (Brook I., Rev. Infect. Dis. 11, 361-8)). This is likely responsible for the apparently contradictory result that the evolution experiment was performed at drug concentrations above the measured MIC of the founder strains. It also suggests additional utility for density tracking in eVOLVER, permitting cultures to be assayed for resistance to antibiotics, antifungals, or other stressors under conditions of tightly controlled density in a replicable manner.

Growth Rate Assay to Evaluate Antifungal Resistance:

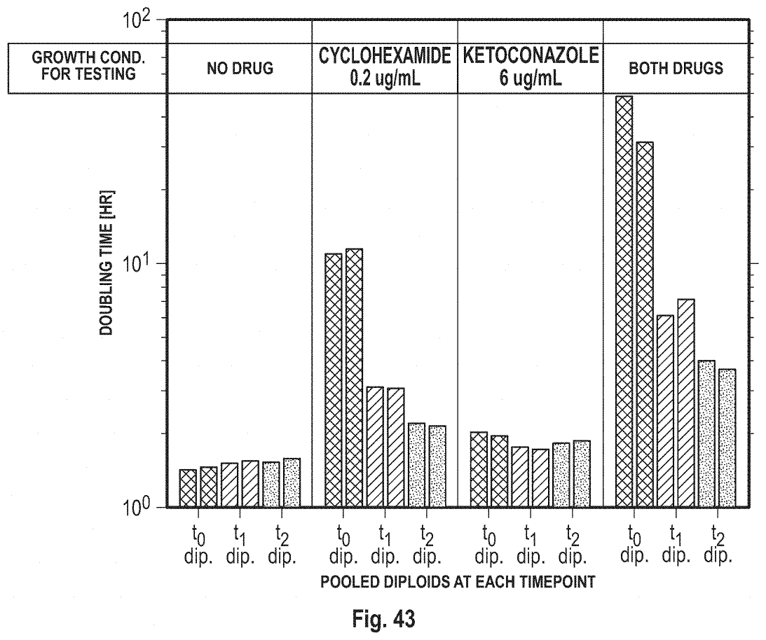

As an alternative metric to evaluate performance of the diploids formed during the experiment, a growth rate assay was performed in media containing each drug in isolation or combination. Three diploid pool samples were assayed: yBW003 from to, yBWOO8 from t₁, and yBW009 from t₂. 100 uL of each frozen stock created from post-selection cultures was thawed, added to 2 mL YPAD and then grown in culture tubes in a shaking incubator for 16 h at 30° C. These cultures were used to seed duplicate eVOLVER vials containing media with one of four drug conditions (no drug, 0.2 ug/mL CHX only, 6 ug/mL KETO only, both 0.2 ug/mL CHX+6 ug/mL KETO) at an initial density of OD 0.1. Each vial received an estimated 3.2×10⁷ cells from the non-clonal population that comprised each sample pool. Cultures were maintained in eVOLVER at 30° C. for 24 h without dilution, while tracking density continuously. Growth rate was calculated for each culture over the density range OD 0.2-0.8 (FIG. 43). For poorly growing cultures that did not reach OD 0.8, growth rate was fit on a reduced range in which growth was observed. As the cells were seeded at moderate density OD 0.1, KETO was not observed to significantly affect the growth rate during the course of this growth rate experiment, consistent with previous work suggesting that several generations of growth in KETO are required before growth inhibition takes effect (Anderson J. B., et al., Genetics 168, 1915-23 (2004)). Despite this limitation, the CHX and combination drug treatments do exhibit reduced doubling times for diploids formed at later timepoints, compared with yBW003 formed from the founder haploids before drug addition.

Sequencing Antifungal Resistance Mutations:

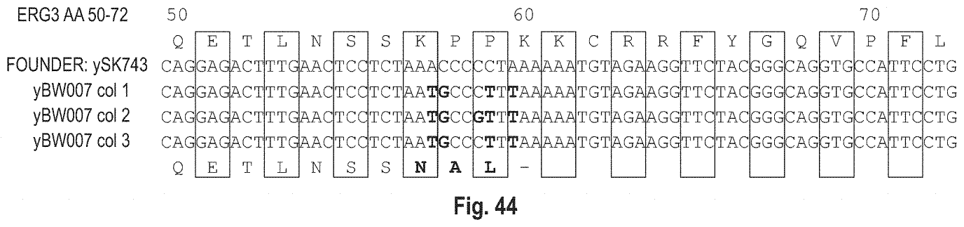

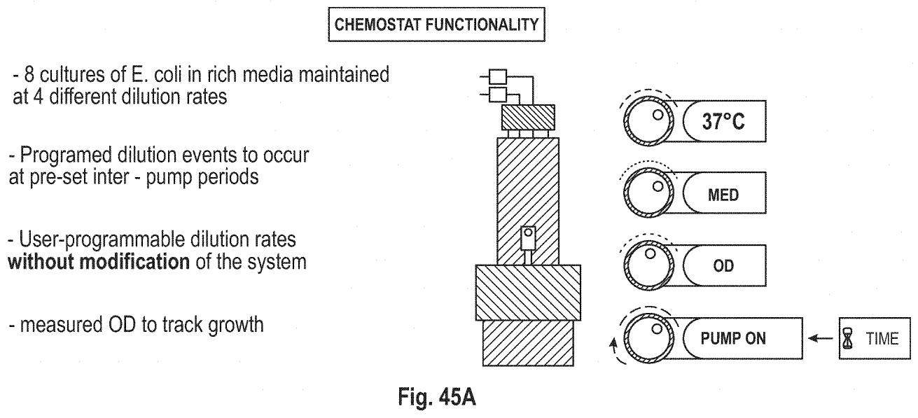

We sequenced one potential mutational target for each drug in the evolved haploid lines. Primers were designed to sequence RPL41A/RPL41B, two paralog genes encoding the molecular target of CHX (Cokol M., et al., Mol. Syst. Biol. 7, 544 (2011)), and ERG3, encoding an enzyme that can confer resistance to azoles when mutated (Kanafani Z. A. and Perfect, J. R., Clin. Infect. Dis. 46, 120-128 (2008)). Genomic DNA was extracted from 100 uL aliquots of the following cultures: 1) MATα founder (ySK116), 2) MATα founder (ySK743), 3) three resistant clones from the CHX-evolved MATα population (yBW005), and 4) three resistant clones from the KETO-evolved MATα population (yBW007). The target genes were isolated from genomic DNA extracts by PCR using primers prCM353-360 (TABLE 3) in a 20 uL reaction with q5 polymerase (New England Biolabs) with the following cycling conditions: (i) denaturation: 95° C. for 10 min; (ii) amplification (30 cycles): 95° C. for 10 s, 62° C. for 10 s, 72° C. for 40 s; (iii) elongation: 72° C. for 7 min. Resulting DNA was purified using a DNA Clean Concentrator Kit (Zymo Research), and Sanger sequencing was performed. All three yBW005 colonies were found to have the same RPL41A/RPL41B sequences as the ySK116 founder, indicating that resistance is gained via a different mechanism. However, mutations in ERG3 were detected in all three yBW007 colonies, notably a nonsense mutation in ERG3 at amino acid 60 (FIG. 44); ERG3 loss of function mutations have previously been shown to confer resistance to azoles in a recessive manner (Anderson J. B., et al., Genetics 168, 1915-23 (2004)), suggesting that the mutations observed contribute to the ketoconazole resistance observed in our populations.

Chemostat Function Demonstration:

We sought to demonstrate that eVOLVER may be operated under different continuous culture regimes simply by changing the Python script. To do so, we devised and carried out a simple chemostat experiment without any modification to the hardware or Arduino scripts.

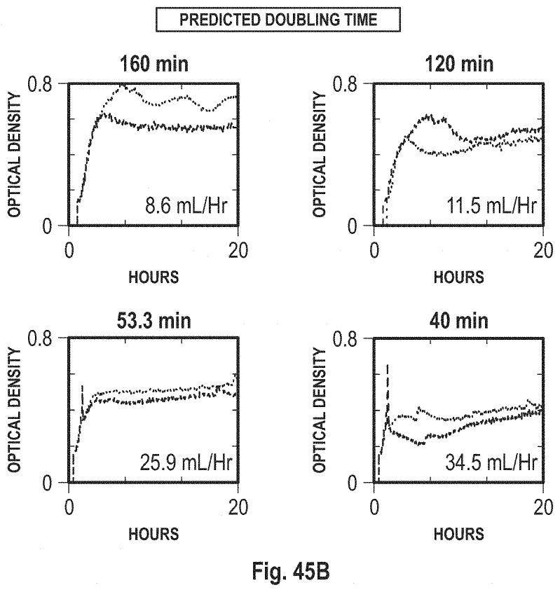

An overnight culture of Escherichia coli TG was grown in pre-buffered LB Miller media (pH 7.2). This culture was used to seed 12 eVOLVER vials at initial OD 0.05. The vials were maintained at moderate stir rate at 37° C. for 2 h before programmed dilutions were initiated. eVOLVER was programmed to continually dilute vials at six pre-set dilution intervals in duplicate. Pump intervals (2-8 min) and pump duration (1-4 s=1.15-4.6 mL) were set to correspond to dilution rates of 6 hr⁻¹, 3 hr⁻¹, 1.5 hr⁻¹, 1.125 hr⁻¹, 0.5 hr⁻¹, and 0.375 hr⁻¹. Density was tracked continuously for each replicate culture and overlaid (FIGS. 45A-45B). The doubling times required to survive each dilution rate are 10, 20, 40, 53.3, 120, and 160 min, respectively, with the first two being faster than the observed doubling time of our E. coli strain in these media conditions, leading to rapid disappearance of cells on the density trace (data not shown). This shows that eVOLVER is capable of performing as a continual-dilution chemostat over a range of dilution rates that are biologically relevant. Combined with remote real-time monitoring, eVOLVER can be used to maintain a culture at constant dilution rate while keeping track of density to identify events that may cause rapid change in growth rate, such as mutation or contamination.

While this experiment was designed to demonstrate that selection conditions can be changed with changes to the Python code alone, this is not the only way to enable chemostat functionality. In fact, communication burden can be reduced by abstracting repeated dilution routines by altering code on the auxiliary board Arduino controlling the pumps. This strategy has proved robust in preliminary work.

### Example 6. eVOLVER: A Scalable Automated Cell Culture Framework

Most automated culture systems are designed to address specialized needs and are thus limited to active control over one or more predetermined culture parameters. We designed eVOLVER so that it could be flexibly configured to measure and control an arbitrary, user-defined set of parameters. In order to accomplish this, the system's hardware design emphasizes rapid, cost-effective scaling and customization, as well as accommodation of future technological advancements (FIG. 4C). eVOLVER hardware includes the following three modules (FIG. 4D): (1) customizable Smart Sleeves, which house and interface with individual culture vessels, (2) a fluidic module, which controls movement of liquid in and out for each culture vessel, and (3) a plug-and-play hardware infrastructure that simplifies high-volume bidirectional data flow by decoupling each parameter into individual microcontrollers (FIG. 4E). A detailed description of eVOLVER design and construction can be found in Example 10-18 (resources also available online at fynchbio.com).

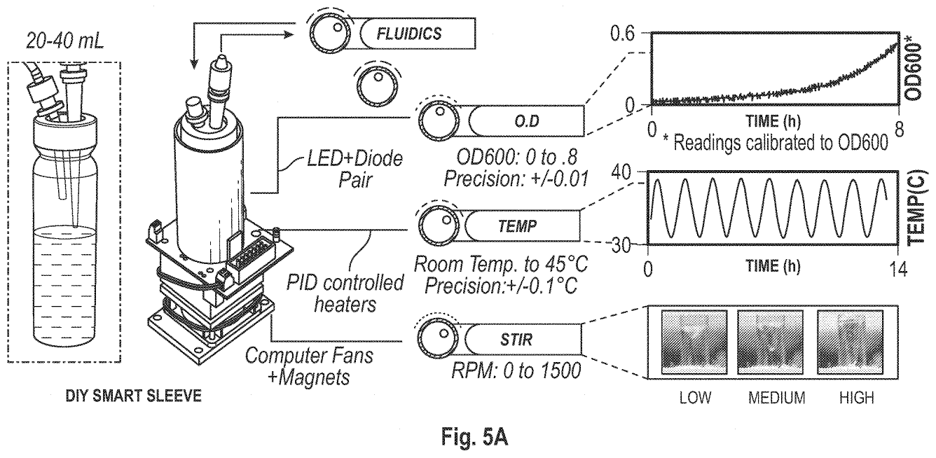

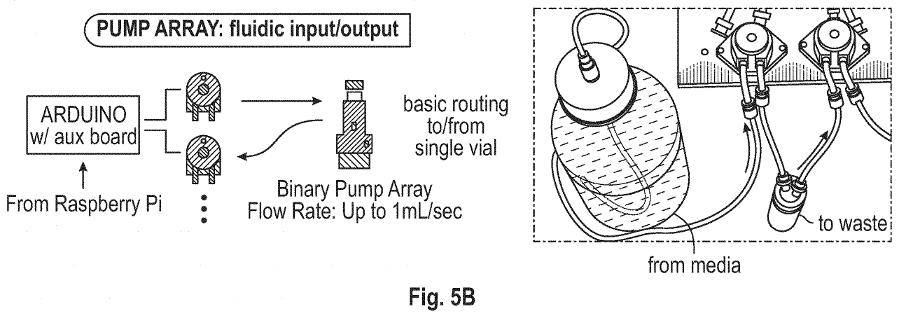

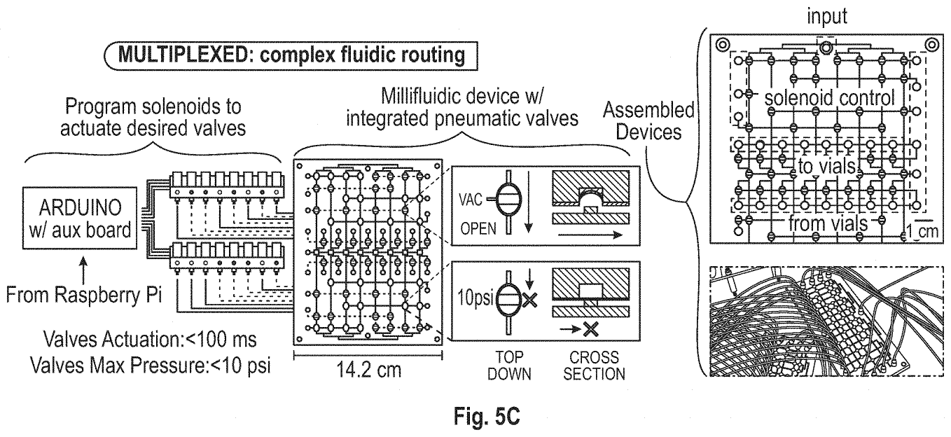

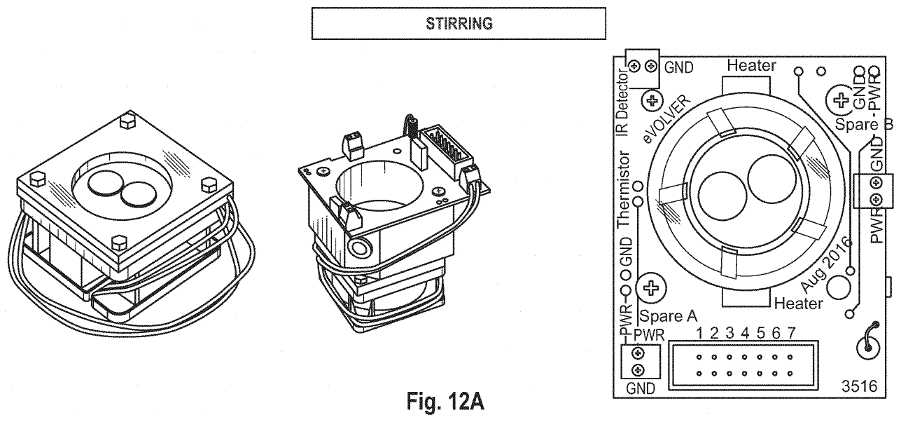

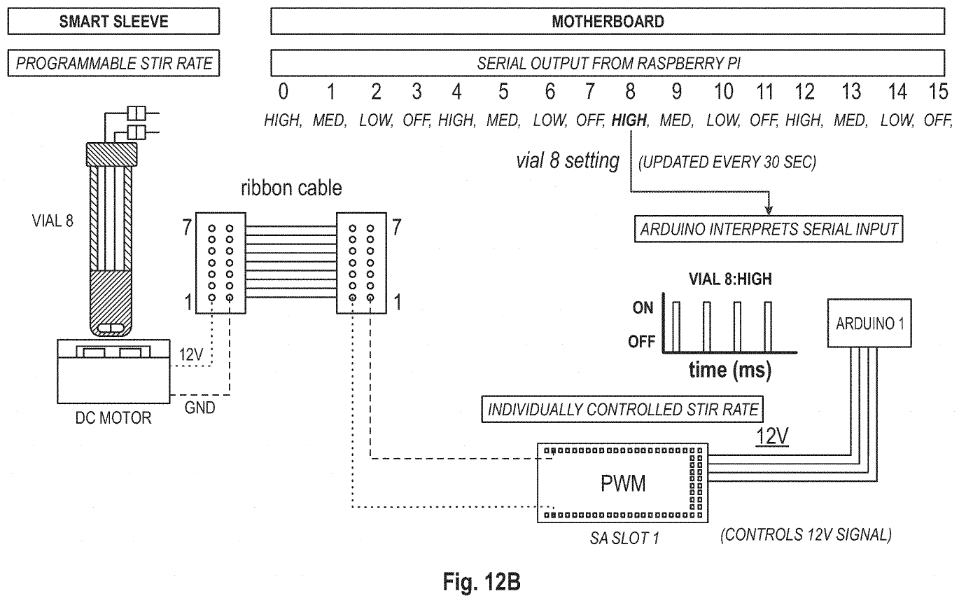

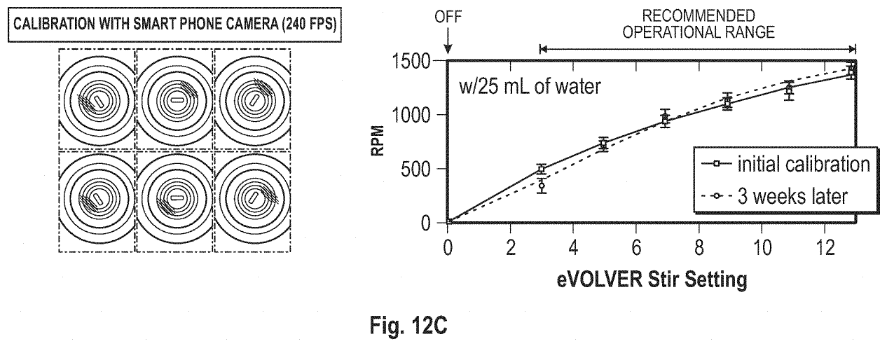

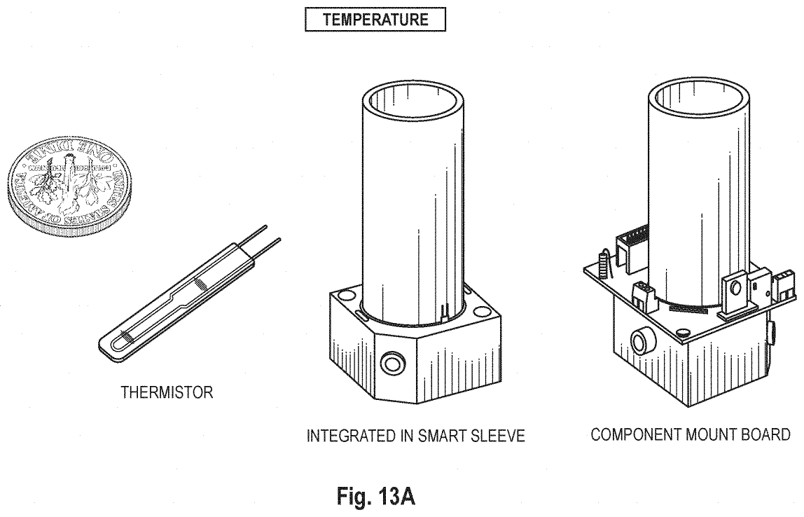

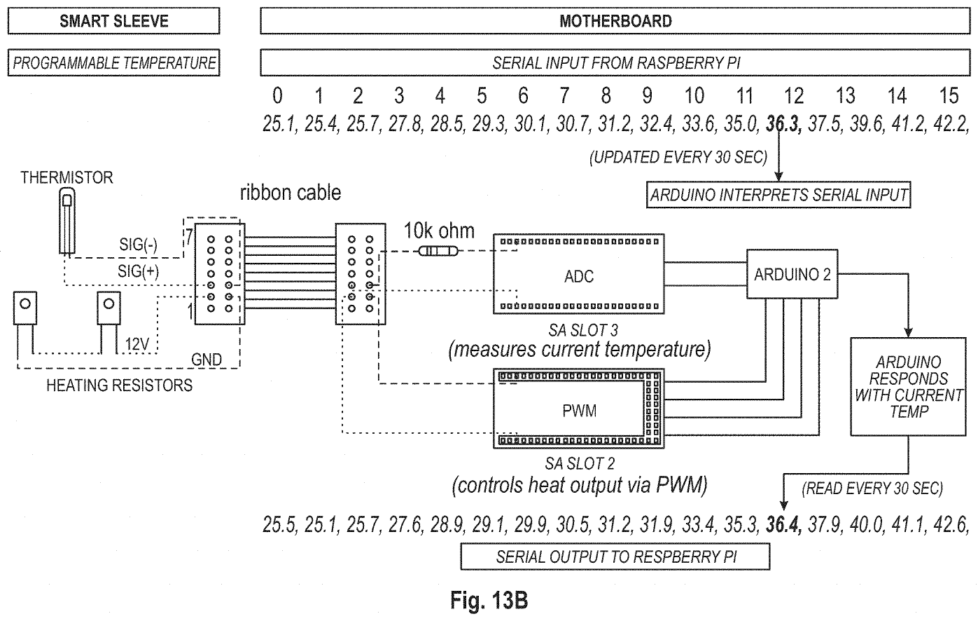

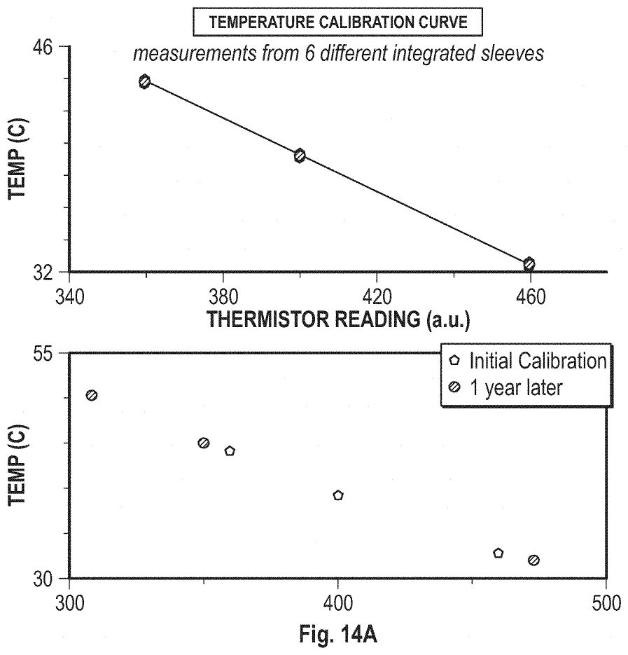

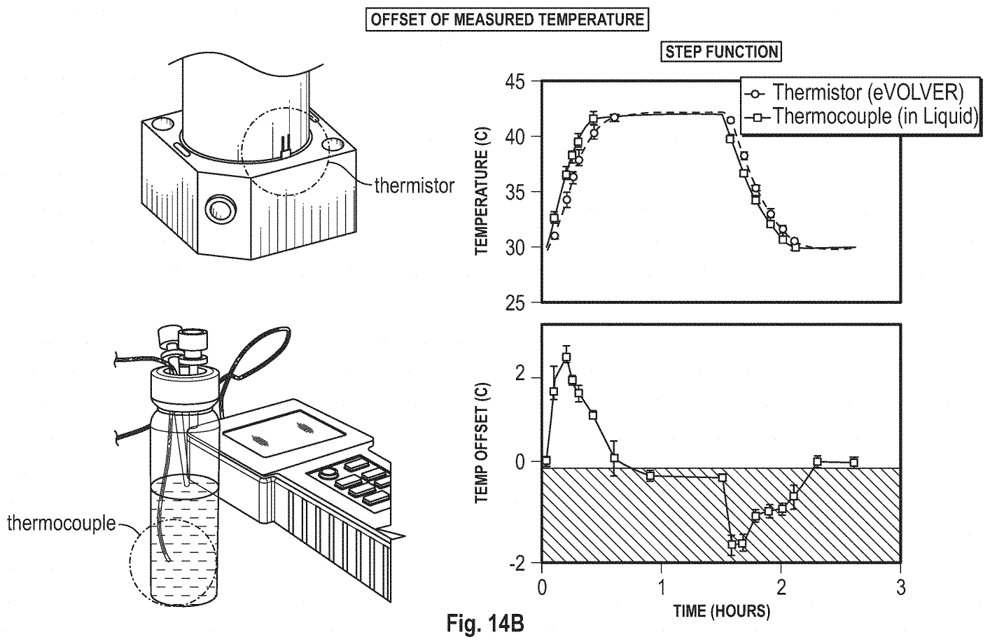

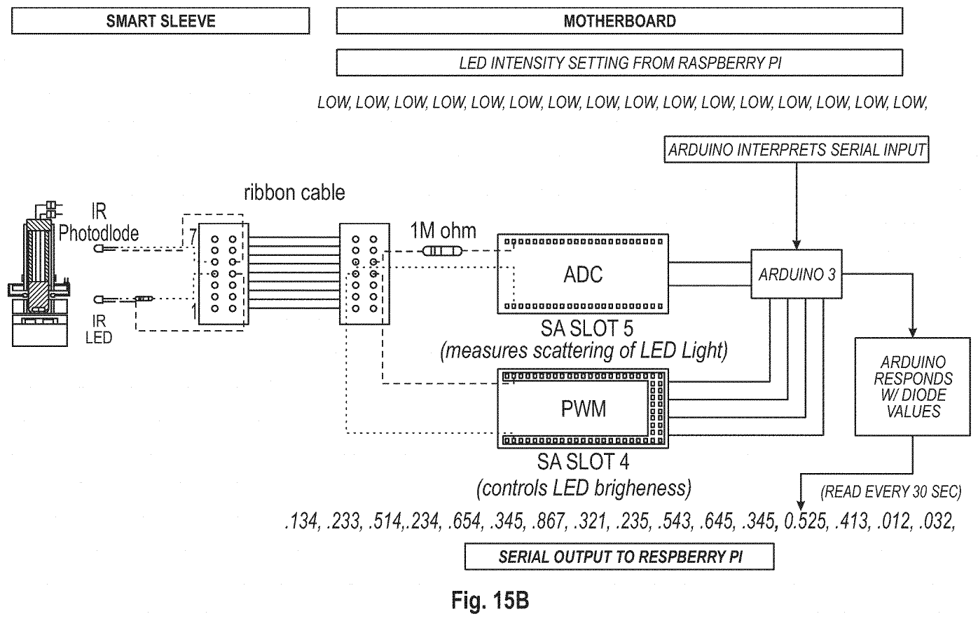

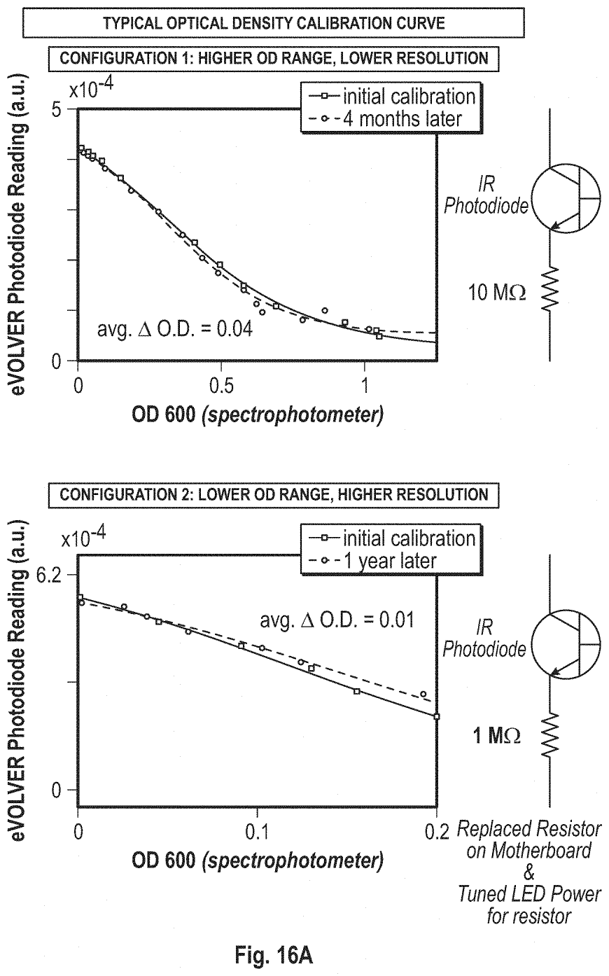

The Smart Sleeve is a manufacturable unit that mediates monitoring and control of growing cultures (FIG. 5A). Each sleeve is composed of a machined aluminum tube (for temperature control), printed circuit board (PCB) mounted sensors, actuators, and other electronic components, all attached to a custom 3D printed mount. As designed, the Smart Sleeve is one of the principal embodiments of eVOLVER versatility; it can be inexpensively mass-produced for high-throughput experiments, or reconfigured to meet custom experimental needs, such as larger culture volumes, pH or oxygen sensors for bioprocess applications, or light emitting diodes (LEDs) for optogenetic studies. In this work, we implement a particular Smart Sleeve configuration that accommodates 40 mL glass vials, and is configured to control three experimental parameters: stirring, temperature, and culture density (FIG. 5A, FIGS. 12A-12C, FIGS. 13A-13B. FIGS. 15A-15B).

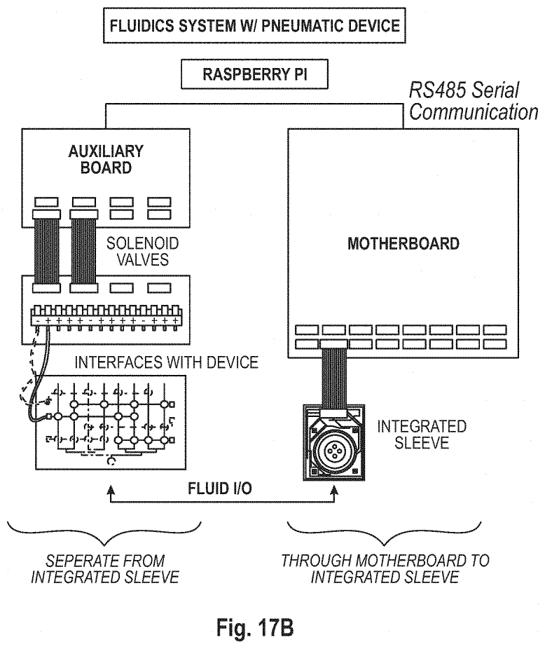

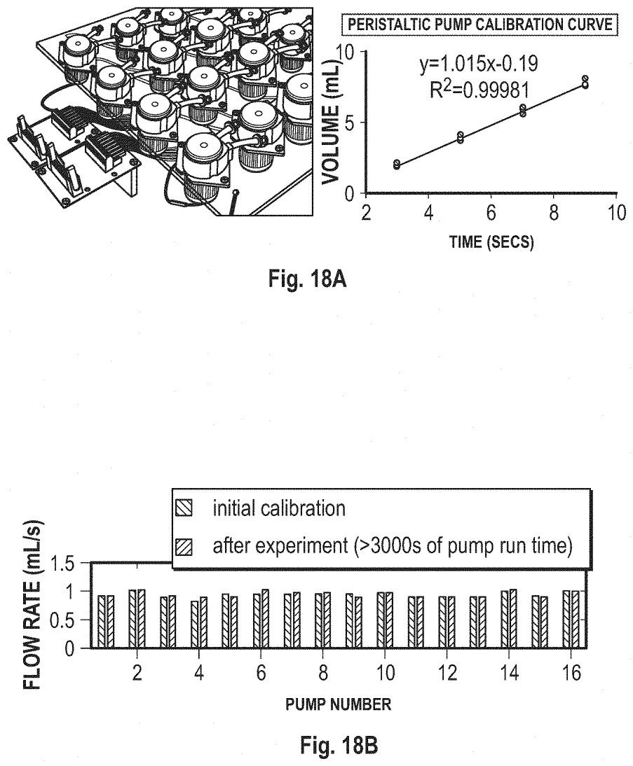

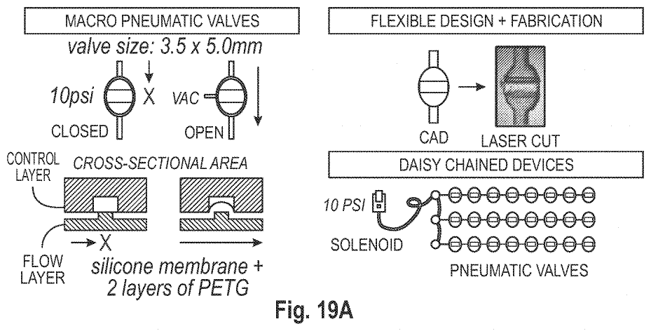

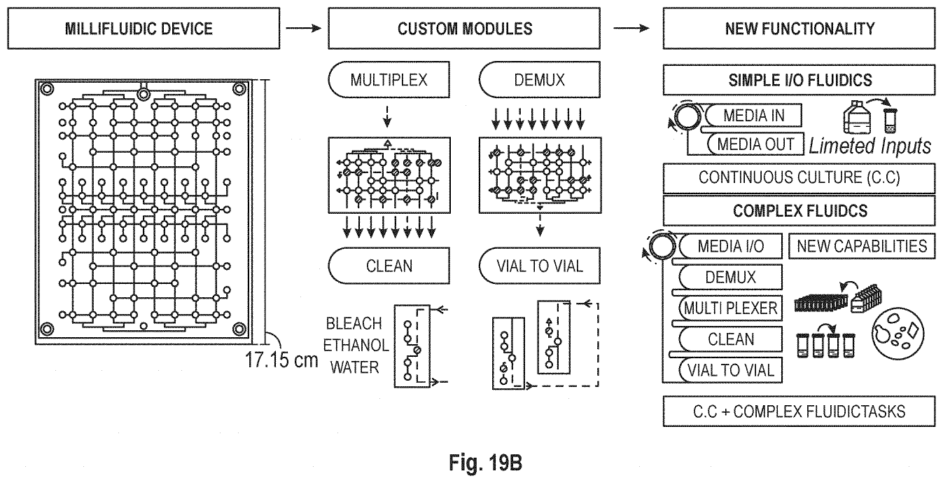

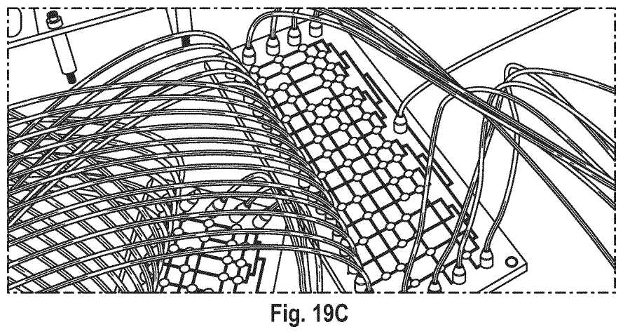

eVOLVER's fluidic module, which controls movement of media, culture, and liquid reagents within the system (FIGS. 5B-5C, FIGS. 17A-17B), can be configured to two fluidic handling modes: (1) “basic” mode, which uses peristaltic pumping to control media influx and efflux for each culture (Toprak E., et al., Nat. Protoc. 8, 555-567 (2013)) (FIG. 5B, FIG. 17B, FIGS. 18A-18B), and (2) a “complex” scheme in which multiplexed routing enables more sophisticated fluidic manipulations (FIG. 5C. and FIG. 17C). User-actuated peristaltic pumps are robust, simple to use, and can be scaled to carry out media dilution routines for a large number of parallel continuous cultures ( )Hope E. A., et al., Genetics 206, 1153-1167 (2017); Toprak E., et al., Nat. Protoc. 8, 555-567 (2013)) (FIGS. 18A-18B). However, they scale poorly to more sophisticated routing where the number of required connections and control elements expands non-linearly with the number of cultures. Our “complex” fluidic solution uses the principle of large-scale integration (LSI) to overcome this limitation. Originally developed for electronic devices, and then elegantly adopted in microfluidics (Thorsen T., et al., Science 298, 580-4 (2002); Melin J. and Quake S. R., Annu. Rev. Biophys. Biomol. Struct. 36, 213-231 (2007)), LSI uses combinatorial multiplexing to expand the number of input-output paths per control channel (Unger M. A., Science 288, 113-116 (2000); Grover W. H., et al., Sensors Actuators, B Chem. 89, 315-323 (2003)). Using LSI as inspiration, we created physically-compact millifluidic multiplexing devices by adhering a silicone rubber membrane between two clear sheets of laser-etched plastic, each patterned with desired channel geometries and aligned to form an intact device (FIG. 5C., FIGS. 19A-19C, FIGS. 34A-34C, FIGS. 35A-35C). Devices can be designed on-the-fly to carry out custom fluidic protocols, including complex media dispensing routines, transfer of liquid between cultures, or periodic cleaning protocols to maintain sterility. Full treatment of multiplex device design and fabrication can be found in Example 15, and a catalog of devices developed for this study can be found in the Example 6.

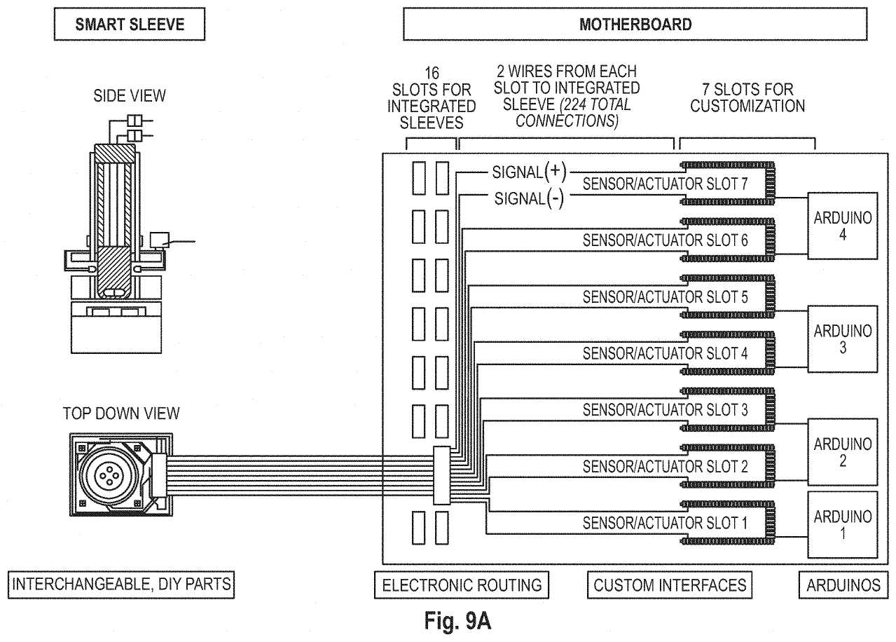

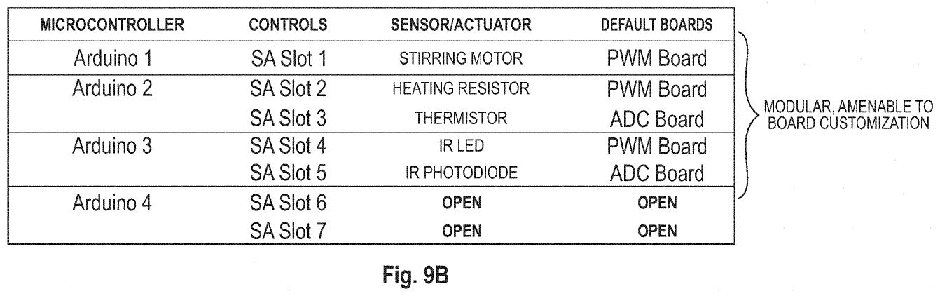

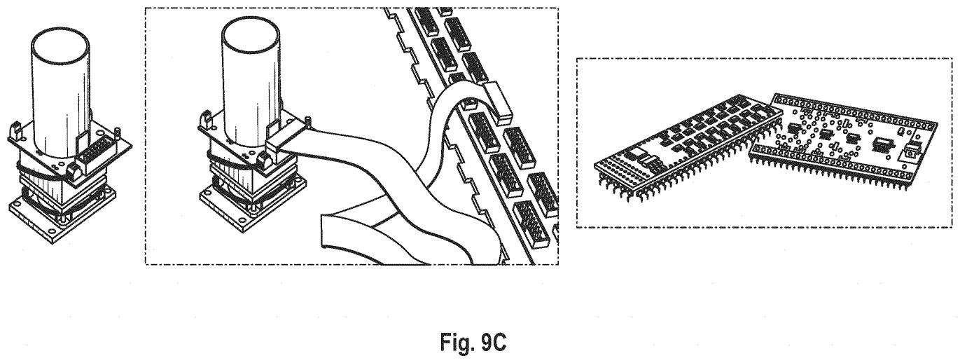

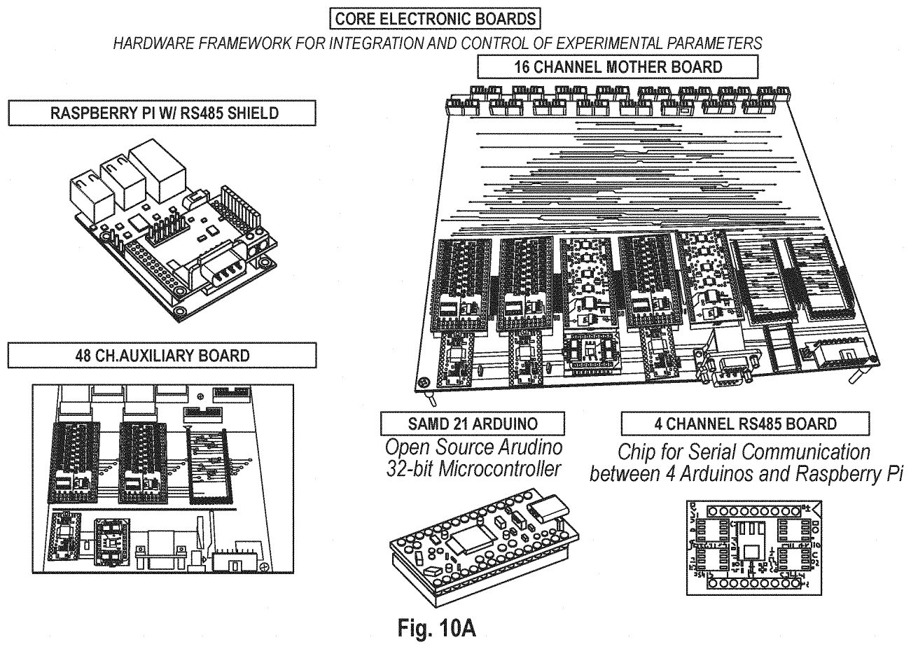

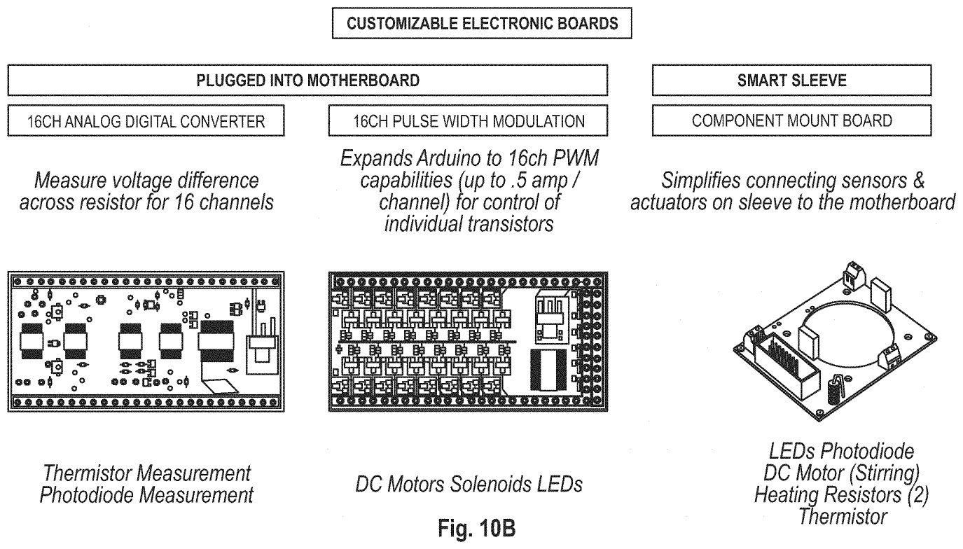

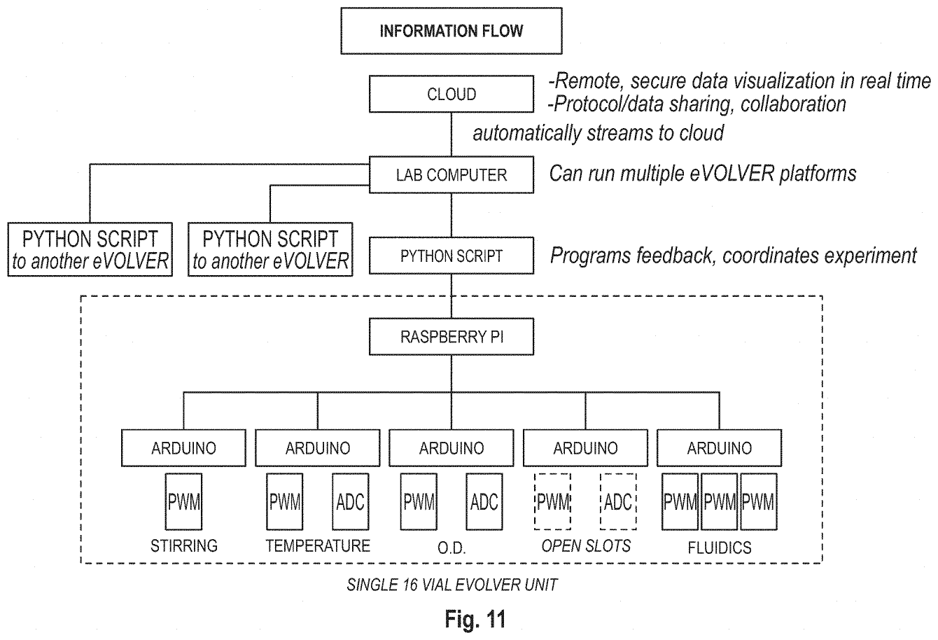

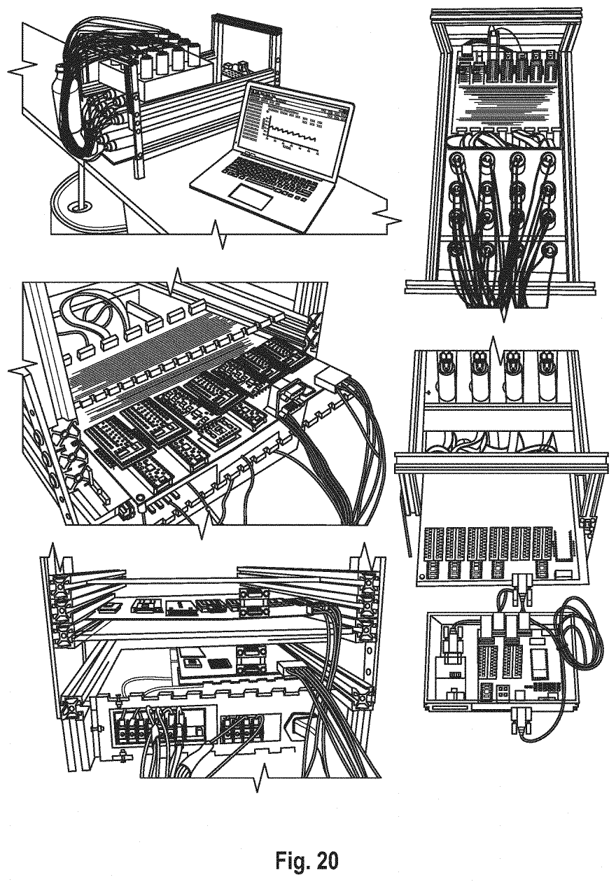

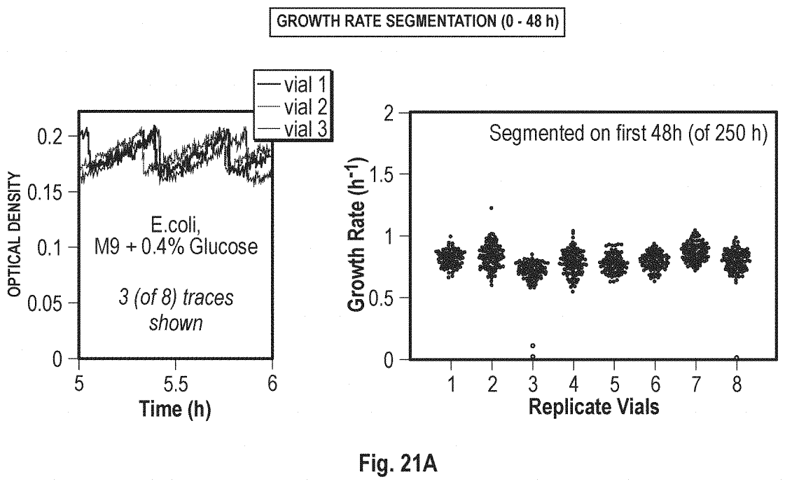

We developed a hardware and software infrastructure for eVOLVER that complements the scalability and configurability of the Smart Sleeve and fluidic modules (FIG. 4C). Our design is analogous to that of desktop computers, where a central organizing PCB (motherboard) is responsible for core functionalities (e.g. serial communication, signal routing), but also contains pluggable slots for boards that manage specialized features (e.g. graphics cards) (FIG. 4E). In eVOLVER, control modules—custom PCBs wired to Arduino microcontrollers-read and power Smart Sleeve-mounted components. During an experiment, individual control modules manage each culture parameter (e.g. temperature, stirring) (FIGS. 9A-9C and FIGS. 10A-10B). A motherboard distributes control of up to 4 independent parameters across a set of sixteen Smart Sleeves, comprising a single eVOLVER base unit (FIG. 4D and FIG. 20). Microcontrollers associated with each base unit are coordinated by a single Raspberry Pi, a small, low-cost, single-board computer that serves as a bidirectional relay to a user's computer or a cloud server (FIGS. 4C-4E and FIG. 11). The modularity of this design facilitates repurposing and scaling; users can easily modify or augment eVOLVER's experimental capability by connecting new control modules, while additional base-units can easily be cloned to achieve higher throughput (FIG. 4D and FIG. 20).

An important feature of eVOLVER's design is its ability to leverage network connectivity to coordinate and run experiments over the internet. eVOLVER's distributed hardware architecture enables efficient transmission of large packets of high-dimensional, real-time data (FIG. 4C and FIG. 4E) such that a single computer, located anywhere with an internet connection, can monitor hundreds of cultures in real time (FIG. 11). Python scripts running on the computer manage the acquired data and execute the control algorithms that define a selection scheme.

For example, during a typical data acquisition/control protocol, a scripted routine may query the Raspberry Pi every 30 seconds for Smart Sleeve-acquired culture status data (e.g. temperature, optical density, etc.) (FIG. 4C and FIG. 4E). After being recorded, higher-order data (e.g. growth rate) are computed by an algorithm and used to update individual Smart Sleeves or fluidic channels with new settings (e.g. adjusted temperature or media dilution). By modifying a script, the user can change selection criteria between different modes of automated culture (FIGS. 45A-45B), or specify a selection pressure surface through subtle iterations of the same algorithm across many Smart Sleeves.

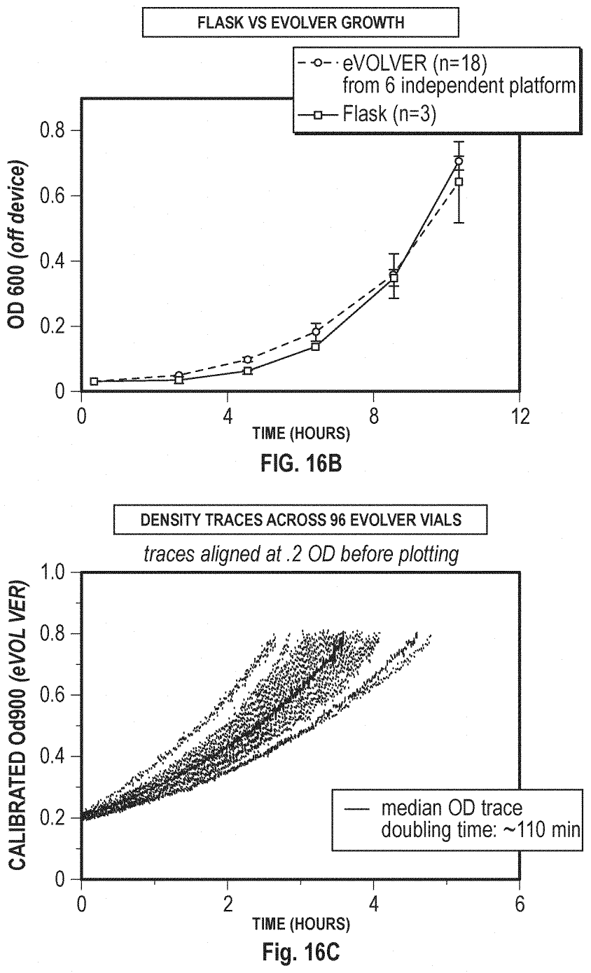

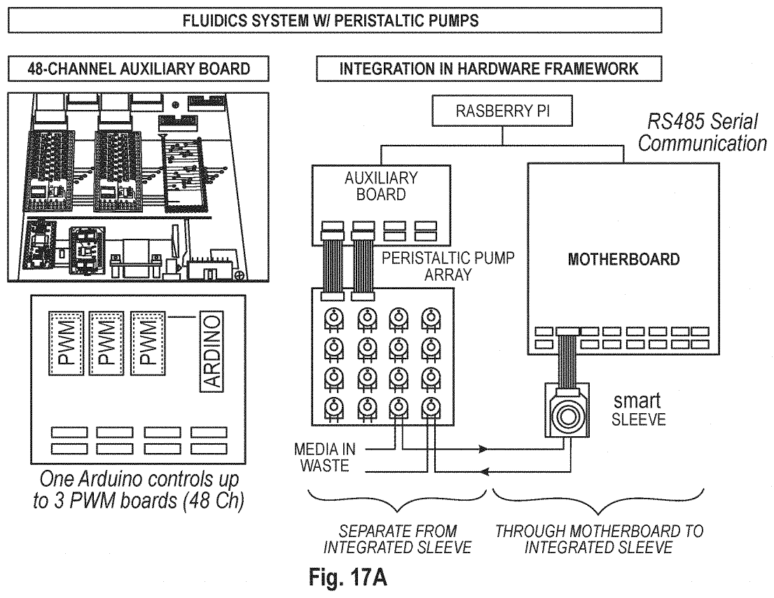

eVOLVER is capable of robust long-term operation. The system configuration described in this paper is capable of running long-term (250+h) experiments without electronics or software failure (FIGS. 6A-6C and FIGS. 21A-21B). Since most components have very long lifetime (>4000 h), hardware replacement is not required over the course of most experiments. Hardware calibration may be performed as frequently as desired, but we observed that settings remain essentially invariant over dozens of experiments (over 1700 h of operation) (FIGS. 12A-12C, FIGS. 14A-14C. FIGS. 16A-16C, FIGS. 18A-18B). eVOLVER is designed to be robust to catastrophic liquid spills, since Smart Sleeves and fluidic components are physically separated from their control hardware (FIG. 4E). Additionally, the system can be easily set up to avoid microbial contamination. In a control experiment, we found that the system remained sterile for the entirety of the experiment (˜10 days) while incubating uninoculated, antibiotic-free media alongside actively passaged E. coli cultures (FIGS. 21A-21B).

### Example 7. Conducting Experimental Evolution Across a Multidimensional Selection Space

In order to showcase eVOLVER's ability to conduct long-term continuous culture laboratory evolution experiments at high throughput, we explored the relationship between culture density and fitness in evolving yeast populations. To accomplish this, we configured eVOLVER to function as a turbidostat, maintaining culture density within a constant, defined window bounded by lower and upper OD thresholds (OD_{lower threshold}−OD_{upper threshold}). Using continuously recorded OD data (FIGS. 15A-15B and FIGS. 16A-16C), a routine activates dilution when the upper threshold is exceeded, and continues to fire until the lower threshold is reached (FIG. 6A). Population growth rates can be calculated in real-time by segmenting and fitting the OD trace between dilution events. We varied the upper and lower OD thresholds across 78 yeast populations grown in parallel, thereby defining a two-dimensional selection space based on minimum and maximum culture density (FIG. 6B) (see Example 6). Cultures were grown in glucose-limited media for 500 h (40-280 generations depending on the culture), resulting in diauxic shifts in cultures with OD windows that exceed ˜0.35 (FIG. 22). The frequency with which the culture experiences the shift is a function of density-dependent selection prescribed to individual cultures (FIG. 6B and FIG. 22). Recorded OD measurements allowed us to generate maps of population and evolution parameters across the selection space, including population growth rates (FIG. 23) and average genome replication events, a function of OD and growth rate (FIG. 6B and FIG. 24).

We then used eVOLVER to perform fitness measurements on isolates from each of the evolved populations by competing them against a fluorescently-labelled ancestor strain under low- and high-density continuous growth regimes (468 total cultures), assaying for population ratio over time using flow cytometry (Kryazhimskiy S., et al., Science 344, 1519-1522 (2014)) (see Example 6). The fitness distributions generated for low- and high-density regimes were distinct (FIG. 6C). K-means analysis of the fitness phenotypes yielded three distinct groups: low-density specialists, high-density specialists, and the remainder exhibiting low fitness in both measured niches (FIGS. 25A-25D). In general, low-density specialists evolved in niches with small lower OD thresholds. In contrast, high-density specialists were derived from cultures maintained in narrow, high-OD windows, rather than simply niches with high OD thresholds (FIG. 6C and FIGS. 25A-25D). Our results demonstrate that in applying multidimensional selection pressures, eVOLVER can be used to map pressures that drive adaptation to specific regions of the selection space.

### Example 8. Growth Selection Under Temporally Varying Selection Regimes

There is growing interest in understanding how an environment's temporal features shape organismal adaptation (Mitchell A., et al., Nature 460, 220-224 (2009)). A fluctuating environment may yield adaptations distinct from those selected under monotonic pressure (Ketola T., et al., Evolution (N. Y.) 67, 2936-2944 (2013); Sether B. E., and Engen S., Trends Ecol. Evol. 30, 273-281 (2015)). eVOLVER can be used to systematically vary temporal features of a selective pressure while holding other culture conditions constant. To demonstrate this, we performed growth selection experiments on a pooled YKO library (Giaever G., et al., Nature 418, 387-391 (2002); Li Z., et al., Nat. Biotechnol. 29, 361-7 (2011); Breslow D. K., et al., Nat. Methods 5, 711-718 (2008); Giaever G., and Nislow C., Genetics 197, 451-465 (2014)) (5,149 unique members) under conditions in which a single environmental variable—temperature—was temporally varied. A two-dimensional selection space was programmed by varying the magnitude and period of square wave temperature oscillations (FIG. 7A and FIGS. 14A-14C). Cultures were maintained for 6 days in turbidostat mode, and samples collected every 48 h (see Example 6). At the conclusion of the experiment, next-generation sequencing was performed on each selected population to determine library member frequency (Smith A. M. et al., Genome Res. 19, 1836-1842 (2009); Gibney P. A., et al., Proc. Natl. Acad. Sci. 110, E4393-E4402 (2013)), which was used to calculate the fitness of each member (Kryazhimskiy S., et al., Science 344, 1519-1522 (2014)) (see Example 6) (FIGS. 26A-26B, FIG. 27, and FIG. 28).

We computed a 2D weighted centroid for each library member by transforming its measured fitness into coordinates of temperature magnitude and frequency, allowing us to compare the library phenotypes across the 2D temporal selection space (see Example 6) (FIG. 7C). Plotting fitness centroids for the high-performing strains in each of the 16 conditions confirmed that the centroids cluster in the corresponding region of magnitude/frequency space (FIG. 29). Additionally, we picked four library members from different regions of the distribution (ΔHSP104, ΔKAP120, ΔAHA1, and ΔSWA2) for experimental validation of our library selection. We competed these strains against a neutral control strain (ΔHO) across four temperature profiles, observing fitness profiles that agree with the library selection results (FIG. 7D).

Library members with significant fitness centroid shifts along the magnitude or frequency axes were identified (FIGS. 30A-30B), including several chaperone and chaperone cofactor genes, which are known to play a role in thermal stress response (Morano K. A., et al., Genetics 190, 1157-95 (2012)). Saccharomyces Genome Database (SGD) (Cherry J. M., et al., Nucleic Acids Res. 40, D700-D705 (2012)) phenotype annotations were then used to identify sets of similarly annotated genes with fitness centroids significantly shifted from that of the population mean (FIG. 31). Next, by applying cross-correlation and principle component analysis to compare each condition, we observed three distinct groups of conditions with correlated effects on the library: two high-temperature groups corresponding to (1) high- and (2) low-frequency, and (3) a mild temperature group (FIG. 32 and FIGS. 33A-33C). We identified gene ontology (GO) term annotations that are linked to fitness defects in one or more of these groups (FIGS. 33A-33C). As expected, we observed that functions directly tied to growth rate (e.g. mitochondrial function, ribosome biogenesis) significantly altered fitness at mild temperature increases. Interestingly, ribosome components and processing factors also showed high-frequency sensitivity at high temperatures, suggesting a potential role for ribosome biogenesis in transitions in and out of stress. We further interrogated potential sources of frequency-dependence. We found that the high- and low-frequency groups were characterized by annotations associated with cell cycle checkpoints (e.g. DNA damage response, organelle fission), which temporally regulate cellular processes and thus might be expected to affect cellular response to fluctuating stresses at different frequencies.

### Example 9. Enabling Complex Automated Culture Routines with Fluidic Multiplexing Devices

In order to demonstrate the potential of the fluidic multiplexing framework to automate movement of reagents and cells in eVOLVER, we designed devices for three experimental applications: (1) dynamic media mixing during continuous culture to track yeast response to ratios of sugars (see Example 6); (2) preventing bacterial biofilm formation via automated passaging (see Example 6); and (3) programming sexual reproduction between adapting yeast populations (see Example 6).

We showed that fluidic multiplexing could be used to manage media composition for multiple cultures maintained by eVOLVER by constructing an 8-channel media selector device that dynamically draws media from multiple input sources and dispenses a defined mixture to a culture of choice (see Example 6) (FIG. 5C, FIGS. 34A-34C, and FIGS. 35A-35C). Galactose utilization in yeast is regulated by the ratiometric sensing of available galactose and glucose (Escalante-Chong R., et al., Proc. Natl. Acad. Sci. U.S.A 112, 1636-41 (2015)). We maintained a yeast strain harboring a galactose-inducible fluorescent reporter (pGAL1-mKate2) in continuous cultures featuring 16 different sugar compositions (FIGS. 36A-36C). Dyed media, tracking relative sugar levels in each culture, confirmed that the device can dynamically mix and dispense in correct ratios for extended periods of time (FIGS. 36A-36C). We used flow cytometry measurements to track galactose-induced population fractions, confirming maintenance of sugar-dependent gene induction (FIG. 8A).

The utility of the millifluidic system for mediating liquid transfer between cultures was demonstrated using a device that overcomes biofilm formation during long-term continuous growth experiments (FIGS. 35A-35C and FIG. 38). Serratia marcescens (Rice S. A., et al., J. Bacteriol. 187, 3477-3485 (2005)), a bacterial species that readily forms biofilm on glass surfaces, was grown in turbidostat mode. Using a vial-to-vial transfer device, inoculation of a fresh vial was performed at 8 h intervals, followed by automated sterilization of culture-exposed fluidic paths (FIG. 8B). While thick biofilm deposition was observed after only 14 h in a culture grown continuously in a single vial (no transfers), no deposition was observed in vials maintained under the transfer routine (FIG. 8B). With daily replacement of used vials, four Smart Sleeves could be used to passage a culture, biofilm-free, for an indefinite period of time (FIG. 39).

Finally, we demonstrated that multiple functionalities can be combined to automate complex fluidic handling routines. We designed a fluidic routine for automated sexual reproduction of adapting yeast populations by integrating multiplexed media selection, vial-to-vial transfer, and post-transfer device cleaning (see Example 6) (FIGS. 34A-35C, FIGS. 35A-35C, FIG. 38, and FIGS. 40A-40B). This routine was used to conduct mating between separate, opposite mating-type haploid yeast evolving under different antifungal selections: cyclohexamide (CHX) and ketoconazole (KETO) (FIG. 8C). Rather than manually sampling at arbitrary timepoints, once haploid cultures reached user-defined growth rate milestones, automatic sampling and mating were carried out on the device (FIGS. 40A-40B and FIGS. 41A-41D). A minimum inhibitory concentration (MIC) assay performed on both diploid and parental populations indicated that the haploid-evolved CHX resistance was transferred to diploids in a dominant manner (FIG. 8C and FIG. 42). Conversely, the general antifungal resistance that emerged under KETO selection appeared to be recessive. Further sequencing of KETO-evolved haploid strains revealed nonsense mutations in ERG3 (FIG. 44), which has been shown to confer recessive resistance to azole antifungals (Anderson J. B., et al., Genetics 168, 1915-23 (2004)).

### Example 10. eVOLVER Design and Construction

Here we present the eVOLVER platform, a multi-objective framework for diverse automated cell growth applications. The eVOLVER platform is designed for high-throughput real-time monitoring and continuous control over experimental parameters for large numbers of cultures in parallel. We define experimental parameters as the measurable and/or controllable aspects of the culture environment: for the experiments in this study, we focus on stir rate, temperature, optical density, and rate/composition of fluid flow, but these could be expanded to include any number of parameters (e.g. culture pH, other optical probes). For multiple experimental parameters, how is individual control over many culture vessels achieved? First, the key to individual control lies in equipping each culture vessel with a programmable “Smart Sleeve”, which contains the hardware required to measure and adjust each experimental parameter. Activity is customized and coordinated across Smart Sleeves in a robust, open-source hardware infrastructure capable of monitoring and regulating each parameter in real-time, outlined in Example 11. Second, developing eVOLVER as a network-based framework proves to be enabling in two ways (Example 12): (a) network connectivity/compatibility enables a single computer to efficiently communicate to and operate several devices in parallel, creating a distributed solution for scaling to large numbers of cultures: (b) implementing modern communication protocols to stream data unlocks the internet of things, empowering users with the ability to remotely monitor an experiment and compile data in cloud-based databases. In Example 13, we describe how to calibrate and control the core experimental parameters for the Smart Sleeve configuration employed in this study, as well as introduce the methodology for customizing (modifying, adding or subtracting) experimental parameters according to the needs of the user. In Example 14, we introduce the fluidic module of eVOLVER, which similarly to the Smart Sleeves, can control different configurations of liquid handling elements, such as an array of peristaltic pumps, to control fluid flow individually for each culture vessel simultaneously. Next, in Example 15, we describe a paradigm for programmable fluidic handling in continuous culture, achieved by novel millifluidic devices with integrated valves. Leveraging the eVOLVER fluidic module to control these devices, we vastly expand the capabilities of the system beyond simple input/output functions to complex fluidic manipulations for new continuous culture applications. In Example 16, we discuss specific strategies for implementing additional commonly sought functionalities in the eVOLVER framework. In Example 17, we address considerations for use of eVOLVER, including media requirements, network connectivity, device maintenance and lifetime, and prevention of contamination.

### Example 11. Open-Source Hardware Framework

General Summary:

The eVOLVER hardware framework contains three levels of organization: (1) programmable sensors and actuators (e.g. Smart Sleeve components, pumps/fluidic control elements) (2) a Motherboard and microcontrollers, and (3) a Raspberry Pi. At the level of individual culture vessels, Smart Sleeves enable individual control over several experimental parameters in the culture (FIGS. 5A-5C and FIGS. 9A-9C). Specifically, each sleeve contains sensors and actuators (e.g. heaters, LEDs, thermometer/thermistor) that measure and adjust aspects of the culture environment of a glass vial housed within. At the next level of organization, the Motherboard, Arduino microcontrollers, and other core electronic boards form a robust hardware infrastructure that communicates internally and coordinates activity of each individual Smart Sleeve to control each experimental parameter. At the final level of organization, a Raspberry Pi forms a link to the outside world by relaying information and commands to and from a computer/server, permitting the same computer/server to run many eVOLVER devices across a network. Layered on top of the hardware framework, control software enables programmable feedback between parameters and orchestrates experiments at an abstract level, providing an easy method of customization that is shareable with other users. Below we present the core hardware framework as well as the particular configuration enabling the experiments described in this study.

Smart Sleeve:

The programmable Smart Sleeve is the foundational unit on which the eVOLVER is built (FIGS. 5A-5C and FIGS. 9A-9C). The Smart Sleeve is comprised of all the sensors and actuators required to control the culture conditions inside a 40 mL borosilicate glass vial. At the core is an aluminum sleeve, which surrounds the vial and is used to control temperature via two resistive heaters and a thermistor integrated within. Near the base of the vial sits a 3D printed part that houses and aligns the optical density LED and photodiode. Below that sits a fan motor equipped with magnets to rotate a stir bar within the vial. The Smart Sleeve represents one of the most easily customized features of the eVOLVER: by changing which sensors and actuators are used and their layout, the user may develop culture vessels that fit their experimental needs. For a detailed description of the sensors and actuators used to control stirring, temperature, and optical density in Smart Sleeves featured in this study, as well as strategies for modifying the Smart Sleeve to fit experimental needs, refer to Example 13. Liquid handling is also controlled at the level of the individual culture vessel, yet these components are housed in a separate fluidic module, described in Example 14.

The sensors and actuators on each sleeve are integrated in a small printed circuit board (PCB), termed the Component Mount Board (CMB). We designed the CMB such that we can easily solder electrical connections and efficiently manage/package wiring from the sensors. The CMB is a very simple PCB, containing only a few resistors, and is straightforward to redesign and inexpensive to manufacture, if needed. The simplicity in the CMB leads to robustness in the system. For example, any accidental overflow and spillage from the vials (e.g. from clogged fluid lines or user error) should minimally impact the rest of the system, as critical components are located at the Motherboard rather than the sleeve itself. Ribbon cables provide a modular way to connect the integrated Smart Sleeves to the Motherboard.

The CMB is designed to rest atop a 3D printed piece, which houses optical density and temperature components (see Example 13). The printed part can be fabricated with any commercial or DIY 3D printer, readily available at almost any university or hacker space, and customized to the requirements of the user. For example, if a user wanted to change the mode of optical density detection between scattering and absorption, they could redesign the 3D printed part housing the LED-diode pair such that it would have the correct offset angle for the desired mode of measurement.

Motherboard:

Forming the core of the hardware framework, the Motherboard is designed to be modular and enable individual control of an array of Smart Sleeves. In particular, PCBs can be designed to plug into the Motherboard for customization of how sensors are read or actuators are controlled. In the depicted setup, the Motherboard contains 7 customizable sensor/actuator slots (SA slots) that interface with the components of the CMB for each Smart Sleeve (FIGS. 9A-9C). In this study, we used 5 of the 7 slots for control of three experimental parameters (stir, OD, temperature). The two additional slots can be used for custom sensors or actuators to expand capabilities with new parameters. Alternatively, one can retool any of the other 5 SA slots for their own experimental needs.

In more detail, two wires from each of the sensors/actuators on the CMB, bundled in a ribbon cable, are electrically routed through the Motherboard to one of 7 different SA slots. A total of 224 wires (7 SA slots×16 vials×2 wires) is required to properly route all sensors/actuators to the correct SA slots. Each SA slot consists of an array of 70 metal female pins. A PCB with the correct male pin layout would be able to plug into a slot, namely the customizable control boards (FIGS. 9A-9C and FIGS. 10A-10B). These are modular PCBs that can either read a sensor or power an actuator, permitting measurement of or control over parameters, respectively. In the configuration described in this study, we engineered two boards to occupy these slots, the ADC board and the PWM board, which are described in more detail below. Technical information on all boards is available online at fynchbio.com.

Arduino Microcontrollers:

The 7 customizable SA slots are organized under 4 SAMD21 Arduino Mini microcontrollers. This layout permits control over 4 different experimental parameters. Experimental parameters are controllable characteristics of the culture, such as the temperature or stir rate. The control of one parameter often requires more than one sensor and/or actuator, and thus requires more than one SA slot. For example, to control the temperature (parameter) of the culture, one SA slot is used to measure temperature (sensor, interfaces with ADC board) and another is used to heat the culture (actuator, interfaces with PWM board). The boards at these two SA slots are controlled by a single microcontroller to efficiently coordinate SA activity (e.g. sequential tasks, fast feedback control).

Managing several experimental parameters simultaneously across several cultures is a non-trivial, data-intensive task. The use of multiple microcontrollers permitted us to divide the load by experimental parameter for functional parallelization. Our design is analogous to that of personal computers, where specialized functionalities are enabled by modular supplementary electronics (e.g. graphics cards) which interface on a single motherboard. This approach was crucial for managing the complexity inherent in defining multidimensional growth environments.

Importantly, this design also facilitates modifications to the system by segregating each function, analogous to reconfiguring or swapping out components in a desktop computer. When modifications are made (either to microcontroller software or to the boards interfacing at the SA slot), adjustments are confined within a single experimental parameter and do not impact any other parameters of the system.

16-Channel Pulse Width Modulation (PWM) Board:

One of the customizable control boards, the PWM board is designed to plug into the Motherboard and enable an Arduino to easily and quickly control many actuators (e.g. motors, LED, heaters) in parallel. For example, 16 individual LEDs can be connected to the PWM board and each of the LEDs can be set to a different brightness and updated to a different value in fractions of a second. The board has two main functions: (1) amplifying the 3.3V signal from the Arduino to a higher output voltage (5V to 24V), depending on the voltage source, and (2) expanding the Arduino pulse width modulation (PWM) capabilities from 3 to 16 channels. PWM is essential since it allows digital signals to have a more analog-like output. Analog-like outputs enable finer control of experimental parameters. For example, the temperature control of the system would be noisier if the input was toggling between the heaters fully on and off. With PWM, the user can instead use a simple PID controller to feedback from temperature measurements to optimize for a specific, highly controllable heat output. The PWM board can be daisy chained such that a single Arduino can in principle control hundreds of channels.

16-Channel Analog Digital Converter (ADC) Board:

Another customizable control board, the ADC board is designed to plug into the Motherboard and measure the signal from dozens of sensors in the system. The sensors currently integrated in each sleeve are simple and can be measured with basic voltage divider circuits. The sensor and resistor are placed in series, and the voltage across a resistor changes when the measurement from the sensor changes. The board reads this voltage and has two main roles: (1) remove noise from the signal through a low pass filter and (2) multiplex the signal from all 16 channels to one analog input pin on the Arduino. When the signal arrives at the input pin, the Arduino changes the analog signal to a digital signal via its own 12-bit ADC.

### Example 12. Distributed Network Architecture

Communication and Parallelization Via Raspberry Pi:

Most laboratory equipment is controlled via local serial communication. Likewise, in eVOLVER, a RS485 serial communication protocol is used internally to send updated parameter values and receive current measurements to/from the Arduinos. All serial communication occurs on the same channel. In typical lab equipment, the data is then transmitted to a local computer via USB, usually requiring the equipment and computer to be in the same physical location. This may work well for a single system, but physical limitations arise when trying to scale to high-throughput studies on multiple systems (e.g. 100's of vials). Consequently, a solution was needed to scale the interface between many eVOLVER units and a computer/server monitoring the experiment.

To address this, eVOLVER is designed as a network-based tool, operating similar to how servers and computers communicate within the same network at a university or company. Each 16-vial eVOLVER unit contains one Raspberry Pi, a small Linux board, that helps relay information from the device back to the computer via an Ethernet port. Performing three main functions, the Raspberry Pi board: (1) enables the system to easily interface with modern internet protocols, (2) monitors and updates the Arduino microcontrollers with the desired configuration settings (e.g. temperature set points, fluid commands), and (3) gathers data from the Arduinos for user consumption. This enables a single laboratory computer/server to run many eVOLVER units distributed across physically different locations (e.g. different rooms, floors), since the devices can be connected via router (FIG. 11).

Directing Customizable, Repeatable, and Shareable Experiments with Software:

Each time a user runs a unique eVOLVER experiment, a new custom Python control script is generated. This has two major implications for running continuous culture experiments. First, experiments are easily customizable by simply adapting the Python code to meet the needs of the experiment without changing any of the hardware components. This approach is used throughout this study, for everything from defining different experimental conditions (e.g. optical density windows, dynamic temperature profiles), to feedback between parameters (e.g. turbidostat dilution calculations), to incorporating higher-level computations (e.g. growth-rate dependent event triggering). Second, experiments are easily replicated by simply copying the Python script from one experiment to a new file. By using the exact same control algorithms, the conditions can be tightly matched between experiments. This approach is used throughout this study, particularly for validation experiments, in which a strain isolated from an evolution or genetic screening experiment is then tested in the same environmental conditions from which it was isolated. Furthermore, this permits sharing protocols between users who have the same hardware layouts. Not only is this advantageous for collaborations between labs and replication studies, but it also accelerates the rate at which new users can learn the device. Basic experiment control scripts are made available at fynchbio.com; online code repositories may be used to collect and share additional, user-generated experiment control scripts.

Connecting Biological Laboratory Equipment to the Internet of Things:

Typically, in the laboratory, experimental data is collected, analyzed, and stored in local files on a user's computer. Each user has their own preference or standard procedure to analyze and display the data, making sharing and curating information difficult. Consequently, though potentially valuable, raw data is infrequently shared. The eVOLVER framework offers a solution to this problem. Since eVOLVER uses modem communications protocols, the device can stream data directly to a database and utilize cloud tools. This can facilitate how experiments are done in several ways: (1) real time monitoring of experiments from anywhere with an internet connection. (2) standardization and curation of growth data between experiments, and (3) interfacing with cutting-edge cloud tools for analysis and segmentation of data. These aspects of the eVOLVER framework promote scalability even beyond high-throughput experiments, facilitating modern ways of ingesting and analyzing data. Towards this end, we have developed back-end code and a Python library to easily stream data from eVOLVER to a server for real time visualization and analysis of data. More information can be found at fynchbio.com.

### Example 13. Distributed Network Architecture

Customizability was a key design consideration when developing the eVOLVER. In Example 12, we describe the utility and ease of writing software to program feedback between experimental parameters for designing novel experiments. In this section, we describe how one can customize the hardware to modify/add parameters of interest. Additionally, we present the components and systems used for the measurement and control of three core experimental parameters in each Smart Sleeve: stirring, temperature, and optical density.

Modifying, Adding, and Removing Experimental Parameters:

A key feature of our hardware framework is that it enables adding, subtracting, and modifying components without changing the rest of the system (FIGS. 10A-10B). For example, if the user wants to add an LED to each culture vessel for dynamic light induction during continuous culture, traditionally this would require redesigning and rebuilding the entire system. In contrast, using the eVOLVER framework, the user can add experimental parameters with minimal modifications to the current system. For example, to add light induction as an experimental parameter, the user could follow these steps:
1. Modify the Smart Sleeve to incorporate the LED where desired on the device. This involves only redesign of the CMB, the 3D printed tube holder, and the aluminum sleeve. These components are simple to design and fabricate with minimal experience. Template designs are made available at fynchbio.com.
2. Plug in PCB to corresponding SA slot. The LED must be properly connected to the ribbon cable via the CMB, making an electrical connection to one of the 7 SA slots. To control an LED, a PWM board (same as above) can be used to control the intensity. Alternatively, if the user has additional specific requirements, one could engineer their own custom control PCB to plug into this slot to control the LEDs.
3. Program the Arduino micro-controller to customize how serial commands translate to LED brightness. First, set up a unique address of the Arduino micro-controller such that the serial commands matching this address will be interpreted by the correct Arduino. Second, custom routines can be programmed to permit rapid computation, sequential actions, or internal feedback. Template routines are made available at fynchbio.com.

Specifics for implementing additional commonly desired parameters and functionalities in eVOLVER are found in Example 16.

Stirring:

The eVOLVER platform features tunable and independent stir rate control across culture vials. Stirring in eVOLVER is actuated by 12V brushless DC motors with attached neodymium magnets. The fastened magnets spin a stir bar (20 mm×3 mm, PTFE coated) within an autoclaved glass vial (28 mm×95 mm, borosilicate). The stirring module utilizes a single SA slot on the Motherboard; in the particular configuration described in this study, we utilized SA slot 1 (FIGS. 12A-12C). The two leads of the motor (12V & GND) are connected to a screw terminal on the component mount board, from which a ribbon cable connects the smart sleeve to the Motherboard. The PWM board (plugged into the SA slot) can control each motor independently to achieve different stir rates across eVOLVER vials. Briefly, the 16-channel PWM board amplifies a 3.3V signal from the Arduino microcontroller to a 12V signal to actuate the motor. Arduino 1, which manages SA slot 1, was programmed to take in serial inputs from the Raspberry Pi and translate the serial values to different stir rates, determined by pulsing the motor ON and OFF at different ratios (FIGS. 12A-12C).

Temperature:

In contrast to current approaches in which all culture vessels are housed in a single incubator (Toprak E., et al., Nat. Protoc. 8, 555-567 (2013); Takahashi C. N., et al., ACS Synth. Biol. 4, 32-38 (2015)), we developed a module for individually controlling the temperature of each Smart Sleeve in the eVOLVER. This not only allows the cultures to be maintained at distinct temperatures, but also reduces thermal mass, permitting dynamic temperature profiles. For the configuration described in this study, the temperature module utilizes SA slots 2 and 3 on the Motherboard (FIGS. 13A-13B).

Typically, there are three main components to temperature control: (1) a thermometer, (2) a heater, and (3) a feedback controller. In our setup, the thermometer and the heater are integrated in the Smart Sleeve while the feedback controller is located on the Motherboard. Specifically, the temperature is measured by a 500 μm thick temperature-sensitive resistor, or thermistor (Semitec, 103JT-025). The sensor is integrated into the sleeve between the 3D printed part and the aluminum tube, and the thermistor is soldered onto the component mount board (CMB) after assembly. The aluminum tube enables even heat distribution/dissipation and shields the culture from ambient light (important for other measurements/parameters). Two heating resistors (20 Ohm 15 W, thick film) are screwed onto the aluminum tube for better contact and connected to the CMB via soldering. In our setup, the four leads, 2 from heating resistors and 2 from thermistor, are connected via a ribbon cable to the Motherboard and routed to SA slots 2 and 3, respectively. In slot 2, a 16-channel PWM board amplifies a 3.3V signal from the Arduino microcontroller to a 12V signal to actuate the heating resistors. Slot 3 contains a 16-channel ADC board, which reads the voltage difference across a 10 kilo Ohm resistor, and is responsible for analog filtering and demultiplexing the signal from the thermistor. These slots are connected to and are programmatically controlled by Arduino. Briefly, the Arduino code interprets serial inputs from the Raspberry Pi, updates the set point on the PID controller, and responds with the current measured temperature. Temperature settings can be updated as frequently as every 30 seconds. To determine how much to turn the resistive heaters on, the Arduino is programmed with a simple PID control algorithm. The PID controller can be easily tuned via software to obtain the desired overshoot and time delays. The Arduino then controls a PWM board (on SA slot 3) to interface with the resistive heaters and get the desired heat output.

Calibration of the temperature measurement in the sleeve was performed by comparing the temperature of water measured in the vial using a thermocouple to the values returned by the thermistor (FIGS. 14A-14C). The dynamics of heating were determined by tracking temperature during a programmed step function, again comparing thermocouple and thermistor readings; the thermistor measures the temperature of the sleeve, while the thermocouple measured the actual water temperature. At room temperature (23° C. for this experiment), a single culture (20 mL) can reach a temperature of 42° C. in roughly half an hour with the current hardware setup (FIGS. 14A-14C). During an experiment, the transient offset between the recorded temperature and actual temperature may vary due to ambient temperature and volume of liquid. At steady state, the temperature can be maintained to +/−0.1° C., with properly tuned PID constants. Max temperature and rate of temperature ramp can be changed with different power sources (e.g. 24V power source could reach temperatures >55° C.).

It should also be noted that at different temperatures, the optical density readings are affected accordingly. This effect was measured in both yeast cultures and evaporated milk (FIGS. 14A-14C). See the next section for more detail.

Optical Density:

Based on previous work (Toprak E., et al., Nat. Protoc. 8, 555-567 (2013)), optical density measurements in a bioreactor can be measured with a simple 900 nm infrared (IR) LED and photodiode pair. There are two practical benefits of using 900 nm scattered light instead of the classic OD₆₀₀. First, at 900 nm, turbidity/optical density measurements are less dependent on the absorbance spectrum of the media, meaning calibration is required less frequently before each experiment. Second, wavelengths in the visible range are preserved for light induction and colorimetric assays. To maximize scattering, the LED-diode pair is offset at a 135° angle. The 3D printed part is designed to house the LED-diode pair slightly above the height of the stir bar, at the correct angular offset. The part can be easily customized and printed to the users required specifications with any 3D printer.

In the eVOLVER configuration used in this study, the IR LED and photodiode pair (4 leads) are each connected to the CMB via screw terminals in SA slots 4 and 5, respectively (FIGS. 15A-15B). In SA slot 4, a 16-channel PWM board amplifies a 3.3V signal from the Arduino microcontroller to a 5V signal to power the IR LED. A resistor is placed on the CMB to limit current and prevent the LED from burning out. SA slot 5 contains the 16-channel ADC board, responsible for analog filtering and demultiplexing the signal from the photodiodes. The ADC board reads the sensor by measuring the voltage difference across a 1 M Ohm resistor, located on the Motherboard. Both slots are managed by Arduino 3 in the system developed in this manuscript. Briefly, the Arduino code interprets serial inputs from the Raspberry Pi, flashes ON the IR LEDs to measure turbidity, and responds with the current measurements. In the present system, optical density can be measured every 30 seconds, limited by the time taken for the Arduino to average diode readings (to minimize noise).

For convenience, density readings from the 900 nm LED-diode pair were calibrated to OD₆₀₀ measurements from a spectrophotometer, and the calibration curve fit with a sigmoidal function (FIG. 16A). Spectrophotometer readings were performed on a Spectramax M5 using 300 uL of media in a 96-well flat bottom plate; users may substitute density calibration data from measurements used in their labs. The optical density measurements in all experiments are calculated based on the calibration curve fit for each Smart Sleeve (FIGS. 16A-16C). For our experiments, calibration was performed using a dilution series of yeast cells suspended in distilled water, but in theory any cell type and/or solution of interest (such as evaporated milk) could be used. A custom MATLAB script was developed to facilitate the density calibration process, particularly important for bringing new eVOLVER units on line. Following calibration, the system was used to compare growth of S. cerevisiae (FL100) cells in eVOLVER vials to that in 250 mL flasks with 50 mL of media shaken at 300 rpm (FIGS. 16A-16C). Finally, to quantify the variance in growth across eVOLVER vials, 96 cultures across six 16-vial eVOLVER units were grown in parallel and aligned (FIGS. 16A-16C). These results demonstrate that eVOLVER cultures are repeatable, and exhibit comparable growth rates to cultures in shaken flasks.

As previously mentioned, varying temperature induces a shift in the optical density readings (FIGS. 14A-14C). In measurements performed on yeast cells, we observed the largest shift near the center of the optical density calibration curve, while at low or high OD, the shift due to temperature was minimized. This information was used to select a density range for experiments in which temperature was controlled dynamically (see FIGS. 7A-7C). As cells may shift in size in response to heating, we also quantified temperature-induced offset in optical density readings using evaporated milk.

### Example 14. Interchangeable Fluidic Module for Liquid Handling

In this section, we describe the implementation of an additional board, separate from the main Motherboard, that we developed for fluidic control of each culture vessel. This design enables individual control over the liquid handling, just as Smart Sleeves enable individual control over other culture parameters. However, consolidating these components into a dedicated board facilitates changing between different modes of liquid handling.

Automated cell culture relies on programmable input/output of culture media. Fast and accurate, peristaltic pumps are typically used for this application (Toprak E., et al., Nat. Protoc. 8, 555-567 (2013)). For a single media input, a culture vessel requires two peristaltic pumps, one for influx and one for efflux. The influx line routes the media from the source into the culture, and the efflux line takes out waste media to maintain a fixed volume. Timing and coordination of these pumps is important for any automated cell culture application. A single input/single output system is the most basic type of fluidic control, and yet applying this scheme to a large number of independently-controlled culture vessels can prove challenging. To address this, and in anticipation of wanting to access even more complex fluidic functions, we developed a dedicated Auxiliary Board, separate from the Motherboard. The auxiliary board can simultaneously and independently control up to 48 fluidic control elements and supports much-needed abstraction of fluidic routines. The auxiliary board facilitates simple input/output functions at scale and accommodates more sophisticated fluidic solutions (see Example 15).

The auxiliary board is designed to receive serial inputs from the Raspberry Pi, translate abstract commands into simple sequential tasks, and simultaneously control up to 48 fluidic elements (FIGS. 17A-17B). The board contains many of the same components from the Motherboard (e.g. RS485, Arduinos, PWM) and serially communicates in the same manner. A typical 16-vial single media experiment, requiring the use of 32 control elements, consisting of two pumps per vial: one for influx and one for efflux.

In this study, we applied this common hardware architecture (FIG. 17A) to enable two modes of fluidic control in eVOLVER: (1) a “basic fluidic scheme”, wherein pairs of peristaltic pumps control the influx and efflux of media in each vial (FIG. 17A); (2) a “complex fluidic scheme”, wherein customizable integrated millifluidic devices with pneumatic valves are used to route fluid in a programmable manner to execute complex fluidic tasks (FIG. 17C, see Example 15).

In the basic eVOLVER setup featured in FIGS. 6A-6C and FIGS. 7A-7D, we constructed arrays of 12V peristaltic dosing pumps (Adafruit, Product ID: 1150), which are easily implemented and are a good compromise between speed, accuracy, and cost. With peristaltic pumps and the eVOLVER hardware framework, fluid flow rates can be controlled in two ways. First, controlling the duration of pump events permits metering out defined volumes according to calibration curves (FIGS. 18A-18B). Second, flow rate can be controlled using the PWM board to apply different current profiles in order to run the peristaltic pumps at different power levels. This is particularly useful when seeking lower flow rates, as minimum pump duration (˜0.5 seconds) is constrained by the rate of communication between boards, so reducing power level can increase precision at small dose sizes.

A small degree of scalability is possible with the basic fluidic scheme of using the auxiliary board to control individual peristaltic pumps for each fluidic line. For example, running 16 vials in a typical two-input experiment (such as morbidostat-like experiments (Toprak E., et al., Nat. Protoc. 8, 555-567 (2013): Toprak E., et al., Nat. Genet. 44, 101-105 (2011)) would utilize all 48 channels of the auxiliary board, with three pumps per vial: two for influx and one for efflux. To permit further scaling of fluidic tasks without significantly increasing the number of pumps necessary, we developed a different paradigm of millifluidic handling for automated cell culture (complex fluidic scheme), which utilizes fabricated pneumatically-valved integrated devices (see Example 15).

### Example 15. Integrated Millifluidic Devices for Complex Fluidic Control

A key development in microfluidics was the design and fabrication of devices containing integrated (pneumatic) valves that could allow for complex fluidic manipulations with minimal number of control elements (Unger M. A., Science 288, 113-116 (2000); Thorsen T., et al., Science 298, 580-4 (2002); Melin J. and Quake S. R., Annu. Rev. Biophys. Biomol. Struct. 36, 213-231 (2007)). Here, we describe (1) why adapting this technology for the macro scale is valuable for automated cell culture, (2) challenges faced when scaling to larger flow rates, and (3) a new framework for fabrication and bonding of millifluidic devices featuring integrated pneumatic valves. These devices offer a scalable solution to challenges faced by traditional fluidics.

Importance of Complex Fluidic Tasks:

The ability to program complex fluidic tasks could enable entirely new manipulations in automated cell culture applications (see FIGS. 8A-8C, Example 6). For example, when growing undomesticated microbes, biofilm may form in the efflux fluidic lines and vials in as little as in 12 hours (e.g. see FIGS. 8A-8C). The ability to programmatically bypass the vial in order to clean the fluidic lines with a bleach solution, and passage the culture from one vial to the next as a preventative measure, would be critical for long-term continuous growth of these microbes. However, complex fluidic tasks like vial-to-vial transfer, cleaning protocols, and mixed media inputs are extremely difficult with traditional fluidic systems used by current devices.

In electronics, custom circuits can be readily created by breadboarding; however, this approach scales poorly to larger, more complex circuits because it relies on tedious manual assembly and leads to limited durability. Similarly, fluidic systems consisting of flexible tubing connecting separate control elements, like pumps and valves, can solve simple fluidic tasks. However, the number of necessary fluidic connections scales with the complexity of the desired task. For example, even with an optimal valving scheme, the ability to perform automated large-volume transfers between any two culture vials in a 16-vial eVOLVER unit would require almost 300 fluidic connections and over 20 control elements. As in breadboarding, each connection would need to be routed individually by fluidic tubing and often by hand, a tedious task. Additionally, the tubing is usually fairly long, and each connection introduces dead volume, making the system less robust and impractical. Instead, by creating integrated (pneumatically-valved) schematics, we sought to make a millifluidic equivalent of a printed circuit board; the complex fluidic connections are now integrated in a small device that is computer designed, manufacturable, and much easier to reproduce. With the flexibility of CAD, one would be able to customize a fluidic device to fit their particular experimental needs.

Fluidic Scaling Problem:

The cost of control elements and assembly time of bioreactor units can prove to be a significant burden as one scales fluidic inputs and outputs for high-throughput operations. As previously described, most designs rely on a pinch valve or a peristaltic pump to separately control each of the media sources and another to control waste. For example, a single vial turbidostat unit with 4 different media inputs would require 5 pumps. A hypothetical 48-vial unit with the same capabilities would therefore require 240 pumps, at a cost of $7,000 to $10,000. The key problem is that the number of control elements increases linearly with the number of vials. Our pneumatically-valved devices can leverage concepts developed in microfluidics in order to scale throughput by multiplexing and demultiplexing inputs and outputs (Thorsen T., et al., Science 298, 580-4 (2002): Grover W. H., et al., Sensors Actuators, B Chem. 89, 315-323 (2003)). In this scheme, the number of controllable vials scales exponentially to the number of control elements, needing only 30 elements to route up to 16 different fluidic inputs to 48 vials. We project the costs of this new hypothetical 48 vial fluidic schematic to be $1,000 to $2,000, roughly a 90% decrease in cost in comparison to current systems.

Desired Characteristics and Properties of Integrated Millifluidic Devices:

Though inspired by microfluidic technologies, integrated millifluidic devices for continuous culture have drastically different design requirements. The following is a list of critical requirements for this technology in the context of continuous culture:
Devices need to be on the decimeter scale. Indicated in the nomenclature, microfluidic devices operate on the nano to micro liter per second flow rate. In contrast, continuous culture in eVOLVER requires flow rates of roughly ˜1 mL per second, a 1000-fold increase. As such, in order to increase flow rate with an appropriate safety factor, the flow channels and device needs to be at least 10-fold larger than typical microfluidic devices.
New prototyping framework is necessary for larger devices. Traditional microfluidic prototyping techniques rely on standards from the microelectronics industry, namely patterning photoresist on silicon wafers (Unger M. A., Science 288, 113-116 (2000); Duffy D. C., et al., Anal. Chem. 70, 4974-4984 (1998)). Typically, 100 mm (4 inch) circular wafers are the largest size the machinery can pattern designs on, which is still too small for complex millifluidic devices. New fabrication framework is necessary to prototype devices for continuous culture.
Design cycle must be fast and repeatable. The success of microfluidics in the laboratory is, in large part, attributed to rapid and reliable design cycles enabling iteration and testing of prototypes. New design frameworks for millifluidic devices must be equally rapid and reliable.
Device must be transparent. Fluidic systems designed for long-term continuous culture of microbes will be prone to biofilm formation. The ability to monitor flow through the device is critical for debugging experimental issues.
Device needs to interface with pumps, filters, and tubing. Fluidics for laboratory continuous culture typically interface with syringe pumps, pressurized fluids, sterile filters and peristaltic pumps. A robust way to interface dozens of connections between the device and other fluidic elements (e.g. vials, filters) is critical and nontrivial.
Device must be resistant to 10% bleach and 70% ethanol. Sterilization of the device is necessary prior to any experimentation. Fluidic materials and fabrication must be resilient to these chemicals for weeks of continuous usage.

Fabricating Integrated Millifluidic Devices for Automated Cell Culture:

Fabrication techniques used for microfluidics (e.g. photolithography, surface treatments) do not simply translate to larger dimensions. To scale devices to the millifluidic scale, an entirely new fabrication method is required. First, reagents for photolithography are optimized for channel heights of 1 to 300 microns. To reach the desired channel height of ˜1 mm would involve tediously stacking photoresist layers together, which requires precise alignment of photomasks. Second, the chemical glues, like silanes, that are typically used to functionalize plastic and silicone rubber sheets for bonding are difficult to apply uniformly across a large area (e.g. 10 cm×20 cm). Any small pocket where bonding was incomplete compromises the integrity of the entire device. Finally, the ability to prevent bonding in specific areas of the device (i.e. the pneumatic valves) is also critical, yet difficult with current techniques. Since there can be hundreds of integrated valves that must be protected from bonding, the ability to denote where the bonding occurs via a CAD drawing, instead of by hand, is critical to robust fabrication of the device.

To fulfill the design criteria previously listed, we developed a simple, robust prototyping method for fabrication and selective bonding of devices for fluidic control on the eVOLVER platform (FIGS. 19A-19C). We used a 40 W CO₂ laser cutter (Epilog Mini 24) to pattern clear PETG material. Laser cutters are easy to use, readily available at most universities, and can easily raster a pattern from a CAD drawing. PETG is commonly found in plastic water bottles and is chemically resistant to ethanol and bleach. The device is divided into two layers, the control layer (¼″ PETG) and the flow layer (⅛″ PETG), which sandwich a silicone membrane (0.01″, Rogers Corporation, BISCO HT-6240) between them when assembled.

For proper pneumatic valving in the devices, an airtight seal must be formed between all layers. To bond the layers together, an optically clear laminating adhesive sheet was used (3M, 8146-3). The adhesive comes as a sheet sandwiched between polyester backings to maintain integrity of the adhesive. First, the PETG layers are plasma treated for 1 min with atmospheric gasses at MAX setting (Harrick Plasma, 30 W Expanded Plasma Cleaner) to promote adhesion between the adhesive and plastic. Adhesive (with one side of the backing removed) is quickly placed onto the activated surface and any bubbles are quickly rolled out. The PETG sheets with adhesive are then patterned with a laser cutter. To get a deeper cut without melting the plastic, the same design was cut three times (20% Speed, 100% Power). For selective bonding of the device, low laser power is used to raster off the adhesive but not cut into the plastic (70% Speed, 50% Power). Bonding of the PETG layers to the silicone rubber is accomplished by plasma treating the silicone rubber sheet and subsequently applying the sheet onto the adhesive. Clamping the two layers between two ½″ metal plates immediately after plasma treatment helps in bonding the two surfaces.

To interface with the device, barbed-to-thread polypropylene connectors (Value Plastics, X220-6005) were fastened into 10-32 threaded holes on the thicker control layer. 3 mm vias were punched into the silicone membrane to connect the flow layer to the barbed connectors on the control layer. The entire fabrication process, from a CAD drawing to a completed device, can be done in 3 hours.

Characteristics of Pneumatic Valves for Millifluidic Devices:

Our pneumatically-valved millifluidic devices enable customizable, programmable routing of liquid at volumetric flow rates of ˜1 mL/sec. The valves pinch off fluid flow on the flow layer when 10 psi is applied to the control layer and enable flow when vacuum is applied (FIGS. 19A-19C). Improvement of device bonding will enable application of pressures above 10 psi, necessary with higher flow rates.

Coordinating New Fluidic Experimental Parameters:

Fluidic tasks in eVOLVER are enabled by the sequential actuation of valves in a specific fluidic network encoded in the integrated millifluidic device. We demonstrate these fluidic manipulations in a series of experiments (see FIGS. 8A-8C, Example 6). Each experiment utilizes different devices as required to meet the experimental needs. The architectures for most functions are modular (e.g. multiplexer, vial-to-vial router) and can be combined in order to achieve more complex functionalities (FIGS. 19A-19C). For example, simple single media input turbidostat function utilizes multiplexer and demultiplexer modules. The demultiplexer routes the media source to the correct vial and the multiplexer routes the efflux from vial to waste. The same multiplexer and demultiplexer modules are reused in all FIGS. 8A-8C applications, but different multiplexed media selectors and vial-to-vial routers are included as needed in different experiments.

Software routines to control the control elements (valves and pumps) are also divided into commonly repeated functions, usually in a similar manner to how fluidic modules were divided. The code for each fluidic function is preloaded into the Arduino to coordinate tasks between each fluidic module. For example, a simple dilution event would first actuate valves in the media multiplexer to select media, then actuate a syringe pump for metering the desired volume, followed by valves in the vial demultiplexer and multiplexer to route media into the vial and remove efflux. By loading the routine for abstract functions (e.g. dilute, clean, vial-to-vial transfer) into the Arduino, robust communication can be ensured, with rapid transition between sub-tasks and no skipped steps (which could cause incorrect media routing, mis-priming of the syringe pump, or leaks and other device failures).

### Example 16. Reconfiguration of eVOLVER for Common Culture Experiments

Rather than propose a single continuous culture device designed to a specific purpose, our goal with eVOLVER was to demonstrate a design framework that gives the user the freedom to imagine and carry out virtually any type of experiment that uses automated cell growth functionality to study cellular fitness. Here, we comment on reconfiguring eVOLVER for several experiments of significant interest in the community. For further examples, see

TABLE 1
Reconfiguring eVOLVER for common continuous growth and evolution applications.

| Reconfiguring eVOLVER | | | | | | |
|---|---|---|---|---|---|---|
| for Recent Continuous | | | | | | |
| Growth and Evolution | | | Description of | | | Estimated |
| Applications | | Description | eVOLVER | Hardware | Software | Time/Cost |
| Study | Experiment | of Goals | Setup | Modifieations | Changes | to Modify |
| Toprak et | Morbidostat | Dynamic | Standard sleeve | None, achievable | Minimal, | Works as is |
| al. 2011 | | feedback | configuration, | using components | easily | (code |
| | | control over | but use additional | reported in paper | programmed | available) |
| | | two fluidic | input pump array | | in Python | |
| | | inputs based | or millifluidic | | | |
| | | on optical | devices for two | | | |
| | | density | inputs | | | |
| | | measurements | | | | |
| Takahashi | Real-time | On-line | Use open slot for | New SA boards, | Minimal, | <1 month, |
| et al. 2015 | Fluorescence | fluorescence | microcontroller | additional LED/ | based on | <$2000 |
| | measurements | detector for | and new | sensor pair | optical density | |
| | | measuring | PWM/ADC | | code reported | |
| | | fluorescence | boards adapted | | in paper | |
| | | directly in | from OD control | | | |
| | | culture | boards to control | | | |
| | | chamber | new LED/sensor | | | |
| | | | pair integrated | | | |
| | | | into smart sleeve | | | |
| Milias- | Optogenetic | LED of | Use open slot for | Additional | Minimal, | <1 month, |
| Argeitis | Control | particular | microcontroller | microcontroller | based on | <$50 |
| et al. 2016 | | wavelength | and PWM board | and PWM board, | optical density | (Prototype |
| | | used to induce | to control new | new LED | code reported | implemented) |
| | | changes in | LED integrated | integrated in | in paper | |
| | | light-sensitive | into smart sleeve | smart sleeve | | |
| | | proteins within | | | | |
| | | culture vessel | | | | |
| Hope et | HT ministat | 96 parallel | Same as | None | Minimal, | Works as is |
| al. 2017 | array | chemostats | described in | | set | |
| | | | paper, using six | | repetitive | |
| | | | eVOLVER 16- | | dilutions | |
| | | | vial units | | using | |
| | | | | | Python for | |
| | | | | | coarse- | |
| | | | | | grained | |
| | | | | | control or | |
| | | | | | Arduino for | |
| | | | | | fine-grain tied | |
| | | | | | control | |
| Esvelt et | PACE: | Provide | Several | Modify | Fair, | 3 months, |
| al. 2011 | Accelerated | higher input | eVOLVER | peristaltic | program | <$500 |
| | Evolution | control and | sleeves in | pumps to | appropriate | |
| | | throughput | chemostat mode | achieve | flow rates | |
| | | for rounds of | house the | desired flow | and new | |
| | | phage- | bacterial | rates in and | abstractions | |
| | | assisted | substrate for the | out of | for | |
| | | continuous | phage to infect. | chemostats | millifluidic | |
| | | | Peristaltic pumps | and lagoons. | devices. | |
| | | | will meter in a | Multiplexed | | |
| | | | constant amount | millifluidic | | |
| | | | of the substrate | devices will | | |
| | | | into different | contain | | |
| | | | phage-containing | valving | | |
| | | | lagoons, housed | necessary to | | |
| | | | in the remaining | tune inducer | | |
| | | | eVOLVER | flow rate and | | |
| | | | sleeves, | concentration | | |
| | | | Millifluidic | over time. | | |
| | | | devices will | | | |
| | | | control precise | | | |
| | | | amounts of | | | |
| | | | inducer entering | | | |
| | | | the culture, to | | | |
| | | | tune activity of | | | |
| | | | lagoon. | | | |

Chemostat:

As one of the simplest forms of continuous culture, small-volume (˜mL) chemostat arrays have been popular in directed and experimental evolution (Hope E. A., et al., Genetics 206, 1153-1167 (2017); Esvelt K. M., Nature 472, 499-503 (2011)). To run eVOLVER as a chemostat, one would use the peristaltic pump array with dilution events triggered by a programmed timer, rather than by optical density (as in turbidostat mode). This alteration can simply be made, without hardware changes, on the Python code (see FIGS. 45A-45B). Each vial in eVOLVER can be programmed with a different dilution rate. Alternatively, more rapid communication can be achieved by modifying the code on the auxiliary board Arduino that controls the fluidic channels. In more detail, the pumps described in the manuscript have a fixed flow rate of (˜1 mis); however, by varying frequency and duration of turning the pump ON, one could achieve a lower average target flow rate. For example, by turning on the pump for 1 second every 10 seconds, a flow rate of 100 uL/s can be reached with a continuous approximation. The input pump can then be flexibly and dynamically programmed to achieve different rates. The pump can robustly fire for as short as 0.5 seconds (for a bolus of ˜0.5 mL). If the application requires slower or more continuous flow, pumps can also be switched out for slower motors or tubing diameters. Any peristaltic pumps pulling less than 0.5 amp/channel could be controlled by the Auxiliary board.

Morbidostat:

Morbidostat algorithms have been developed that gradually increase the selection pressure of an evolving culture, typically based on measured growth rate (Toprak E., et al., Nat. Genet. 44, 101-105 (2011)). Previously, this algorithm has been implemented with two media inputs (+ and − drug), requiring three peristaltic pumps per culture (w/efflux pump). In a 16-vial eVOLVER unit, this setup can easily be implemented by (1) controlling 48 pumps with the auxiliary board or (2) using multiplexed fluidics with the millifluidic devices. The prior being simpler to implement for 2 media inputs and the latter letting one scale to >2 inputs. As currently designed, the auxiliary board can control up to 48 fluidic elements (pumps/solenoids). To run morbidostat mode, one would need to modify the Python code to the desired growth algorithm (e.g. control rate of drug increase, growth rate threshold to trigger the drug input).

Optogenetic Control During Continuous Culture:

Light inducible protein domains have been used to dynamically control and rapidly prototype genetic networks (Milias-Argeitis A., Nat. Commun. 7, 12546 (2016): Olson E. J., et al., Nat. Methods 11, 449-455 (2014)). Hardware for light inducible systems typically rely on batch culture, limiting experiments to a narrow time window in which all cells across an experiment are in exponential phase. Attempts at coupling continuous culture to light induction have been limited by throughput (1-2 cultures) and reconfigurability. Equipped with components for light induction, eVOLVER would uniquely enable long-term optogenetic perturbations in finely controlled growth phases across a large number of culture vessels. Due to the modularity of eVOLVER hardware components, integrating optogenetic control is straight forward and requires minor modifications to the system. Details on integrating LEDs into eVOLVER is described in more detail as the example modification in Example 13.

Fluorescence Measurements:

Bulk fluorescence measurements have previously been demonstrated by Takahashi et al. during continuous culture, without the use of a photomultiplier tube (PMT) (Takahashi C. N., ACS Synth. Biol. 4, 32-38 (2015)). To recapitulate this setup in eVOLVER, an extra LED-diode pair would be added to the 6th and 7th S/A Slots, similar to adding LEDs for light induction. Additionally, the 3D printed part would be modified to house optical filters for better detection of any fluorescence signal. Potential setbacks in this setup (without a PMT) include potentially a low signal to noise ratio. This can be solved by multiplexing signal from all cultures into a single PMT via fiber optics. The electronics controlling the PMT would communicate back to the same RS485 line to be controlled by the same Raspberry Pi, similar to the auxiliary board. Alternatively, single cell fluorescence measurements would also be made possible by interfacing eVOLVER with a pipetting robot, droplet microfluidics, or using the native pump from the flow cytometer sample directly from the cultures. These systems could interface serially with the Raspberry Pi via RS485/USB or the lab computer via USB.

Different Culture Volumes:

As described in this manuscript. 40 mL culture vessels were chosen for a sufficiently large population size for full coverage of the genome during evolution. Other applications like bioproduction, larger library screens, or applications with expensive culture medium might be better suited with alternative volumes. The modularity of the eVOLVER framework enables redesign of the Smart Sleeve with limited changes to the rest of the hardware. For example, to design a sleeve for larger volumes, one would (1) machine a new aluminum casing, (2) obtain a fan/motor capable of stirring a larger/smaller volume, and (3) redesign a PCB/3D printed piece to optimize position of components (orientation of diode/LED for O.D. measurements through larger volume). The rest of the hardware downstream of the Smart Sleeve could potentially remain the same or have only slight software modifications, depending on size of culture vessel (e.g. tune PID controller for larger thermal mass).

### Example 17. Common Considerations when Running eVOLVER

With the appropriate parts (e.g. boards, electronic components etc.) and appropriate DIY manufacturing equipment (e.g. 3D printer, laser cutter), a 16-vial platform could be built in a week for $5,000. However, beyond assembly of an eVOLVER device itself, there are several other challenges to be considered prior to routine use of eVOLVER.

Media Requirements:

A primary consideration when running eVOLVER experiments is predicting the media consumption rate, which is typically orders of magnitude higher than in typical batch experiments. The required media for continuous culture can be estimated using the following equation:

$$Media Required [ mL ] = \frac{Culture Volume [ mL ]}{Avg . Doubling Time [ h ]} * Number of Cultures * Duration [ h ] \qquad ( 1 )$$

It should be noted that additional media (˜20 mL) is needed during device setup to flush media input lines.

Network Connectivity:

The simplest eVOLVER setup communicates to the lab computer (running Python) within a local network via an Ethernet connection. Examples of the local network are a user's personal router or the building's router at the institution. It is recommended to contact local IT services for site-specific configurations. This setup does not require connectivity to the internet but is convenient to have. Empirically, we have observed disruption in communication with eVOLVER during and immediately after use of remote desktop software to access the lab computer, use of this software is not recommended with eVOLVER. A more scalable, reliable solution (running ˜100s of vials) would be to use a dedicated server to host the Python scripts and visualizations software (instead of a lab computer). These cloud services can be hosted by the university or externally via commercial providers (e.g. Amazon Web Services) and accessed on a browser on any computer. Again, we recommend contacting IT specialists for secure methods of communication between eVOLVER and the server at your institution. An Ethernet connection (instead of Wi-Fi) is recommended for all configurations.

Maintenance:

We have monitored the lifetime of eVOLVER components since the invention of the device (˜3 years). As in most systems, mechanical parts have the highest possibility of wear and thus need replacement most frequently. Likewise, in eVOLVER, the peristaltic pumps used have a lifetime of ˜6 months of typical use during continuous culture. Specifically, the silicone tubing within the head of the pump is frequently compressed when actuating peristaltic pumps and will tear over time. The head can easily and inexpensively be replaced (˜$4/pump). The computer fan used for stirring has robustly operated continuously for >3 years, whereas the magnetic stir bars are rated for a limited number of autoclave cycles and need eventual replacement. All other components (e.g. heaters, thermistors. LEDs, diodes, PCB, power sources) have been stably operating for >3 years.

Contamination Prevention:

While batch culture techniques often utilize biosafety hoods or flame convection currents to keep workspaces sterile, these are rarely amenable for automated cell culture devices. Prevention of contamination in eVOLVER is achieved at three levels: 1) sterilization of media, culture vessels, and fluidic lines, 2) attention to sterile technique, and 3) physical and chemical barriers to contaminants. First, all media bottles and their adapters, and all components of the culture vessel (e.g. borosilicate glass vial, magnetic stir bar, cap with fluidic adapters) are designed to be autoclaved before each use. Fluidic lines on the device are sterilized before and after each experiment using bleach and ethanol (see Example 6). Second, following sterilization, sterile technique should be practiced when attaching media lines to culture vials by working quickly, avoiding physical contact with the ends of fluidics lines, and taking care to spray gloves with ethanol. Finally, additional physical and chemical measures may be taken depending on the organism and experiment. For example, the sampling port may be covered by a sterile membrane for long-term culture. For slow-growing eukaryotic cultures, antibiotics can be added to the media to exclude bacterial contaminants. UV sterilization of components or surfaces is another preventative measure to consider.

Safety while Operating eVOLVER:

eVOLVER electronics modules are connected to high amperage power supplies. Electronic components may be exposed if improperly constructed and extreme care should be taken to protect against shock.

### Example 18. Open-Source Frameworks for Biology

Many biological experiments require varying amounts of customization to fit the experimental needs of the researcher. However, currently, most laboratory equipment is single-use, proprietary and expensive, making customization difficult. Consequently, even simple experiments are usually designed around the capabilities of the equipment, at times severely limiting how experiments are run. In the last decade, do-it-yourself (DIY), open-source electronics have risen significantly in popularity due to falling costs of printed circuit board (PCB) manufacturing and the maturing open-source community. This cultural shift has resulted in creative, useful, and customizable tools such as 3D printers and small Linux computers (Raspberry Pi). These open-sourced technologies have changed how manufacturing, networking, and prototyping are approached, making it easier to build tools to exactly fit one's needs.

Similarly, the DIY culture has the potential to significantly impact biological experimentation. In fact, university labs are well suited to driving innovation with open sourced lab tools due to: (1) local technical competence, (2) resources (e.g. infrastructure, financial), (3) highly specific/diverse experimental needs, and (4) desire to share and replicate one's work. Despite these apparent synergies, DIY tools have not been widely used in biological laboratories, and ad hoc tools built in academic labs are often not robust enough for widespread adoption; often this decision comes down to valuing robustness over customizability. In order for DIY tools to be reliable, there must be a framework or standard that is robust, yet still allows flexibility and creativity for the user. For example, Arduino boards are a hardware framework/footprint to house a common micro-controller (ATmega328P). This framework became a critical standard for the community and allows hobbyists to easily program and interface with the micro-controller for their own application (e.g. drones, 3D printers). With this and other DIY successes in mind, we sought to develop eVOLVER as a modular, open-sourced hardware framework that is robust enough for widespread adoption, while still customizable for particular applications.

### Example 18. Discussion

eVOLVER achieves the goal of creating a standardizing framework for automated cell growth experiments. The system is designed from the bottom-up to be customizable and expandable; its DIY infrastructure provides researchers with the ability to design, easily build out, and share new experimental configurations and data. eVOLVER's design utility is manifested at the component level: in Smart Sleeve design configurability, in the ability to specify custom liquid manipulation routines using the fluidic system, and in the modularity and composability of the hardware and software systems (FIGS. 4A-4D and FIGS. 5A-5C). As a consequence, with straightforward modification, eVOLVER can be reconfigured to conduct any of the recently reported continuous growth studies (TABLE 1), or could replace tedious batch culture techniques used in a number of recent experimental evolution studies (Mitchell A., et al., Nature 460, 220-224 (2009); Lang G. I., et al., Nature 500, 571-574 (2013); Yona A. H., et al., Proc. Natl. Acad. Sci. U.S.A 109, 21010-5 (2012); Gonzalez C., et al., Mol. Syst. Biol. 11, 827 (2015)). Additionally, new hardware components can easily be incorporated into the platform. For example, integration with emerging open-source pipetting robots would automate culture sampling, unlocking downstream fluorescence-activated cell sorting (FACS) for assaying gene expression, or droplet microfluidics for single-cell studies. The present configuration is designed for well-mixed liquid cultures, but the eVOLVER control framework could be adopted for the coordination of multiple arrayed sensors to capture spatial distributions in static liquid cultures or phototrophic cultures. Finally, while eVOLVER is well suited for hardy, fast-growing suspension cultures of microbes, additional attention to sterility and removal of residual cleaning agents would be needed for sensitive mammalian lines, and bead/matrix systems may be needed for adherent cells.

The results reported in this study establish eVOLVER as a scalable framework for realizing large-scale, multidimensional selection experiments to study, characterize, and evolve biological systems. The system's configurability enables precise specification of culture environment on an individual culture basis. By systematically co-varying parameters, eVOLVER can be used to investigate cellular fitness along multidimensional environmental gradients, potentially allowing for experimental decoupling of overlapping selection pressures. The ability to arbitrarily program feedback control between culture conditions and fluidic functions allows the user to algorithmically define highly specialized environmental niches.

We demonstrated the breadth of eVOLVER's experimental versatility in a series of showcase experiments. First, we used eVOLVER to conduct an experimental evolution study (FIGS. 6A-6C). There has been longstanding interest in the interplay between environmental carrying capacity, growth rate, and population size (Luckinbill L. S. R., et al., Science 202, 1201-3 (1978); Wahl LM., et a., Genetics 162, 961-971 (2002); Lande R., et al., Philos. Trans. R. Soc. B Biol. Sci. 364, 1511-1518 (2009)). In our study, we evolved yeast across 78 different culture density windows. We then generated fitness distributions by testing fitness of evolved clones in low- and high-density niches, identifying low- and high-density specialists. Interestingly, high-density specialists were most often derived from evolution in narrow OD windows. Since the prescribed culture density windows are related to the frequency of diauxic shift at limiting glucose concentrations (Brauer M. J., et al., Mol. Biol. Cell 16, 2503-17 (2005)), it is interesting to speculate that these strains selected for metabolic programs that facilitate rapid metabolic shifts. Critically, further work is needed to confirm that the differences observed in the fitness distributions are indeed significant. First, a baseline fitness distribution generated from a large number of replicate evolutions could be used to rule out the possibility that stochastic events dominate the observed fitness differences. Additionally, comparing the fitness of whole evolved populations in each condition could help isolate true adaptation from variation observed due to clonal differences. Finally, assaying fitness in additional niches is required to determine how well fitness distributions correlate with the assayed niche, as well as to ascertain the existence of generalists. Nevertheless, this experiment demonstrates how eVOLVER's high-throughput capabilities can be used to uncover subtly varied adaptations that likely would have been obscured by either a batch culture or a lower-resolution automated approach. Furthermore, eVOLVER enables rich phenotypic profiles to be constructed from individual culture histories using data collected during long term experiments (e.g. growth rate, genome replications).

In a second experiment, we performed growth fitness experiments on a YKO library under systematically varied temperature fluctuations, demonstrating that eVOLVER could be used to extract unique fitness information across a temporally varied selection surface (FIGS. 7A-7D). In a similar approach-using sub-lethal stress administered under temporally diverse selection conditions-selection experiments could be conducted for other types of libraries at a resolution beyond what is available for standard growth selection schemes (Giaever G. et al., Nature 418, 387-391 (2002); Smith A. M. et al., Genome Res. 19, 1836-1842 (2009); Gibney P. A., et al., Proc. Natl. Acad. Sci. 110, E4393-E4402 (2013)). Using this approach, new algorithms with additional parameters can be used to perform fitness and selection experiments investigating biological phenomenon associated with temporal adaptation, like bet-hedging, noise, and transcriptional feedback.

Accurate fluidic manipulation is a critical feature of continuous culture automation, but past approaches to fluidic routing have imposed experimental limitations. Inspired by electronic and microfluidic technologies (Thorsen T., et al., Science 298, 580-4 (2002)), we addressed this issue by developing a novel millifluidic control paradigm for programmatic routing of fluids during continuous culture. Our devices expand the repertoire of cellular manipulations available to eVOLVER while packaging functionality into a compact physical footprint. To highlight the utility and robustness of the devices, we performed three experimental demonstrations: sophisticated fluidic mixing and dispensation, vial-to-vial transfers, and integration of multiple devices for more complex culture routines (FIGS. 8A-8C). These experiments illustrate the potential of custom millifluidics to create sophisticated algorithmic selection routines. As we demonstrate, eVOLVER can be configured to culture separate strains, simultaneously intermixing them to study community interactions. For microbial populations capable of exchanging genetic information, such an approach could be used to explore different paths by which resistance can spread, with implications for infectious disease treatment.

We foresee the eVOLVER platform as an enabling tool for several emerging fields of research. eVOLVER could play an important role in investigating the adaptive basis of social behavior in microbial consortia. For example, the system's throughput, control, and fluidic capabilities could be leveraged to systematically test contributions of individual species to community fitness, potentially offering insight into how to construct ecologically stable communities from the bottom up (Friedman J., et al., Nat. Ecol. Evol. 1, 109 (2017)). In synthetic biology, designing regulatory circuits that minimize fitness cost to the host cell remains a major challenge³. Leveraging its ability to carefully monitor population fitness in high throughput, eVOLVER could be used to identify circuit design features that maximize evolutionary stability. This would be useful for applications requiring engineered cells to retain circuit function over many generations, and would be especially valuable for high-throughput testing of bioproduction strains prior to scaling-up to industrial bioreactors (Alper H., et al., Proc. Natl. Acad. Sci. U.S.A 102, 12678-83 (2005)). Relatedly, eVOLVER could be used to aid in the design, testing and optimization of synthetic microbial genomes (Hutchison C. A., et al., Science 351, aad6253-aad6253 (2016); Richardson S. M., et al., Science 355, 1040-1044 (2017)). Finally, we envision eVOLVER as enabling platform for building a community of users that can freely conceive, build, execute, and share experiments.

### REFERENCES

1. Toprak E., Veres A., Michel J. B., Chait R., Hartl D. L. Kishony R., Evolutionary paths to antibiotic resistance under dynamically sustained drug selection, Nat. Genet., 2011, 44(1): p. 101-5.
2. Acar M., Mettetal J. T., van Oudenaarden A., Stochastic switching as a survival strategy in fluctuating environments, Nat. Genet., 2008, 40(4): p471-75.
3. Klavins Lab Trubidostat Wiki, https://depts.washington.edu/soslab/turbidostat/pmwiki/(last visited Nov. 23, 2016).
4. Evolvinator-OpenWetWare, http://openwetware.org/wiki/Turbidostat (last visited Nov. 23, 2016).
5. Elena, S. F. & Lenski, R. E. Microbial genetics: Evolution experiments with microorganisms: the dynamics and genetic bases of adaptation. Nat. Rev. Genet. 4, 457-469 (2003).
6. Nichols, R. J. et al. Phenotypic Landscape of a Bacterial Cell. Cell 144, 143-156 (2011).
7. Nevozhay, D., Adams, R. M., Van Itallie, E., Bennett, M. R. & BalAzsi, G. Mapping the Environmental Fitness Landscape of a Synthetic Gene Circuit. PLoS Comput. Biol. 8, e1002480 (2012).
8. Giaever, G. et al. Functional profiling of the Saccharomyces cerevisiae genome. Nature 418, 387-391 (2002).
9. Li, Z. et al. Systematic exploration of essential yeast gene function with temperature-sensitive mutants. Nat. Biotechnol. 29, 361-7 (2011).
10. Zuleta, I. A., Aranda-Diaz, A., Li, H. & El-Samad, H. Dynamic characterization of growth and gene expression using high-throughput automated flow cytometry. Nat. Methods 11, 443-8 (2014).
11. Takahashi, C. N., Miller, A. W., Ekness, F., Dunham, M. J. & Klavins, E. A low cost, customizable turbidostat for use in synthetic circuit characterization. ACS Synth. Biol. 4, 32-38 (2015).
12. Feist, A. M., Herrgird, M. J., Thiele, I., Reed, J. L. & Palsson, B. Ø. Reconstruction of biochemical networks in microorganisms. Nat. Rev. Microbiol. 7, 129-143 (2008).
13. Lang, G. I. et al. Pervasive genetic hitchhiking and clonal interference in forty evolving yeast populations. Nature 500, 571-574 (2013).
14. Maddamsetti, R. et al. Adaptation, Clonal Interference, and Frequency-Dependent Interactions in a Long-Term Evolution Experiment with Escherichia coli. Genetics 200, 619-31 (2015).
15. Yona, A. H. et al. Chromosomal duplication is a transient evolutionary solution to stress. Proc. Natl. Acad. Sci. U.S.A 109, 21010-5 (2012).
16. Wang, H. H. et al. Programming cells by multiplex genome engineering and accelerated evolution. Nature 460, 894-898 (2009).
17. Esvelt, K. M., Carlson, J. C. & Liu, D. R. A system for the continuous directed evolution of biomolecules. Nature 472, 499-503 (2011).
18. Ravikumar, A., Arrieta, A. & Liu, C. C. An orthogonal DNA replication system in yeast. Nat. Chem. Biol. 10, 175-177 (2014).
19. Crook, N. et al. In vivo continuous evolution of genes and pathways in yeast. Nat. Commun. 7, 13051 (2016).
20. Bull, A. T. The renaissance of continuous culture in the post-genomics age. J. Ind. Microbiol. Biotechnol. 37, 993-1021 (2010).
21. Gresham, D. & Dunham, M. J. The enduring utility of continuous culturing in experimental evolution. Genomics 104, 399-405 (2014).
22. Piper, M. D. W. et al. Reproducibility of oligonucleotide microarray transcriptome analyses. An interlaboratory comparison using chemostat cultures of Saccharomyces cerevisiae. J. Biol. Chem. 277, 37001-37008 (2002).
23. Cressey, D. The DIY electronics transforming research. Nature 544, 125-126 (2017).
24. Kong, D. S. et al. Open-source, community-driven microfluidics with Metafluidics. Nat. Biotechnol. 35, 523-529 (2017).
25. Adamo, A. et al. On-demand continuous-flow production of pharmaceuticals in a compact, reconfigurable system. Science 352, 61-7 (2016).
26. Perkel, J. M. The Internet of Things comes to the lab. Nature 542, 125-126 (2017).
27. Milias-Argeitis, A., Rullan, M., Aoki, S. K., Buchmann, P. & Khammash, M. Automated optogenetic feedback control for precise and robust regulation of gene expression and cell growth. Nat. Commun. 7, 12546 (2016).
28. Hope, E. A. et al. Experimental Evolution Reveals Favored Adaptive Routes to Cell Aggregation in Yeast. Genetics 206, 1153-1167 (2017).
29. Toprak, E. et al. Building a morbidostat: an automated continuous-culture device for studying bacterial drug resistance under dynamically sustained drug inhibition. Nat. Protoc. 8, 555-567 (2013).
30. Thorsen. T., Maerkl, S. J. & Quake, S. R. Microfluidic large-scale integration. Science 298, 580-4 (2002).
31. Melin, J. & Quake, S. R. Microfluidic Large-Scale Integration: The Evolution of Design Rules for Biological Automation. Annu. Rev. Biophys. Biomol. Struct. 36, 213-231 (2007).
32. Unger, M. A. Monolithic Microfabricated Valves and Pumps by Multilayer Soft Lithography. Science 288, 113-116 (2000).
33. Grover, W. H., Skelley, A. M., Liu, C. N., Lagally, E. T. & Mathies, R. A. Monolithic membrane valves and diaphragm pumps for practical large-scale integration into glass microfluidic devices. Sensors Actuators, B Chem. 89, 315-323 (2003).
34. Kryazhimskiy. S., Rice, D. P., Jerison, E. R. & Desai, M. M. Global epistasis makes adaptation predictable despite sequence-level stochasticity. Science 344, 1519-1522 (2014).
35. Mitchell, A. et al. Adaptive prediction of environmental changes by microorganisms. Nature 460, 220-224 (2009).
36. Ketola, T. et al. Fluctuating Temperature Leads To Evolution Of Thermal Generalism And Preadaptation To Novel Environments. Evolution (N. Y). 67, 2936-2944 (2013).
37. Sether, B.-E. & Engen, S. The concept of fitness in fluctuating environments. Trends Ecol. Evol. 30, 273-281 (2015).
38. Breslow, D. K. et al. A comprehensive strategy enabling high-resolution functional analysis of the yeast genome. Nat. Methods 5, 711-718 (2008).
39. Giaever, G. & Nislow. C. The yeast deletion collection: A decade of functional genomics. Genetics 197, 451-465 (2014).
40. Smith, A. M. et al. Quantitative phenotyping via deep barcode sequencing. Genome Res. 19, 1836-1842 (2009).
41. Gibney. P. A., Lu, C., Caudy, A. A., Hess. D. C. & Botstein, D. Yeast metabolic and signaling genes are required for heat-shock survival and have little overlap with the heat-induced genes. Proc. Natl. Acad. Sci. 110, E4393-E4402 (2013).
42. Morano, K. A. et al. The response to heat shock and oxidative stress in Saccharomyces cerevisiae. Genetics 190, 1157-95 (2012).
43. Cherry, J. M. et al. Saccharomyces Genome Database: the genomics resource of budding yeast. Nucleic Acids Res. 40, D700-D705 (2012).
44. Escalante-Chong, R. et al. Galactose metabolic genes in yeast respond to a ratio of galactose and glucose. Proc. Natl. Acad. Sci. U.S.A 112, 1636-41 (2015).
45. Rice, S. a et al. Biofilm Formation and Sloughing in Serratia marcescens Are Controlled by Quorum Sensing and Nutrient Cues. J. Bacteriol. 187, 3477-3485 (2005).
46. Anderson, J. B., Sirjusingh, C. & Ricker, N. Haploidy, diploidy and evolution of antifungal drug resistance in Saccharomyces cerevisiae. Genetics 168, 1915-23 (2004).
47. Gonzalez, C. et al. Stress-response balance drives the evolution of a network module and its host genome. Mol. Syst. Biol. 11, 827 (2015).
48. Luckinbill, L. S. r and K Selection in Experimental Populations of Escherichia coli. Science 202, 1201-3 (1978).
49. Wahl, L. M., Gerrish, P. J. & Saika-Voivod, I. Evaluating the impact of population bottlenecks in experimental evolution. Genetics 162, 961-971 (2002).
50. Lande, R., Engen, S. & Saether, B.-E. An evolutionary maximum principle for density-dependent population dynamics in a fluctuating environment. Philos. Trans. R. Soc. B Biol. Sci. 364, 1511-1518 (2009).
51. Brauer, M. J., Saldanha, A. J., Dolinski, K. & Botstein, D. Homeostatic adjustment and metabolic remodeling in glucose-limited yeast cultures. Mol. Biol. Cell 16, 2503-17 (2005).
52. Friedman, J., Higgins, L. M. & Gore, J. Community structure follows simple assembly rules in microbial microcosms. Nat. Ecol. Evol. 1, 109 (2017).
53. Alper, H., Fischer, C., Nevoigt, E. & Stephanopoulos, G. Tuning genetic control through promoter engineering. Proc. Natl. Acad. Sci. U.S.A 102, 12678-83 (2005).
54. Hutchison, C. A. et al. Design and synthesis of a minimal bacterial genome. Science 351, aad6253-aad6253 (2016).
55. Richardson, S. M. et al. Design of a synthetic yeast genome. Science 355, 1040-1044 (2017).
56. Duffy, D. C., McDonald, J. C., Schueller, O. J. A. & Whitesides, G. M. Rapid Prototyping of Microfluidic Systems in Poly(dimethylsiloxane). Anal. Chem. 70, 4974-4984 (1998).
57. Olson, E. J., Hartsough, L. A., Landry. B. P., Shroff, R. & Tabor, J. J. Characterizing bacterial gene circuit dynamics with optically programmed gene expression signals. Nat. Methods 11, 449-455 (2014).
58. Berry, D. B. et al. Multiple Means to the Same End: The Genetic Basis of Acquired Stress Resistance in Yeast. PLoS Genet. 7, e1002353 (2011).
59. Winzeler, E. A. et al. Functional characterization of the S. cerevisiae genome by gene deletion and parallel analysis. Science 285, 901-906 (1999).
60. Nguyen-Huu, T. D. et al. Timing and Variability of Galactose Metabolic Gene Activation Depend on the Rate of Environmental Change. PLoS Comput. Biol. 11, e1004399 (2015).
61. de Crécy-Lagard, V. A., Bellalou, J., Mutzel, R. & MarliBre, P. Long term adaptation of a microbial population to a permanent metabolic constraint: overcoming thymineless death by experimental evolution of Escherichia coli. BMC Biotechnol. 1, 10 (2001).
62. de Crécy, E. et al. Development of a novel continuous culture device for experimental evolution of bacterial populations. Appl. Microbiol. Biotechnol. 77, 489-496 (2007).
63. McDonald, M. J., Rice, D. P. & Desai, M. M. Sex speeds adaptation by altering the dynamics of molecular evolution. Nature 531, 233-236 (2016).
64. Kanafani, Z. A. & Perfect, J. R. Resistance to Antifungal Agents: Mechanisms and Clinical Impact. Clin. Infect. Dis. 46, 120-128 (2008).
65. Brook, I. Inoculum effect. Rev. Infect. Dis. 11, 361-8
66. Cokol, M. et al. Systematic exploration of synergistic drug pairs. Mol. Syst. Biol. 7, 544 (2011).
67. Osherovich, L. Z. & Weissman, J. S. Multiple Gln/Asn-rich prion domains confer susceptibility to induction of the yeast [PSI(+)] prion. Cell 106, 183-94 (2001).

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or.” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example. “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B,” the disclosure also contemplates the alternative embodiments “a composition consisting of A and B” and “a composition consisting essentially of A and B.”

1. A method of performing experimental evolution on at least one fluidic microbial culture in a continuous culture system comprising:
subjecting the at least one microbial culture to a dynamic environment, wherein the at least one microbial culture is exposed to a stress ramp function which is overlaid on top of a culture fitness function; and
increasing the amount of stress applied to the at least one microbial culture in response to the increased fitness of the at least one microbial culture, wherein fitness is calculated in real-time.

2. The method of claim 1, wherein the culture fitness function comprises more than one fitness measurement.

3. The method of claim 1, wherein the culture fitness function comprises a turbidity and/or a fluorescence measurement.

4. The method claim 1, wherein the stress ramp function comprises more than one microbial stress.

5. The method of claim 1, wherein the stress ramp function comprises an antibiotic, an antiseptic, a temperature, an aerobic, an anaerobic, an infectious, a nutrient, an irradiative, a pH, a metabolic, and/or a mechanical stress.

6. The method of claim 1, wherein the stress ramp function comprises an increase or decrease in temperature.

7. The method of claim 1, wherein the at least one microbial culture evolves a novel functionality, optionally wherein the novel functionality is selected from the group consisting of stress tolerance, nutrient utilization, and metabolite production.

8. (canceled)

9. The method of claim 1, wherein one or more of the at least one fluidic microbial cultures comprises:
an archaea, a bacterium, a fungi, a protista, a microbial merger or symbiont, and/or a planarian;
a suspension of mammalian cells, plant cells, or insect cells: or
a combination thereof.

10. (canceled)

11. The method of claim 1, wherein the continuous culture system:
comprises integrated wetware, hardware, and software modules that can be readily interchanged and reconfigured;
comprises a turbidostat with fluorescence detection;
is configured to allow for vial-to-vial culture transfer;
is configured to allow for continuous mixing of the microbial suspensions; or
a combination thereof.

12.-14. (canceled)

15. A method of testing the mutational stability of an engineered circuit, said method comprising:
subjecting a microbial cell comprising at least one engineered circuit to a dynamic environment, wherein the microbial cell is exposed to stress ramp function which is overlaid on top of a culture fitness function;
increasing the amount of stress applied to the microbial cell in response to the increased fitness of the microbial cell, wherein fitness is calculated in real-time; and
determining the time required for the engineered circuit to inactivate.

16. The method of claim 15, wherein the engineered circuit comprises a fluorescent output, optionally wherein the fluorescent output is selected from the group consisting of TagBFP, mTagBFP2, Azurite, EBFP2, mKalama1, Sirius, Sapphire, T-Sapphire, ECFP, Cerulean, SCFP3A, mTurquoise, mTurquoise2, monomeric Midoriishi-Cyan, TagCFP, mTFP1, EGFP, Emerald, Superfolder GFP, Monomeric Azami Green, TagGFP2, mUKG, mWasabi, Clover, mNeonGreen, EYFP, Citrine, Venus, SYFP2, TagYFP, Monomeric Kusabira-Orange, mKOκ, mKO2, mOrange, mOrange2, mRaspberry, mCherry, mStrawberry, mTangerine, tdTomato, TagRFP, TagRFP-T, mApple, mRuby, mRuby2, mPlum, HcRed-Tandem, mKate2, mNeptune, NirFP, TagRFP657, IFP1.4, and iRFP.

17. (canceled)

18. The method of claim 15, wherein the culture fitness function comprises more than one fitness measurement.

19. The method of claim 1, wherein the culture fitness function comprises a turbidity and/or fluorescence measurement.

20. The method of claim 15, wherein the stress ramp function comprises more than one microbial stress.

21. The method of claim 15, wherein the stress ramp function comprises an antibiotic, an antiseptic, a temperature, an aerobic, an anaerobic, an infectious, a nutrient, an irradiative, a pH, a metabolic, and/or a mechanical stress.

22. The method of claim 15, wherein the stress ramp function comprises an increase or decrease in temperature.

23. The method of claim 15, wherein the microbial cell:
is selected from the group consisting of an archaea, a bacterium, a fungi, a protista, a microbial merger or symbiont, and a planarian; or
is selected from the group consisting of a mammalian cell, a plant cell, and an insect cell.

24. (canceled)

25. The method of claim 15, wherein the continuous culture system:
comprises integrated wetware, hardware, and software modules that can be readily interchanged and reconfigured;
comprises a turbidostat with fluorescence detection;
is configured to allow for vial-to-vial culture transfer;
is configured to allow for continuous mixing of the microbial suspensions; or
a combination thereof.

26.-41. (canceled)

42. A continuous culture system configured for high-throughput microbial evolution studies wherein the system configuration comprises at least one stress ramp function that is overlaid on top of at least one culture fitness function, wherein the relationship between the at least one stress ramp function and the at least one fitness function responds to increased culture fitness with increased application of stress in real-time.

43. The continuous culture system of claim 42, wherein the continuous culture system:
comprises integrated wetware, hardware, and software modules that can be readily interchanged and reconfigured;
is configured as a turbidostat with fluorescence detection to measure circuit output and track the loss or gain of circuit function over time;
is configured to allow for vial-to-vial culture transfer;
is configured to allow for continuous mixing of individual cultured species into a community culture;
is configured for long-term continuous culture;
or a combination thereof.

44.-47. (canceled)


SEQUENCE LISTING (44 sequences)

SEQ ID NO: 1 - DNA, 18 bp, Artificial Sequence
  Synthetic polynucleotide
gatgtccacgaggtctct

SEQ ID NO: 2 - DNA, 17 bp, Artificial Sequence
  Synthetic polynucleotide
gtcgacctgcagcgtac

SEQ ID NO: 3 - DNA, 20 bp, Artificial Sequence
  Synthetic polynucleotide
ctgcagcgaggagccgtaat

SEQ ID NO: 4 - DNA, 32 bp, Artificial Sequence
  Synthetic polynucleotide
gaaatcaactacacgtaccataaaatatacag

SEQ ID NO: 5 - DNA, 29 bp, Artificial Sequence
  Synthetic polynucleotide
caactgtcaaccgaatcaaattttaaaag

SEQ ID NO: 6 - DNA, 38 bp, Artificial Sequence
  Synthetic polynucleotide
gtcttattcttaatcgtttatagtagcaacaatatatc

SEQ ID NO: 7 - DNA, 22 bp, Artificial Sequence
  Synthetic polynucleotide
tcgtggactagagcaagatttc

SEQ ID NO: 8 - DNA, 27 bp, Artificial Sequence
  Synthetic polynucleotide
catatcctcataagcagcaatcaattc

SEQ ID NO: 9 - DNA, 51 bp, Artificial Sequence
  Synthetic polynucleotide
acactctttccctacacgacgctcttccgatctgatgtccacgaggtctct

SEQ ID NO: 10 - DNA, 49 bp, Artificial Sequence
  Synthetic polynucleotide
gactggagttcagacgtgtgctcttccgatctgtcgacctgcagcgtac

SEQ ID NO: 11 - DNA, 57 bp, Artificial Sequence
  Synthetic polynucleotide
aatgatacggcgaccaccgagatctacacaactcgctacactctttccctacacgac

SEQ ID NO: 12 - DNA, 57 bp, Artificial Sequence
  Synthetic polynucleotide
aatgatacggcgaccaccgagatctacacttgagcgaacactctttccctacacgac

SEQ ID NO: 13 - DNA, 57 bp, Artificial Sequence
  Synthetic polynucleotide
aatgatacggcgaccaccgagatctacacaagccattacactctttccctacacgac

SEQ ID NO: 14 - DNA, 57 bp, Artificial Sequence
  Synthetic polynucleotide
aatgatacggcgaccaccgagatctacacttcggtaaacactctttccctacacgac

SEQ ID NO: 15 - DNA, 53 bp, Artificial Sequence
  Synthetic polynucleotide
caagcagaagacggcatacgagattaaggcgagtgactggagttcagacgtgt

SEQ ID NO: 16 - DNA, 53 bp, Artificial Sequence
  Synthetic polynucleotide
caagcagaagacggcatacgagatcgtactaggtgactggagttcagacgtgt

SEQ ID NO: 17 - DNA, 53 bp, Artificial Sequence
  Synthetic polynucleotide
caagcagaagacggcatacgagataggcagaagtgactggagttcagacgtgt

SEQ ID NO: 18 - DNA, 53 bp, Artificial Sequence
  Synthetic polynucleotide
caagcagaagacggcatacgagattcctgagcgtgactggagttcagacgtgt

SEQ ID NO: 19 - DNA, 53 bp, Artificial Sequence
  Synthetic polynucleotide
caagcagaagacggcatacgagatggactcctgtgactggagttcagacgtgt

SEQ ID NO: 20 - DNA, 53 bp, Artificial Sequence
  Synthetic polynucleotide
caagcagaagacggcatacgagattaggcatggtgactggagttcagacgtgt

SEQ ID NO: 21 - DNA, 53 bp, Artificial Sequence
  Synthetic polynucleotide
caagcagaagacggcatacgagatctctctacgtgactggagttcagacgtgt

SEQ ID NO: 22 - DNA, 53 bp, Artificial Sequence
  Synthetic polynucleotide
caagcagaagacggcatacgagatcgaggctggtgactggagttcagacgtgt

SEQ ID NO: 23 - DNA, 53 bp, Artificial Sequence
  Synthetic polynucleotide
caagcagaagacggcatacgagataagaggcagtgactggagttcagacgtgt

SEQ ID NO: 24 - DNA, 53 bp, Artificial Sequence
  Synthetic polynucleotide
caagcagaagacggcatacgagatgtagaggagtgactggagttcagacgtgt

SEQ ID NO: 25 - DNA, 53 bp, Artificial Sequence
  Synthetic polynucleotide
caagcagaagacggcatacgagatgctcatgagtgactggagttcagacgtgt

SEQ ID NO: 26 - DNA, 53 bp, Artificial Sequence
  Synthetic polynucleotide
caagcagaagacggcatacgagatatctcagggtgactggagttcagacgtgt

SEQ ID NO: 27 - DNA, 53 bp, Artificial Sequence
  Synthetic polynucleotide
caagcagaagacggcatacgagatactcgctagtgactggagttcagacgtgt

SEQ ID NO: 28 - DNA, 53 bp, Artificial Sequence
  Synthetic polynucleotide
caagcagaagacggcatacgagatggagctacgtgactggagttcagacgtgt

SEQ ID NO: 29 - DNA, 53 bp, Artificial Sequence
  Synthetic polynucleotide
caagcagaagacggcatacgagatgcgtagtagtgactggagttcagacgtgt

SEQ ID NO: 30 - DNA, 53 bp, Artificial Sequence
  Synthetic polynucleotide
caagcagaagacggcatacgagatcggagcctgtgactggagttcagacgtgt

SEQ ID NO: 31 - DNA, 17 bp, Artificial Sequence
  Synthetic polynucleotide
tgaagtggttgcagagg

SEQ ID NO: 32 - DNA, 19 bp, Artificial Sequence
  Synthetic polynucleotide
ccacttgtgatgaggcttg

SEQ ID NO: 33 - DNA, 23 bp, Artificial Sequence
  Synthetic polynucleotide
ggaagctcattatcgagtacttc

SEQ ID NO: 34 - DNA, 19 bp, Artificial Sequence
  Synthetic polynucleotide
cgaatagcgcatattgcac

SEQ ID NO: 35 - DNA, 20 bp, Artificial Sequence
  Synthetic polynucleotide
gactgtacttttctgatgcg

SEQ ID NO: 36 - DNA, 23 bp, Artificial Sequence
  Synthetic polynucleotide
ctacattgggtatcactcaagtc

SEQ ID NO: 37 - DNA, 20 bp, Artificial Sequence
  Synthetic polynucleotide
ctgcgatgctatccatttac

SEQ ID NO: 38 - DNA, 21 bp, Artificial Sequence
  Synthetic polynucleotide
cggtaacagcatcttgcatag

SEQ ID NO: 39 - protein, 23 aa, Artificial Sequence
  Synthetic polypeptide
QETLNSSKPPKKCRRFYGQVPFL

SEQ ID NO: 40 - DNA, 69 bp, Artificial Sequence
  Synthetic polynucleotide
caggagactttgaactcctctaaaccccctaaaaaatgtagaaggttctacgggcaggtgccattcctg

SEQ ID NO: 41 - DNA, 69 bp, Artificial Sequence
  Synthetic polynucleotide
caggagactttgaactcctctaatgccctttaaaaatgtagaaggttctacgggcaggtgccattcctg

SEQ ID NO: 42 - DNA, 69 bp, Artificial Sequence
  Synthetic polynucleotide
caggagactttgaactcctctaatgccgtttaaaaatgtagaaggttctacgggcaggtgccattcctg

SEQ ID NO: 43 - DNA, 69 bp, Artificial Sequence; identical to SEQ ID NO: 41
  Synthetic polynucleotide

SEQ ID NO: 44 - protein, 10 aa, Artificial Sequence
  Synthetic polypeptide
QETLNSSNAL